(19) United States
(12) Patent Application Publication
(10) Pub. No.: US 2023/0330142 A1
(43) Pub. Date: Oct. 19, 2023

(54) COMBINATORIAL ANTIGEN RECOGNITION IN CANCER T CELL THERAPIES

(71) Applicants: The Regents of the University of California, Oakland, CA (US); THE TRUSTEES OF PRINCETON UNIVERSITY, Princeton, NJ (US); THE SIMONS FOUNDATION, INC., New York, NY (US)

(72) Inventors: Ruth DANNENFELSER, Princeton, NJ (US); Gregory ALLEN, San Francisco, CA (US); Olga G. TROYANSKAYA, Princeton, NJ (US); Wendell A. LIM, San Francisco, CA (US)

(21) Appl. No.: 18/017,849

(22) PCT Filed: Aug. 12, 2021
(86) PCT No.: PCT/US2021/045796

§ 371 Date: Jan. 24, 2023

Related U.S. Application Data
(60) Provisional application No. 63/065,726, filed on Aug. 14, 2020.

(51) Int. Cl. A61K 35/17; C07K 14/725; A61P 35/00

(52) U.S. Cl. CPC A61K 35/17; C07K 14/7051; A61P 35/00

(57) ABSTRACT

The present disclosure provides immune cells genetically modified to produce two antigen-triggered polypeptides, each recognizing a different cell surface antigen, wherein the two different cell surface antigens employed are selected from those pairs described herein. The present disclosure further provides systems comprising two antigen-triggered polypeptides (or nucleic acids encoding same), each recognizing a different cell surface antigen, wherein the two different cell surface antigens employed are selected from those pairs described herein. Also provided are method of killing a target cancer cell, using the described genetically modified immune cells and/or systems. The present disclosure also provides polyspecific-immune inducing polypeptides including first and second antigen binding domains specific for first and second antigens, respectively, present on the surface of a target cancer cell.

### CROSS-REFERENCING

This application claims the benefit of U.S. provisional application Ser. No. 63/065,726, filed on Aug. 14, 2020, which application is incorporated by reference herein.

### STATEMENT REGARDING FEDERALLY SPONSORED RESEARCH

This invention was made with government support under grants P50 GM081879, R01 CA196277 and R01 GM071966 awarded by The National Institutes of Health. The government has certain rights in the invention.

### INTRODUCTION

Despite recent clinical success in using engineered T cells to treat hematologic cancers (Maude et al., 2018; Neelapu et al., 2017), a major barrier in expanding their use to solid tumors is the challenge of specific tumor recognition. While it is possible to engineer chimeric antigen receptors (CARs) directed toward tumor associated antigens, many of those antigens, especially in the case of solid tumors, are also expressed, often at lower levels, in other normal tissues, leading to cases of toxic cross-reactivity (Lamers et al., 2013; Morgan et al., 2010; Parkhurst et al., 2011; Thistlethwaite et al., 2017). While toxicity can in some cases be ameliorated by reducing CAR T dosage, the small therapeutic window caused by poor discrimination leads to a tradeoff between efficacy and toxicity. The difficulty of finding absolutely tumor unique surface antigens that can be distinctly recognized by CARs has led some to question the capability of such engineered T cells to ultimately achieve success in safely treating solid tumors (Rosenberg and Restifo, 2015).

Current approaches for engineering CAR T cells, however, focus only on recognition of a single target antigen. If one considers that solid tumors express an array of antigens, it is possible that improved specificity could be achieved through recognition of combinatorial antigen signatures. Such considerations, however, have only recently become actionable with advances in synthetic biology approaches to engineering T cell therapies. Engineered cells are unique among therapeutic modalities in that they can in principle be engineered with multi-antigen recognition circuits. For example, recent advances have shown that it is possible to engineer CAR T cells that recognize target cells with combinatorial Boolean logic: one can engineer T cells with multi-receptor circuits that function as AND gates (requiring two antigens to be present) (Kloss et al., 2013; Roybal et al., 2016a, 2016b; Srivastava et al., 2019; Wilkie et al., 2012), NOT gates (Fedorov et al., 2013), and OR gates (requiring the presence of one of two possible antigens) (Grada et al., 2013; Hegde et al., 2013). AND gates (high expression of two antigens) and NOT gates (high expression of one antigen, low expression of another) should significantly increase tumor selectivity by limiting cross-reactivity with healthy tissues that also express the CAR/TCR target antigen. Engineering T cells with more complex recognition circuits based on more than two antigens is possible.

This disclosure provides several multigene-antigen recognition circuits that rely on Boolean logic.

### SUMMARY

The present disclosure provides immune cells genetically modified to produce two or more antigen-triggered polypeptides, each recognizing a different cell surface antigen, wherein the two or more different different cell surface antigens employed are selected from those pairs and triplets described herein. The present disclosure further provides systems comprising two or three antigen-triggered polypeptides (or nucleic acids encoding same), each recognizing a different cell surface antigen, wherein the two different cell surface antigens employed are selected from those pairs described herein. Also provided are method of killing a target cancer cell, using the described genetically modified immune cells and/or systems. The present disclosure also provides polyspecific-immune inducing polypeptides including first and second antigen binding domains specific for first and second antigens, respectively, present on the surface of a target cancer cell.

Precise discrimination of tumor from normal tissues remains a major roadblock for therapeutic efficacy of chimeric antigen receptors (CAR) T cells. Here, a comprehensive in silico screen is performed to identify multi-antigen signatures that improve tumor discrimination by CAR T cells engineered to integrate multiple antigen inputs via Boolean logic, e.g. AND and NOT. >2.5 million dual antigens and ˜60 million triple antigens were screened across 33 tumor types and 34 normal tissues. It was found that dual antigens significantly outperform the best single clinically investigated CAR targets and confirm key predictions experimentally. Further, antigen triplets were identified which are predicted to show close to ideal tumor-versus-normal tissue discrimination for several tumor types. This work demonstrates the potential of 2-3 antigen Boolean logic gates for improving tumor discrimination by CAR T cell therapies.

In the study presented below, a comprehensive computational search of all possible pairs of predicted surface antigens in the human genome (2,358 total predicted surface genes with >2.5 million total possible surface-presented antigen combinations) were analyzed to explore the strategy of tumor cell targeting by CAR T cells engineered to express multi-receptor circuits that function as Boolean logic gates. All AND and NOT gates were scored by how well the putative combination separates tumor and normal tissue samples for 33 distinct tumor types and 34 major healthy tissues, and then add a third surface antigen to explore more than 60 million additional unique AND and NOT gates for triplets. For these logic gates, both how much off-target toxicity can be avoided (precision) and the potential number of tumor samples that can be targeted (recall) were defined.

It was found find that cellular recognition programs which incorporate information from multiple (2 or 3) antigens, outperform standard single antigen recognition circuits. As the number of antigens used to discriminate tumor vs normal tissue is increased, the precision of tumor detection increases at the cost of decreased recall of all tumor specimens. For most cancer types, there are numerous dual antigen combinations that significantly improve the precision and recall of the best single antigen, including currently clinically investigated CAR targets. For several tumor types, antigen triplets are predicted to show close to ideal tumor-versus-normal tissue discrimination. Improved detection of Renal Cell Carcinoma was experimentally validated using computationally identified antigen pairs for proof-of-principle. In total, the study illustrates an overall strategy for merging computational analysis with increasing synthetic biology capabilities to identify and target sectors of antigen recognition space that precisely identify and discriminate particular tumor types.

Antigen doublet and triplet circuits, their Boolean relationships and the cancers that can be targeted using immune cells that have been programmed to recognize those antigens are listed in Table 1 below.

In some embodiments, an in vitro or ex vivo genetically modified cytotoxic immune cell is provided. In these embodiments, the cytotoxic immune cell may be genetically modified to produce at least two different polypeptides and wherein the polypeptides bind to a selected antigen doublet or triplet of Table 1. For example, selecting the first antigen doublet from Table 1, the cell should produces a first polypeptide that binds to SPN and a second polypeptide that binds to ERBB2; selecting the first antigen triplet from Table 1, the cell should produces a first polypeptide that binds to GPR143, a second polypeptide that binds to MLANA and a third polypeptide that binds to ROR1.

In some embodiments, the different polypeptides are independently selected from the group consisting of a binding-triggered transcriptional switch (BTTS), a chimeric antigen receptor (CAR), a T cell receptor (TCR), and an inhibitory CAR, depending on the Boolean operators associated with the selected combination of cell surface antigens. For example, the Boolean conditions of the first antigen doublet listed in Table 1 (“SPN AND NOT-ERBB2”) can be satisfied by a cell that produces a CAR that binds to SPN and an iCAR that binds to ERBB2 (although there are many other arrangements described below). In this example, the immune cell is not activated if the polypeptides bind to SPN AND ERBB2. Rather, the cell is only activated if the cell binds to SPEN and not ERBB2. Likewise, the Boolean conditions of the first antigen triplet listed in Table 1 (“GPR143 AND MLANA AND NOT-ROR1”) can be satisfied by a cell that produces (a) a BTTS that binds to GPR143, (b) CAR that binds to MLANA, where activation of the BTTS by binding to GPR143 induces expression of the CAR and (c) an iCAR that bind to ROR1 (although there are many other arrangements described below). In this example, the immune cell is not activated if the polypeptides bind to GPR143, MLANA AND ROR1. Rather, the cell is only activated if the cell binds to GPR143 and MLANA and not ROR1.

In some embodiments, the immune cell is genetically modified to produce two different polypeptides comprising a first polypeptide and a second polypeptide, wherein the first polypeptide specifically binds to a first antigen of a selected antigen doublet from Table 1 and the second polypeptide specifically binds to a second antigen of the selected antigen doublet. In some cases, the immune cell is only activated if the first and second polypeptides are both bound to an antigen whereas in others the immune cell is not activated if the first and second peptides are both bound to an antigen.

In some embodiments, the immune cell is genetically modified to produce three different polypeptides comprising a first polypeptide, a second polypeptide and a third polypeptide, wherein the first polypeptide specifically binds to a first antigen of a selected antigen doublet from Table 1, the second polypeptide specifically binds to a second antigen of the selected antigen doublet and the third polypeptide specifically binds to a third antigen of the selected antigen doublet. In some cases this immune cell is only activated if the first, second and third polypeptides are bound to an antigen whereas in other cases the immune cell is not activated if the first, second and third peptides are bound to an antigen.

The polypeptides may each comprise an extracellular binding domain independently selected from an antibody, peptide or ligand for a receptor.

In any embodiment, the cell can be a cytotoxic T cell, although other types of immune cells may be used under certain circumstances.

Also provided is method of killing a target cancer cell in an individual. In these embodiments the method may comprise: administering to the individual an effective number of the genetically modified cytotoxic immune cell, as summarized above, wherein said genetically modified cytotoxic immune cell kills the target cancer cell in the individual, and the type of cancer cell is associated with selected antigen doublet or triplet of Table 1. For example, for a cell that produces polypeptides that recognize the first antigen doublet listed in Table 1 (“SPN AND NOT-ERBB2”) the cancer cell can be an acute myeloid leukemia cell. Likewise, for a cell that produces a polypeptide that recognize the first antigen triplet listed in Table 1 (“GPR143 AND MLANA AND NOT-ROR1”) the cancer cell can be a uveal melanoma cell.

Also provided is a system for killing a target cancer cell. In these embodiments the system may comprise: a) a first antigen-triggered polypeptide that binds specifically to a first target antigen or a nucleic acid encoding the same; and b) a second antigen-triggered polypeptide that binds specifically to a second target antigen or a nucleic acid encoding the same; and, optionally c) a third antigen-triggered polypeptide that binds specifically to a third target antigen or a nucleic acid encoding the same. In these embodiments, the first, second and optional third antigen-triggered polypeptides bind to a selected antigen doublet or triplet of Table 1. Consistent with the above, the first, second and optional third polypeptides are independently selected from the group consisting of a binding-triggered transcriptional switch (BTTS), a chimeric antigen receptor (CAR), a T cell receptor (TCR), and an inhibitory CAR, depending on the Boolean operators associated with the selected combination of cell surface antigens. As would apparent, this method may be done by administering a cell to an individual.

Also provided is a method of killing a target cancer cell in an individual. In these embodiments the method may comprise: a) introducing the system summarized into a cytotoxic T cell in vitro or ex vivo, generating a modified cytotoxic T cell; and b) administering the modified cytotoxic T cell to the individual. In these embodiments, the target cancel cell is associated with selected antigen doublet or triplet of Table 1.

Also provided is polyspecific-immune-inducing polypeptide (PIIP) comprising: a first antigen binding domain specific for a first antigen present on the surface of a target cancer cell, a second antigen binding domain specific for a second antigen present on the surface of the target cancer cell, and, optionally, a third antigen binding domain specific for a third antigen present on the surface of the target cancer cell, wherein the polyspecific-immune-inducing polypeptide binds a selected antigen doublet or triplet of Table 1. In some embodiments, the polyspecific-immune-inducing polypeptide is a polyspecific antibody (e.g., a bi-specific or tri-specific antibody). In other embodiments, the polyspecific-immune-inducing polypeptide may be a polyspecific chimeric antigen receptor (CAR) or polyspecific T cell receptor (TCR).

Also provided is an in vitro or ex vivo genetically modified cytotoxic immune cell, wherein the cytotoxic immune cell is genetically modified to produce a polyspecific-immune-inducing polypeptide as summarized above.

Also provided is a method of killing a target cancer cell in an individual, the method comprising administering to the individual an effective amount of a polyspecific-immune-inducing polypeptide as summarized above. This method may involve administering to the individual an effective amount of cytotoxic immune cells genetically modified to produce the PIIP.

### BRIEF DESCRIPTION OF THE DRAWINGS

FIG. 1. Computationally enumerating combinatorial antigen sets predicted to improve T cell discrimination of cancer vs normal cells. A) Single antigen targets for CAR T cells often show cross reactivity with subset of normal tissues. Combinatorial recognition circuits (AND, NOT, etc) could improve discrimination. B) Single antigen targets theoretically hit samples that have high expression of antigen A or B. Using boolean T cells we can target specific patterns of antigen expression reducing off target toxicity. C) Computational pipeline for identifying antigen pairs with improved tumor discrimination. For each cancer type (N=33), normalized RNAseq expression data is combined with RNAseq data for 34 normal tissues. All potential transmembrane antigen pairs are then evaluated for their potential to separate samples of a given tumor type from all normal samples in expression space. Shaded boxes highlight specific steps of the pipeline starting with a representation of the expression data, followed by the scoring method, and toy examples highlighting how evaluation metrics are calculated.

FIG. 2. Dual antigen use greatly improves the precision of cancer detection. Antigen combinations were ranked by their clustering scores for each tumor and each gate type (e.g., clinical, novel, clinical-clinical, clinical-novel, or novel-novel). In this figure different subsets of the top antigens (e.g., the top scoring singlet/pair or the top 10 combinations) are taken and their F₁ scores are used to describe their potential discriminatory power. A) Distribution of tumor vs normal discrimination scores (F₁) for the top clinical antigens or top 10 novel antigens for each cancer type, and for the top 10 antigen pairs (clinical-clinical, clinical-novel, or novel-novel) for each cancer type. F₁ scores range between 0 (no sensitivity and specificity) and 1 (perfect precision and recall). Here we see significant gains in discrimination power going from a clinical antigen to a single novel antigen (p=8.41×10⁻⁶⁹; n=646) and from a clinical-clinical antigen pair to a clinical-novel pair (p=1.38×10⁻¹¹; n=660). B) Improvement in tumor vs normal discrimination with dual antigen recognition by cancer type. F₁ scores are shown for the highest clustering score single clinical antigen and the highest clustering score dual antigen pair. All antigen pairs improve over the highest performing single clinical antigen. C) Pie chart showing the composition of different gate types of pairs in the top 10 per tumor type. A AND B gates have high expression of both antigens, A AND NOT B have high expression of one antigen and low expression of the second antigen. The majority of pairs are AND NOT gates. D) Novel antigens (hubs, blue) identified that form high performing pairs with numerous current clinically targeted CAR antigens (spokes, orange). Edge weights and color correspond to the number of applicable cancer types.

FIG. 3. Numerous potential antigen pairs show significant improvement in the precision of tumor recognition. A) Examples of antigen pairs with improved tumor vs normal discrimination by switching from single to dual antigen recognition. 2D plots show expression level of both antigens in normal tissue samples (grey) vs specific cancer type samples (red). Navy circles show centroids for each of the normal tissue types (labeled when close to red cancer cluster). Pairs were scored by clustering as well as by F₁ score. Density function of single antigen expression in tumor (red) and normal (grey) tissue are plotted on respective axis, including an optimal point of discrimination showing the best potential tumor vs normal discrimination using a single antigen. B) Example 2D plots as in (A) highlighting potential AND gates that combine known CAR target pairs (clinical-clinical), known CAR targets paired with new potential antigens (clinical-novel) and pairs of new potential targets (novel-novel). C) Example 2D plots as in (B) highlighting potential NOT gates.

FIG. 4. Computationally predicted antigen pairs can be constructed as AND-gated CAR T cells in a laboratory setting, with precise in-vitro discrimination A) RCC recognition circuit: CD70 and AXL. Segregation of RCC samples (red points) vs normal tissue samples (grey points) in antigen expression space, highlighting overlap of CD70 expression with normal blood samples (green points). We constructed an anti-AXL synNotch receptor and validated that human T cells expressing the receptor can detect 769-P Renal Cell Cancer cell line (CD70+AXL+), but not Raji B-cell line (CD70+AXL−) via FAC detection of GFP reporter induction. In cell killing assays, we compared human primary CD8⁺ T cells constitutively expressing the anti-CD70 CAR with the same cells transfected with the anti-AXL synNotch 4 anti-CD70 CAR AND-gate circuit. The single antigen targeting anti-CD70 CAR T cells killed both RCC and B-cell lines, while the circuit T cells selectively killed RCC cells (n=3, p value from unpaired two sample student's T-test). B) RCC recognition circuit: AXL and CDH6. Segregation of RCC samples (red points) vs normal tissue samples (grey points) in antigen expression space, highlighting overlap of AXL expression with normal lung samples (green points). We constructed an anti-CDH6 synNotch receptor and validated that human T cells expressing the receptor can detect 769-P Renal Cell Cancer cell line (AXL+CDH6+), but not the Beas2B lung epithelial cell line (AXL+CDH6−) via FAC detection of GFP reporter induction. In cell killing assays, we compared human primary CD8⁺ T cells constitutively expressing the anti-AXL CAR with the same cells transfected with the anti-CDH6 synNotch→anti-AXL CAR AND-gate circuit. The single antigen targeting anti-AXL CAR T cells killed both RCC and lung cell lines, while the circuit T cells selectively killed RCC cells (n=3, p value from unpaired two sample student's T-test).

FIG. 5. Antigen triplets can significantly improve recognition of challenging cancers with some reduction in sensitivity A) (left) Distribution of tumor vs normal discrimination scores (F₁) for top 10 antigen singlets, doublets, and triplets. We see significant performance improvements going from 1 to 2 antigens (p=7.68×10⁻⁶⁸; n=2979) and 2 to 3 antigens (p=2.83×10⁻⁴⁸; n=1578). The same plot is shown on the right for top 10 clinical antigen singlets, clinical-novel antigen doublets, and clinical-clinical-novel antigen triplets. Again, we see significant increases in performance going from clinical to clinical-novel pairs (p=6.06×10⁻⁸¹; n=676) and from clinical-novel pairs to clinical-clinical-novel triplets (p=5.00×10⁻⁸; n=438). F₁ scores range between 0 (no sensitivity and specificity) and 1 (perfect precision and recall). B) Improvement in tumor vs normal discrimination with triplet antigen recognition by cancer type. F₁ score ranges from best single clinical antigen (grey circle) to best double with at least one clinical antigen (blue circle) to best triplet with a least one clinical antigen. C) Pie chart showing the composition of different gate types (high:high:high, high:high:low, high:low:low) of triplets in the top 100 per tumor type. D) Each grey dot represents the precision (left) or recall (right) for one of the top antigens (single, double, and triples) for a single tumor type. Grey lines show the median illustrating the global increase in precision when including more antigens at the expense of recall. Precision has a significant increase and recall a significant decrease when going from one to two (precision: Wilcoxon rank sum p=2.45×10⁻¹²⁰, n=2979; recall: Wilcoxon rank sum p=3.55×10⁻⁹, n=2979) and two to three antigens (precision: Wilcoxon rank sum p=5.69×10⁻⁸⁴, n=1578; recall: Wilcoxon rank sum p=5.94×10⁻⁴; n=1578). E) Example 3D triplet antigen gates showing expression level of all antigens in normal tissue samples (grey) vs specific cancer type samples (red). Tissue centroids are in dark blue. Triplets were scored by clustering as well as by F₁ score.

FIG. 6. In silico T cell circuit design. In silico analysis of tumor vs normal expression data can be used to identify discriminatory antigen patterns. These potential antigen signatures can then be used as the basis for synthetic biology engineering of precision therapeutic T cells.

FIGS. 7A-7B provide schematic depictions of various antigen-triggered polypeptides; and AND and AND-NOT logic gates using the antigen-triggered polypeptides.

FIGS. 8A-8G provide schematic depictions of exemplary synNotch receptor Notch regulatory regions.

FIGS. 9A-9F provide schematic depictions of exemplary split CARs.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

“T cell” includes all types of immune cells expressing CD3 including T-helper cells (CD4+ cells), cytotoxic T-cells (CD8+ cells), T-regulatory cells (Treg) and gamma-delta T cells.

A “cytotoxic cell” includes CD8⁺ T cells, natural-killer (NK) cells, and neutrophils, which cells are capable of mediating cytotoxicity responses.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc. In some cases, the individual is a human.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

By “specifically binds” or “selectively bind” is meant that the molecule binds preferentially to the target of interest or binds with greater affinity to the target than to other molecules. For example, a DNA molecule will bind to a substantially complementary sequence and not to unrelated sequences. Specific binding may refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “antibody” and “immunoglobulin”, as used herein, are used interchangeably may generally refer to whole or intact molecules or fragments thereof and modified and/or conjugated antibodies or fragments thereof that have been modified and/or conjugated. The immunoglobulins can be divided into five different classes, based on differences in the amino acid sequences in the constant region of the heavy chains. All immunoglobulins within a given class will have very similar heavy chain constant regions. These differences can be detected by sequence studies or more commonly by serological means (i.e. by the use of antibodies directed to these differences). Immunoglobulin classes include IgG (Gamma heavy chains), IgM (Mu heavy chains), IgA (Alpha heavy chains), IgD (Delta heavy chains), and IgE (Epsilon heavy chains).

Antibody or immunoglobulin may refer to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized, see for instance Fundamental Immunology Ch. 7 (Paul, W., ed., 2nd ed. Raven Press, N.Y. (1989)). Briefly, each heavy chain typically is comprised of a heavy chain variable region (abbreviated as V_{H}) and a heavy chain constant region (abbreviated as C_{H}). The heavy chain constant region typically is comprised of three domains, C_{H}1, C_{H}2, and C_{H}3. Each light chain typically is comprised of a light chain variable region (abbreviated as V_{L}) and a light chain constant region (abbreviated herein as C_{L}). The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Whole or largely intact antibodies are generally multivalent, meaning they may simultaneously bind more than one molecule of antigen whereas antibody fragments may be monovalent. Antibodies produced by an organism as part of an immune response are generally monospecific, meaning they generally bind a single species of antigen. Multivalent monospecific antibodies, i.e. antibodies that bind more than one molecule of a single species of antigen, may bind a single antigen epitope (e.g., a monoclonal antibody) or multiple different antigen epitopes (e.g., a polyclonal antibody).

Multispecific (e.g., bispecific) antibodies, which bind multiple species of antigen, may be readily engineered by those of ordinary skill in the art and, thus, may be encompassed within the use of the term “antibody” used herein where appropriate. Also, multivalent antibody fragments may be engineered, e.g., by the linking of two monovalent antibody fragments. As such, bivalent and/or multivalent antibody fragments may be encompassed within the use of the term “antibody”, where appropriate, as the ordinary skilled artisan will be readily aware of antibody fragments, e.g., those described below, which may be linked in any convenient and appropriate combination to generate multivalent monospecific or polyspecific (e.g., bispecific) antibody fragments.

Antibody fragments include but are not limited to antigen-binding fragments (Fab or F(ab), including Fab′ or F(ab′), (Fab)₂, F(ab′)₂, etc.), single chain variable fragments (scFv or Fv), “third generation” (3G) molecules, etc. which are capable of binding the epitopic determinant. These antibody fragments retain some ability to selectively bind to the subject antigen, examples of which include, but are not limited to:
(1) Fab, the fragment which contains a monovalent antigen-binding fragment of an antibody molecule can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab)₂, the fragment of the antibody that can be obtained by treating whole antibody with the enzyme pepsin without subsequent reduction;
(4) F(ab)₂ is a dimer of two Fab′ fragments held together by two disulfide bonds;
(5) Fv, defined as a genetically engineered fragment containing the variable region of the light chain and the variable region of the heavy chain expressed as two chains;
(6) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, tetrabodies, etc. which may or may not be polyspecific (see, for example, WO 94/07921 and WO 98/44001) and
(7) “3G”, including single domain (typically a variable heavy domain devoid of a light chain) and “miniaturized” antibody molecules (typically a full-sized Ab or mAb in which non-essential domains have been removed).

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides or polypeptides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a target antigen” includes a plurality of such antigens and reference to “the system” includes reference to one or more systems and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

Some of the following description refers to logic circuits that use antigen pairs and/or polypeptides that bind to the same. The same concepts can be readily extended to antigen triplets. For example, if an antigen is listed as being a “NOT” antigen, activation of the cell should be inhibited when the cell binds to the antigen, regardless of whether the “NOT” antigen is part of an antigen pair or triplet.

The present disclosure provides an immune cell genetically modified to produce at least two (i.e., two or three or more) antigen-triggered polypeptides, each recognizing a different cell surface antigen, wherein the at least two different cell surface antigens employed are selected from the pairs and triplets described in Table 1. The present disclosure provides a system comprising two or three antigen-triggered polypeptides, each recognizing a different cell surface antigen, wherein the two or three different cell surface antigens employed are selected from those pairs described herein. The present disclosure provides a method of killing a target cancer cell, using a genetically modified immune cell or a system of the present disclosure. Also provided are polyspecific-immune-inducing polypeptides (PIIP) having a first antigen binding domain specific for a first antigen present on the surface of a target cancer cell, a second antigen binding domain specific for a second antigen present on the surface of the target cancer cell and, optionally, a third antigen binding domain specific for a third antigen present on the surface of the target cancer cell.

The present disclosure provides an in vitro or ex vivo genetically modified cytotoxic immune cell, where the cytotoxic immune cell is genetically modified to produce two or three different antigen-triggered polypeptides that recognize a corresponding number of different cell surface antigens, and where at least one of the two or three different cell surface antigens is present on the surface of a target cancer cell. In some cases, the different antigen-triggered polypeptides may comprise: a) a first antigen-triggered polypeptide that binds specifically to a first target cell surface antigen present on a target cancer cell; b) a second antigen-triggered polypeptide that binds specifically to a second target cell surface antigen and, optionally (i.e., in the case of triplets) a third antigen-triggered polypeptide that binds specifically to a third target cell surface antigen. In some cases, the genetically modified cytotoxic immune cell is a genetically modified cytotoxic T cell or a genetically modified natural killer cell. In some cases, the different antigen-triggered polypeptides provide an AND gate; thus, for example, in some cases, the genetically modified cytotoxic immune cell is activated to kill a target cancer cell only when the target cancer cell expresses both of the two or all three different cell surface antigens on its cell surface. In some cases, the different antigen-triggered polypeptides provide an AND-NOT gate; thus, for example, in some cases, the genetically modified cytotoxic immune cell: a) is activated to kill a target cancer cell that expresses the first target cell surface antigen, but not the second and/or third target cell surface antigen, on its cell surface; and b) is inhibited from killing a non-cancerous cell if the non-cancerous cell expresses both the first target cell surface antigen and the second and/or third target cell surface antigen on its cell surface.

The present disclosure provides a system for killing a target cancer cell, the system comprising: a) a first antigen-triggered polypeptide that binds specifically to a first target antigen present on the target cancer cell, or a first nucleic acid comprising a nucleotide sequence encoding the first antigen-triggered polypeptide; b) a second antigen-triggered polypeptide that binds specifically to a second target antigen, or a second nucleic acid comprising a nucleotide sequence encoding the second antigen-triggered polypeptide and, optionally (in the case of antigen triplets) c) a third antigen-triggered polypeptide that binds specifically to a third target antigen, or a third nucleic acid comprising a nucleotide sequence encoding the third antigen-triggered polypeptide. In some cases, the system provides an AND gate; thus, for example, in some cases, the first target antigen and the second and/or third target antigen are all present on the surface of a target cancer cell. In some cases, the system provides an AND-NOT gate; thus, for example, in some cases: a) the first target antigen and the second target antigen are both present on the surface of a non-cancerous cell; and b) the first target antigen, but not the second target antigen, is present on the surface of a target cancer cell. A system of the present disclosure can be introduced ex vivo into an immune cell obtained from a patient, to generate a modified immune cell; and the modified immune cell can be introduced into the patient from whom the immune cell was obtained.

The present disclosure provides a method of killing a target cancer cell in an individual. In some cases, a method of the present disclosure for killing a target cell in an individual comprises: a) introducing a system of the present disclosure into an immune cell (e.g., a CD8⁺ T cell; an NK cell) obtained from the individual, generating a modified immune cell; and b) administering the modified immune cell to the individual, where the modified immune cell kills the target cancer cell in the individual.

The present disclosure provides a method of killing a target cancer cell in an individual. In some cases, a method of the present disclosure for killing a target cell in an individual comprises administering a genetically modified cytotoxic immune cell (e.g., a genetically modified CD8⁺ T cell; a genetically modified NK cell) of the present disclosure to the individual, where the genetically modified immune cell kills the target cancer cell in the individual.

As noted above, a genetically modified cytotoxic immune cell of the present disclosure, and a system of the present disclosure, involve at least two (e.g., two or three) antigen-triggered polypeptides that recognize a corresponding number of different cell surface antigens. A pair or triplet of antigen-triggered polypeptides recognizes and binds to a corresponding number of target antigens; antigen binding activates the antigen-triggered polypeptides. Thus, a first antigen-triggered polypeptide binds a first member of a target antigen pair; a second antigen-triggered polypeptide binds a second member of the target antigen pair, and so on. Target antigen combinations (also referred to herein as “antigen pairs” or “antigen triplets”) are provided in Table 1. At least one of the antigens of a target antigen pair or triplet listed in Table 1 is present on the surface of a target cancer cell. In some cases, a second target antigen of a target antigen pair or triple is present on the surface of the same target cancer cell as the first target antigen of the target antigen pair. In some cases, the first target antigen of the target antigen pair is present on the surface of a target cancer cell, and the second and/or third target antigen of a target antigen pair/triplet is not present on the surface of the same target cancer cell; in these cases, both antigens of the target antigen pair are present on the surface of a non-cancerous cell. The target antigen combinations presented in Table 1 provide for an AND logic gate or an AND-NOT logic gate for a particular cancer cell type. Such logic being represented in Table 1 where an “AND” precedes or follows a target antigen present on the surface of a target cancer cell and a “NOT” precedes an antigen that that is not present on the surface of a target cancer cell, but may be present on the surface of a non-cancerous cell.

Where a target antigen pair/triple provides for an AND logic gate, two or all three antigens must be present on the surface of a target cancer cell in order for a genetically modified cytotoxic immune cell of the present disclosure to kill the target cancer cell, where in this case the genetically modified cytotoxic immune cell is genetically modified to express two or three antigen-triggered polypeptides, each recognizing one of the target antigens of the target antigen pair/triplet. For example, where a target antigen pair present of Table 1 is indicated as providing solely AND gate logic, all three target antigens of the target antigen pair must be present on the surface of a target cancer cell in order for a genetically modified cytotoxic immune cell of the present disclosure to kill the target cancer cell.

Where a target antigen pair/triple provides an AND-NOT logic gate (or, correspondingly, a NOT-AND logic gate), a genetically modified cytotoxic immune cell of the present disclosure: a) is activated to kill a target cancer cell that expresses the AND target cell surface antigen (e.g., the first target cell surface antigen), but not the NOT target cell surface antigen (e.g., the second and/or third target cell surface antigen), on its cell surface; and b) is inhibited from killing a non-cancerous cell if the non-cancerous cell expresses both the AND target cell surface antigen and the NOT target cell surface antigen(s) on its cell surface. In these cases, the genetically modified cytotoxic immune cell must express at least a first antigen-triggered polypeptide that specifically binds the AND target antigen of the target antigen pair and a second triggered polypeptide that specifically binds the NOT antigen of the target antigen pair. For example, in some cases, binding of an antigen-triggered polypeptide to the NOT target cell surface antigen (expressed on a non-cancerous cell) inhibits T cell activation. In this manner, unintended/undesired killing of a non-cancerous cell is reduced, because the target cancer cell expressing the AND target antigen and not the NOT target antigen will be preferentially killed over the non-cancerous cell expressing both the AND target antigen and the NOT target antigen. Since the cancer cell does not express the NOT target cell surface antigen (expressed on a non-cancerous cell), binding of the first antigen-triggered polypeptide to the AND target antigen (present on the cancer cell surface) results in activation of the genetically modified cytotoxic T cell and killing of the cancer cell.

In some cases, the first antigen-triggered polypeptide is a binding triggered transcriptional switch (BTTS) and the second antigen-triggered polypeptide is a chimeric antigen receptor (CAR). In some cases, the first antigen-triggered polypeptide is a BTTS and the second antigen-triggered polypeptide is a T cell receptor (TCR). In some cases, the first antigen-triggered polypeptide is a BTTS, and the second antigen-triggered polypeptide is a split CAR (e.g., an ON-switch CAR). In some cases, the first antigen-triggered polypeptide is a BTTS, and the second antigen-triggered polypeptide is one polypeptide chain of a split CAR (e.g., an ON-switch CAR). In some cases, the first antigen-triggered polypeptide is a BTTS, and the second antigen-triggered polypeptide is another BTTS. Any or either of the first and second antigen-triggered polypeptides of the subject systems may independently be a BTTS, a CAR, a TCR or the like. In some cases, both the first and second antigen-triggered polypeptides of the subject systems may be a BTTS, a CAR, a TCR or the like.

In some cases, the first antigen-triggered polypeptide is a CAR, and the second antigen-triggered polypeptide is an antigen-binding inhibitory polypeptide, such as e.g. an inhibitory CAR (iCAR). In some cases, the first antigen-triggered polypeptide is a TCR, and the second antigen-triggered polypeptide is an antigen-binding inhibitory polypeptide, such as e.g. an iCAR. In some cases, the first antigen-triggered polypeptide is an ON-switch CAR, and the second antigen-triggered polypeptide is an antigen-binding inhibitory polypeptide, such as e.g. an iCAR. In some cases, the first antigen-triggered polypeptide is a CAR, and the second antigen-triggered polypeptide is a BTTS. In some cases, the first antigen-triggered polypeptide is a TCR, and the second antigen-triggered polypeptide is a BTTS. In some cases, the first antigen-triggered polypeptide is an ON-switch CAR, and the second antigen-triggered polypeptide is a BTTS.

In some cases, the target cancer cell is a liposarcoma, a glioblastoma, a breast cancer cell, a renal cancer cell, a pancreatic cancer cell, a melanoma, an anaplastic lymphoma, a leiomyosarcoma, an astrocytoma, an ovarian cancer cell, a neuroblastoma, a mantle cell lymphoma, a sarcoma, a non-small cell lung cancer cell, an AML cell, a stomach cancer cell, a B-cell cancer cell, a lung cancer cell, or an oligodendroglioma.

The description that follows below primary describes antigen pairs. Strategies for antigen triplets can be readily derived from the following description.

### AND Gate Target Antigen Pairs

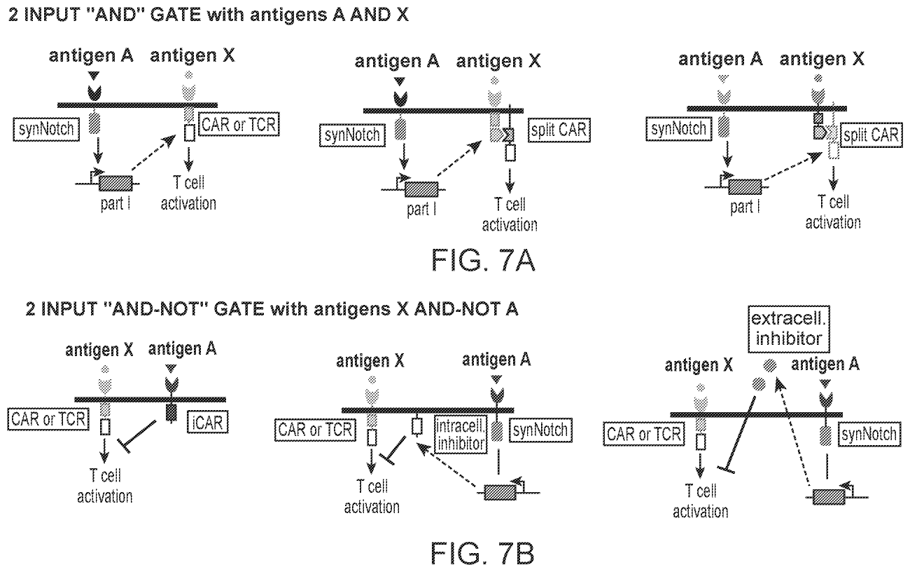

As noted above, in some cases, expression of the second antigen-triggered polypeptide in a genetically modified immune cell is induced only when the first antigen-triggered polypeptide binds to the first target antigen of the target antigen pair, where the binding to the first target antigen activates the first antigen-triggered polypeptide. Non-limiting examples of 2-input AND gates (AND gates based on 2 target antigens) are depicted schematically in FIG. 7.

For example, in some cases, the first antigen-triggered polypeptide is a BTTS and activation of the BTTS by binding to the first antigen (present on a target cancer cell) induces expression of the second antigen-triggered polypeptide. The second antigen-triggered polypeptide binds to the second antigen of the target antigen pair, where the second antigen is expressed on the surface of the target cancer cell. As an example, in some cases, the first antigen-triggered polypeptide is a BTTS and the second antigen-triggered polypeptide is a single chain CAR. As another example, in some cases, the first antigen-triggered polypeptide is a BTTS and the second antigen-triggered polypeptide is a TCR. For example, in some cases, the BTTS comprises an intracellular domain comprising a transcriptional activator, and activation of the BTTS by binding to the first antigen (present on a target cancer cell) induces release of the transcriptional activator; the released transcriptional activator activates transcription of the TCR or the single-chain CAR.

As another example, in some cases, the first antigen-triggered polypeptide is a BTTS and activation of the BTTS by binding to the first antigen (present on a target cancer cell) induces expression of the second antigen-triggered polypeptide, where the second antigen-triggered polypeptide is a heterodimeric (“two chain” or “split”) CAR comprising a first polypeptide chain and a second polypeptide chain. The heterodimeric CAR binds to the second antigen of the target antigen pair, where the second antigen is expressed on the surface of the target cancer cell. For example, in some cases, the first antigen-triggered polypeptide is a BTTS and the second antigen-triggered polypeptide is a split CAR (e.g., an ON-switch CAR). In some cases, activation of the BTTS by binding to the first antigen (present on a target cancer cell) induces expression of only the first polypeptide chain of the heterodimeric CAR; expression of the second polypeptide chain of the heterodimeric CAR can be constitutive. For example, in some cases, the BTTS comprises an intracellular domain comprising a transcriptional activator, and activation of the BTTS by binding to the first antigen (present on a target cancer cell) induces release of the transcriptional activator; the released transcriptional activator activates transcription of the first polypeptide chain of the heterodimeric CAR. In some cases, activation of the BTTS by binding to the first antigen (present on a target cancer cell) induces expression of only the second polypeptide chain of the heterodimeric CAR; expression of the first polypeptide chain of the heterodimeric CAR can be constitutive. Once the first polypeptide chain of the heterodimeric CAR is produced in the cell, it heterodimerizes with the second polypeptide chain of the heterodimeric CAR. As another example, in some cases, the BTTS comprises an intracellular domain comprising a transcriptional activator, and activation of the BTTS by binding to the first antigen (present on a target cancer cell) induces release of the transcriptional activator; the released transcriptional activator activates transcription of the second polypeptide chain of the heterodimeric CAR.

In AND gate systems, unintended/undesired killing of non-target cells is reduced; for example, a cell that expresses on its cell surface only one of the target antigen pair is not killed by a genetically modified cytotoxic immune cell of the present disclosure.

### AND-NOT Gate Target Antigen Pairs

As noted above, in some cases, where a target antigen pair provides an AND-NOT logic gate, a genetically modified cytotoxic immune cell of the present disclosure: a) is activated to kill a target cancer cell that expresses the first target cell surface antigen, but not the second target cell surface antigen, on its cell surface; and b) is inhibited from killing a non-cancerous cell if the non-cancerous cell expresses both the first target cell surface antigen and the second target cell surface antigen on its cell surface; in these cases, the genetically modified cytotoxic immune cell must express both a first antigen-triggered polypeptide that specifically binds the first target antigen of the target antigen pair and a second triggered polypeptide that specifically binds the second antigen of the target antigen pair. Non-limiting examples of 2-input AND-NOT gates (AND-NOT gates based on 2 target antigens) are depicted schematically in FIG. 7B.

As an example, in some cases, the first antigen-triggered polypeptide is a CAR, and the second antigen-triggered polypeptide is an iCAR. Binding of the iCAR to the second antigen (present on the surface of a non-cancerous cell, but not on the surface of a target cancer cell) of a target antigen pair inhibits T-cell activation mediated by activation of the CAR upon binding to the first antigen (present on the surface of the target cancer cell and on the surface of the non-cancerous cell) of the target antigen pair. As another example, in some cases, the first antigen-triggered polypeptide is a TCR, and the second antigen-triggered polypeptide is an iCAR. Binding of the iCAR to the second antigen (present on the surface of a non-cancerous cell, but not on the surface of a target cancer cell) of a target antigen pair blocks or reduces T-cell activation mediated by activation of the TCR upon binding to the first antigen (present on the surface of the target cancer cell and on the surface of the non-cancerous cell) of the target antigen pair.

The above provides examples of AND-NOT gates where the inhibitory component is an iCAR; however, as will be readily understood, the inhibitory components of combinatorial antigen gates having “NOT” functionality are not so limited and may generally include any polypeptide configured to inhibit an activity, e.g., an activity induced by binding of a first activating antigen in an AND-NOT gate, including where such inhibition is conferred through the presence of an inhibitory domain. Inhibitory components of combinatorial antigen gates having “NOT” functionality may be specific for an antigen present on a non-target cell, including e.g., where such antigen is absent or present in low amounts on the surface of a target cell.

As another example, in some cases, the first antigen-triggered polypeptide is a CAR, and the second antigen-triggered polypeptide is a BTTS comprising an intracellular domain that, when released upon activation of the BTTS by binding to the second target antigen, induces expression of an intracellular inhibitor that inhibits T-cell activation mediated by activation of the CAR upon binding to the first antigen (present on the surface of the target cancer cell and on the surface of the non-cancerous cell) of the target antigen pair. As another example, in some cases, the first antigen-triggered polypeptide is a TCR, and the second antigen-triggered polypeptide is a BTTS comprising an intracellular domain that, when released upon activation of the BTTS by binding to the second target antigen, induces expression of an intracellular inhibitor that inhibits T-cell activation mediated by activation of the TCR upon binding to the first antigen (present on the surface of the target cancer cell and on the surface of the non-cancerous cell) of the target antigen pair.

As another example, in some cases, the first antigen-triggered polypeptide is a CAR, and the second antigen-triggered polypeptide is a BTTS comprising an intracellular domain that, when released upon activation of the BTTS by binding to the second target antigen, induces expression of an extracellular inhibitor that inhibits T-cell activation mediated by activation of the CAR upon binding to the first antigen (present on the surface of the target cancer cell and on the surface of the non-cancerous cell) of the target antigen pair. As another example, in some cases, the first antigen-triggered polypeptide is a TCR, and the second antigen-triggered polypeptide is a BTTS comprising an intracellular domain that, when released upon activation of the BTTS by binding to the second target antigen, induces expression of an extracellular inhibitor that inhibits T-cell activation mediated by activation of the TCR upon binding to the first antigen (present on the surface of the target cancer cell and on the surface of the non-cancerous cell) of the target antigen pair.

### Antigen Triplets

As noted above, in some cases, a genetically modified cytotoxic immune cell of the present disclosure, or a system of the present disclosure, can include an antigen-triggered polypeptide (or a nucleic acid comprising a nucleotide sequence encoding the same) that specifically binds a third target antigen present on the surface of a cancer cell. Examples of such third target antigens are listed in Table 1.

Where a third antigen is included in the target antigen combination, the third antigen may be associated with the same cancer cell type as a target antigen pair

In some instances, a particular antigen may be employed in an antigen combination, including combinations having two antigens, combinations having three antigens, etc., for treating a cancer other than the exemplary cancer with which it is identified. For example, although Table 1 identifies exemplary cancers for each antigen, use of the antigen in antigen combinations will not be limited to the specifically identified cancer(s) and the particular antigen may be employed in antigen combinations for the treatment of other cancers besides those specifically listed as exemplary.

Useful three antigen combinations may include a clinical antigen. For example, in some instances, a useful three antigen combination may include a clinical antigen and two or more antigens that provide AND NOT functionality, including e.g., the combinations including a clinical antigen and two tissue specific AND NOT antigens (e.g., brain tissue and cardiac tissue) as described in more detail below. In some cases, useful three antigen combinations may not include a clinical antigen. Useful three antigen combinations may include various logic combinations including but not limited to e.g., antigen 1 AND antigen 2 AND antigen 3, antigen 1 AND antigen 2 AND NOT antigen 3, antigen 1 AND NOT antigen 2 AND NOT antigen 3, and the like. In some instances, the logic of a three antigen combination may be complex where “complex” logic, as used herein, refers to combinations having multiple positive prediction nodes in the associated tree or, e.g., where the logic contains one or more OR propositions.

In some cases, useful three antigen combinations include an antigen combination that includes a clinical antigen in combination with two or more tissue antigens (e.g., a first antigen expressed in normal tissue, e.g., normal brain tissue, and a second antigen expressed in normal tissue, e.g., normal cardiac tissue) in a double AND NOT gate (i.e., clinical antigen AND NOT normal tissue antigen 1 AND NOT normal tissue antigen 2).

### Antigen-Triggered Polypeptides

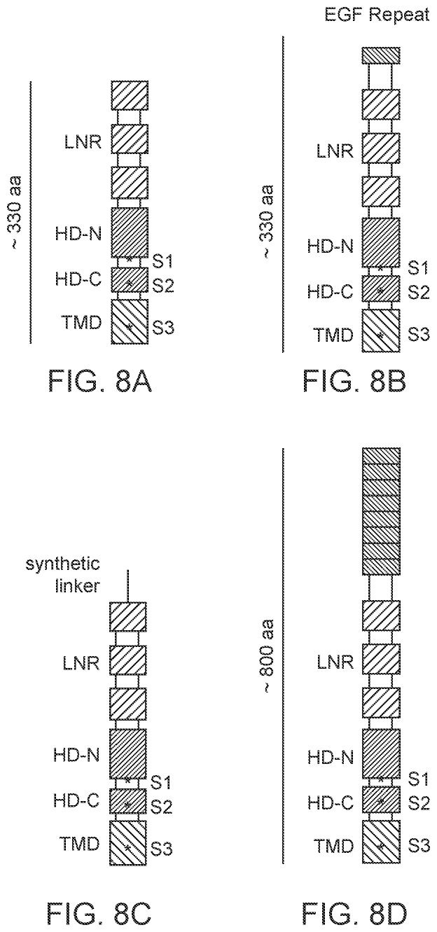

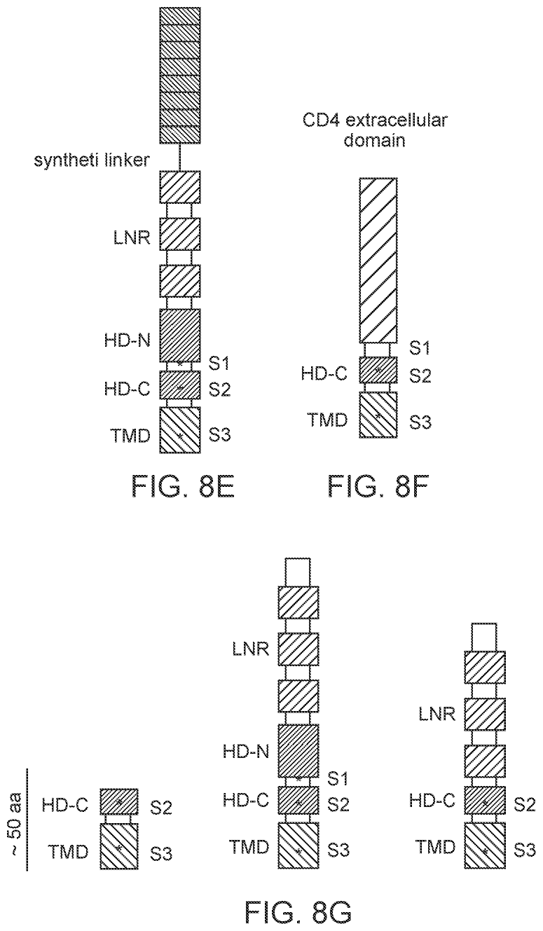

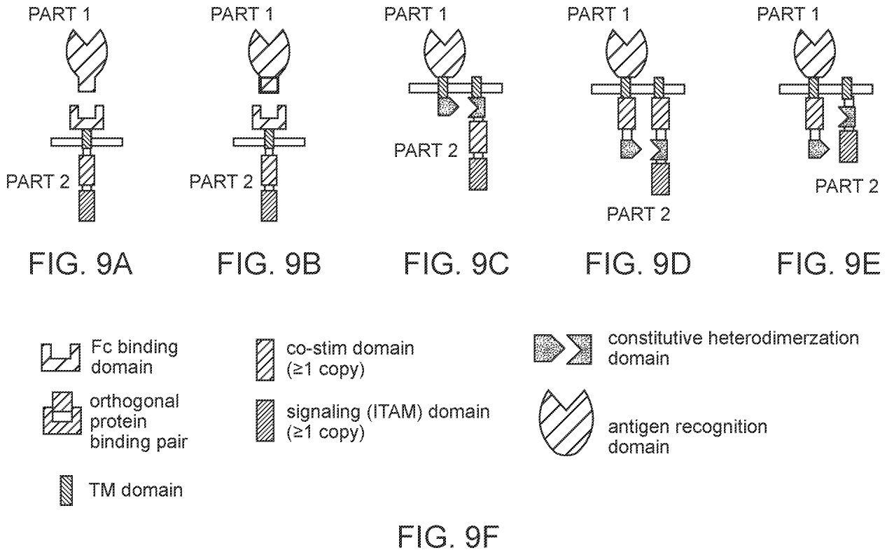

As noted above, an antigen-triggered polypeptide can be a binding triggered transcriptional switch (BTTS); a CAR; or a TCR. A CAR can be an ON-switch (“split”) CAR, a single-chain CAR, an iCAR, etc. Schematic depictions of examples of antigen-triggered polypeptides are provided in FIGS. 8A-8G and FIGS. 9A-9F.

### Binding-Triggered Transcriptional Switches

As noted above, in some cases an antigen-triggered polypeptide produced in a genetically modified immune cell of the present disclosure, or present in a system of the present disclosure, or encoded by a nucleotide sequence in a nucleic acid present in a system of the present disclosure, is a binding triggered transcriptional switch (BTTS).

As used herein, a “binding-triggered transcriptional switch” (BTTS) generally refers to a synthetic modular polypeptide or system of interacting polypeptides having an extracellular domain that includes a first member of a specific binding pair that binds a binding partner (i.e., the second member of the specific binding pair; e.g., an antigen), a binding-transducer and an intracellular domain. Upon binding of the second member of the specific binding pair to the BTTS the binding signal is transduced to the intracellular domain such that the intracellular domain becomes activated and performs some function within the cell that it does not perform in the absence of the binding signal. Certain BTTS's are described in e.g., PCT Pub. No. WO 2016/138034 as well as U.S. Pat. Nos. 9,670,281 and 9,834,608; the disclosures of which are incorporated herein by reference in their entirety.

The specific binding member of the extracellular domain generally determines the specificity of the BTTS. In some instances, a BTTS may be referred according to its specificity as determined based on its specific binding member. For example, a specific binding member having binding partner “X” may be referred to as an X-BTTS or an anti-X BTTS.

A BTTS useful in the cells, systems, methods, etc., of the present disclosure may make use of a member of a specific binding pair, i.e., specific binding member, and thus, the BTTS may be specific for an antigen as described herein. Useful specific binding members include but not limited to e.g., antigen-antibody pairs, ligand receptor pairs, scaffold protein pairs, etc., including those specific for an antigen described herein.

In some instances, the specific binding member may be an antibody and its binding partner may be an antigen to which the antibody specifically binds. In some instances, the specific binding member may be a receptor and its binding partner may be a ligand to which the receptor specifically binds. In some instances, the specific binding member may be a scaffold protein and its binding partner may be a protein to which the scaffold protein specifically binds.

Useful specific binding pairs include those specific for an antigen, including those antigens described herein. For simplicity, regardless of the actual nature of the binding pair (i.e., antigen/antibody, receptor/ligand, etc.), the member of the binding pair attached to the BTTS will be referred to herein as an antigen binding domain and the member to which it binds will be referred to as an antigen herein (i.e., regardless of whether such a molecule would otherwise be considered an “antigen” in the conventional sense). However, one of ordinary skill will readily understand that descriptions of antigen binding domain-antigen interactions can be substituted with ligand/receptor, scaffold/binding partner pair where desired as appropriate.

In some cases, the specific binding member is an antibody. The antibody can be any antigen-binding antibody-based polypeptide, a wide variety of which are known in the art. In some instances, the specific binding member is or includes a monoclonal antibody, a single chain Fv (scFv), a Fab, etc. Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

Where the specific binding member is an antibody-based binding member, the BTTS can be activated in the presence of a binding partner to the antibody-based binding member, including e.g., an antigen specifically bound by the antibody-based binding member. In some instances, antibody-based binding member may be defined, as is commonly done in the relevant art, based on the antigen bound by the antibody-based binding member, including e.g., where the antibody-based binding member is described as an “anti-” antigen antibody, e.g., an anti-CD19 antibody. Accordingly, antibody-based binding members suitable for inclusion in a BTTS or an antigen-specific therapeutic of the present methods can have a variety of antigen-binding specificities.

The components of BTTS's, employed in the described cells, systems, methods, etc., and the arrangement of the components of the switch relative to one another will vary depending on many factors including but not limited to e.g., the desired antigen, the activity of the intracellular domain, the overall function of the BTTS, the broader arrangement of a system comprising the BTTS, etc. The first binding member may include but is not limited to e.g., those agents that bind an antigen described herein. The intracellular domain may include but is not limited e.g., those intracellular domains that activate or repress transcription at a regulatory sequence, e.g., to induce or inhibit expression of a downstream component such as an antigen-triggered polypeptide (e.g., a second antigen-triggered polypeptide).

The binding transducer of BTTS's will also vary depending on the desired method of transduction of the binding signal. Generally, binding transducers may include those polypeptides and/or domains of polypeptides that transduce an extracellular signal to intracellular signaling e.g., as performed by the receptors of various signal transduction pathways. Transduction of a binding signal may be achieved through various mechanisms including but not limited to e.g., binding-induced proteolytic cleavage, binding-induced phosphorylation, binding-induced conformational change, etc. In some instances, a binding-transducer may contain a ligand-inducible proteolytic cleavage site such that upon binding the binding-signal is transduced by cleavage of the BTTS, e.g., to liberate an intracellular domain. For example, in some instances, a BTTS may include a Notch derived cleavable binding transducer, such as, e.g., a chimeric notch receptor polypeptide (e.g., a synNotch polypeptide) as described herein.

In other instances, the binding signal may be transduced in the absence of inducible proteolytic cleavage. Any signal transduction component or components of a signaling transduction pathway may find use in a BTTS whether or not proteolytic cleavage is necessary for signal propagation. For example, in some instances, a phosphorylation-based binding transducer, including but not limited to e.g., one or more signal transduction components of the Jak-Stat pathway, may find use in a non-proteolytic BTTS.

For simplicity, BTTS's, including but not limited to chimeric notch receptor polypeptides, are described primarily as single polypeptide chains. However, BTTS's, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTS's that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety.

Useful BTTS's that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105(1):64-9); the disclosure of which is incorporated herein by reference in its entirety.

Useful BTTS's that may be employed in the subject methods include, but are not limited to von Willebrand Factor (vWF) cleavage domain-based BTTS's, such as but not limited to e.g., those containing an unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTS's are described in Langridge & Struhl (Cell (2017) 171(6):1383-1396); the disclosure of which is incorporated herein by reference in its entirety.

Useful BTTS's that may be employed in the subject methods include, but are not limited to chimeric Notch receptor polypeptides, such as but not limited to e.g., synNotch polypeptides (also referred to as “synNotch receptors”), non-limiting examples of which are described in PCT Pub. No. WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91; the disclosures of which are incorporated herein by reference in their entirety.

SynNotch polypeptides are generally proteolytically cleavable chimeric polypeptides that generally include: a) an extracellular domain comprising a specific binding member; b) a proteolytically cleavable Notch receptor polypeptide comprising one or more proteolytic cleavage sites; and c) an intracellular domain. Binding of the specific binding member by its binding partner generally induces cleavage of the synNotch at the one or more proteolytic cleavage sites, thereby releasing the intracellular domain. In some instances, the instant methods may include where release of the intracellular domain triggers (i.e., induces) the production of an encoded payload, the encoding nucleic acid sequence of which is contained within the cell. Depending on the particular context, the produced payload is then generally expressed on the cell surface or secreted. SynNotch polypeptides generally include at least one sequence that is heterologous to the Notch receptor polypeptide (i.e., is not derived from a Notch receptor), including e.g., where the extracellular domain is heterologous, where the intracellular domain is heterologous, where both the extracellular domain and the intracellular domain are heterologous to the Notch receptor, etc.

Useful synNotch BTTS's will vary in the domains employed and the architecture of such domains. SynNotch polypeptides will generally include a Notch receptor polypeptide that includes one or more ligand-inducible proteolytic cleavage sites. The length of Notch receptor polypeptides will vary and may range in length from about 50 amino acids or less to about 1000 amino acids or more.

In some cases, the Notch receptor polypeptide present in a synNotch polypeptide has a length of from 50 amino acids (aa) to 1000 aa, e.g., from 50 aa to 75 aa, from 75 aa to 100 aa, from 100 aa to 150 aa, from 150 aa to 200 aa, from 200 aa to 250 aa, from 250 a to 300 aa, from 300 aa to 350 aa, from 350 aa to 400 aa, from 400 aa to 450 aa, from 450 aa to 500 aa, from 500 aa to 550 aa, from 550 aa to 600 aa, from 600 aa to 650 aa, from 650 aa to 700 aa, from 700 aa to 750 aa, from 750 aa to 800 aa, from 800 aa to 850 aa, from 850 aa to 900 aa, from 900 aa to 950 aa, or from 950 aa to 1000 aa. In some cases, the Notch receptor polypeptide present in a synNotch polypeptide has a length of from 300 aa to 400 aa, from 300 aa to 350 aa, from 300 aa to 325 aa, from 350 aa to 400 aa, from 750 aa to 850 aa, from 50 aa to 75 aa. In some cases, the Notch receptor polypeptide has a length of from 310 aa to 320 aa, e.g., 310 aa, 311 aa, 312 aa, 313 aa, 314 aa, 315 aa, 316 aa, 317 aa, 318 aa, 319 aa, or 320 aa. In some cases, the Notch receptor polypeptide has a length of 315 aa. In some cases, the Notch receptor polypeptide has a length of from 360 aa to 370 aa, e.g., 360 aa, 361 aa, 362 aa, 363 aa 364 aa, 365 aa, 366 aa, 367 aa, 368 aa, 369 aa, or 370 aa. In some cases, the Notch receptor polypeptide has a length of 367 aa.

In some cases, a Notch receptor polypeptide comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a Notch receptor. In some instances, the Notch regulatory region of a Notch receptor polypeptide is a mammalian Notch regulatory region, including but not limited to e.g., a mouse Notch (e.g., mouse Notch1, mouse Notch2, mouse Notch3 or mouse Notch4) regulatory region, a rat Notch regulatory region (e.g., rat Notch1, rat Notch2 or rat Notch3), a human Notch regulatory region (e.g., human Notch1, human Notch2, human Notch3 or human Notch4), and the like or a Notch regulatory region derived from a mammalian Notch regulatory region and having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a mammalian Notch regulatory region of a mammalian Notch receptor amino acid sequence.

Subject Notch regulatory regions may include or exclude various components (e.g., domains, cleavage sites, etc.) thereof. Examples of such components of Notch regulatory regions that may be present or absent in whole or in part, as appropriate, include e.g., one or more EGF-like repeat domains, one or more Lin12/Notch repeat domains, one or more heterodimerization domains (e.g., HD-N or HD-C), a transmembrane domain, one or more proteolytic cleavage sites (e.g., a furin-like protease site (e.g., an S1 site), an ADAM-family protease site (e.g., an S2 site) and/or a gamma-secretase protease site (e.g., an S3 site)), and the like. Notch receptor polypeptides may, in some instances, exclude all or a portion of one or more Notch extracellular domains, including e.g., Notch-ligand binding domains such as Delta-binding domains. Notch receptor polypeptides may, in some instances, include one or more non-functional versions of one or more Notch extracellular domains, including e.g., Notch-ligand binding domains such as Delta-binding domains. Notch receptor polypeptides may, in some instances, exclude all or a portion of one or more Notch intracellular domains, including e.g., Notch Rbp-associated molecule domains (i.e., RAM domains), Notch Ankyrin repeat domains, Notch transactivation domains, Notch PEST domains, and the like. Notch receptor polypeptides may, in some instances, include one or more non-functional versions of one or more Notch intracellular domains, including e.g., non-functional Notch Rbp-associated molecule domains (i.e., RAM domains), non-functional Notch Ankyrin repeat domains, non-functional Notch transactivation domains, non-functional Notch PEST domains, and the like.

Non-limiting examples of particular synNotch BTTS's, the domains thereof, and suitable domain arrangements are described in PCT Pub. Nos. WO 2016/138034, WO 2017/193059, WO 2018/039247 and U.S. Pat. Nos. 9,670,281 and 9,834,608; the disclosures of which are incorporated herein by reference in their entirety.

Domains of a useful BTTS, e.g., the extracellular domain, the binding-transducer domain, the intracellular domain, etc., may be joined directly, i.e., with no intervening amino acid residues or may include a peptide linker that joins two domains. Peptide linkers may be synthetic or naturally derived including e.g., a fragment of a naturally occurring polypeptide.

A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

In some instances, a BTTS may have an extracellular domain that includes a first member of a specific binding pair that binds a second member of the specific binding pair, wherein the extracellular domain does not include any additional first or second member of a second specific binding pair. For example, in some instances, a BTTS may have an extracellular domain that includes a first antigen-binding domain that binds an antigen, wherein the extracellular domain does not include any additional antigen-binding domains and does not bind any other antigens. A subject BTTS may, in some instances, include only a single extracellular domain. Accordingly, an employed BTTS may be specific for a single antigen and only specific for the single antigen. Such, BTTS's may be referred to as a “single antigen BTTS”.

BTTS's specific for a single antigen may be monovalent or multivalent (e.g., bivalent, trivalent, etc.) for the antigen. For example, in some instances, a monovalent BTTS may be employed that includes an antigen binding domain (e.g., a single antigen binding domain) for binding a single molecule of antigen. In some instances, a multivalent BTTS may be employed that includes an antigen binding domain or multiple antigen binding domains (e.g., 1, 2, 3, 4, 5, 6, etc. antigen binding domains) for binding multiple molecules of antigen.

In some instances, a BTTS may have an extracellular domain that includes the first or second members of two or more specific binding pairs. For example, in some instances, a BTTS may have an extracellular domain that includes a first antigen-binding domain and a second antigen-binding domain that are different such that the extracellular domain is specific for two different antigens. In some instances, a BTTS may have two or more extracellular domains that each includes the first or second members of two different specific binding pairs. For example, in some instances, a BTTS may have a first extracellular domain that includes a first antigen-binding domain and a second extracellular domain that includes a second antigen-binding domain where the two different antigen binding domains are each specific for a different antigen. As such, the BTTS may be specific for two different antigens.

A BTTS specific for two or more different antigens, containing either two extracellular domains or one extracellular domain specific for two different antigens, may be configured such that the binding of either antigen to the BTTS is sufficient to trigger activation of the BTTS, e.g., proteolytic cleavage of a cleavage domain of the BTTS, e.g., releasing an intracellular domain of the BTTS. Such a BTTS, capable of being triggered by any of two or more antigens, may find use as a component of a logic gate containing OR functionality. In some instances, a BTTS specific for two different antigens may be referred to as a “two-headed BTTS”. BTTS's specific for multiple antigens will not be limited to only two antigens and may, e.g., be specific for and/or triggered by more than two antigens, including e.g., three or more, four or more, five or more, etc.

As summarized above, antigen binding domains of BTTS's may be substituted, amended or exchanged as desired. For example, an antigen binding domain of any antigen specific molecule, such as an antibody, may be employed as the antigen binding domain of a BTTS described herein. Correspondingly, an antigen binding domain described above as used in a CAR may be employed in other contexts, such as e.g., in a BTTS as described. As such, disclosure of any agent targeted to a specific antigen in any context herein would be understood to constitute a disclosure of the use of an antigen binding domain in any other antigen-specific polypeptide described herein as well.

### CARs

As noted above, in some cases an antigen-triggered polypeptide produced in a genetically modified immune cell of the present disclosure, or present in a system of the present disclosure, or encoded by a nucleotide sequence in a nucleic acid present in a system of the present disclosure, is a chimeric antigen receptor. Schematic depictions of split CARs are provided in FIGS. 9A-9F.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

Spit CAR may be extracellularly split or intracellularly split and may or may not be conditionally heterodimerizable. For example, split CAR systems that are not conditionally heterodimerizable may contain a constitutive heterodimerization domain or other binding pair (e.g., a Fc binding pair or other orthogonal binding pair) that does not depend on the presence of one or more additional molecules for the heterodimerization that results in the formation of an active CAR from assembly of the split portions.

In some instances, an extracellularly split CAR may be split extracellularly at the antigen binding domain into two parts including e.g., where the first part of the split CAR contains an extracellular Fc binding domain that specifically binds to second part of the split CAR that contains the antigen recognition domain.

In some instances, an extracellularly split CAR may be split extracellularly at the antigen binding domain into two parts including e.g., where the first part of the split CAR contains an first part of an orthogonal protein binding pair that specifically binds to the second part of the orthogonal protein binding pair that is contained in the second part of the split CAR that contains the antigen recognition domain.

In some instances, an intracellularly split CAR may be split intracellularly proximal to the transmembrane domain into two parts including e.g., where the first part of the split CAR includes the antigen recognition domain, a transmembrane domain and an intracellular first portion of a constitutive heterodimerization domain and the second part of the split CAR includes a transmembrane domain, the second portion of the constitutive heterodimerization domain proximal to the transmembrane domain, one or more co-stimulatory domains and one or more signaling domains (e.g., ITAM domains).

In some instances, an intracellularly split CAR may be split into two parts intracellularly proximal to an intracellular domain or between two intracellular domains including e.g., where the first part of the split CAR includes the antigen recognition domain, a transmembrane domain, one or more co-stimulatory domains and an intracellular first portion of a constitutive heterodimerization domain and the second part of the split CAR includes a transmembrane domain, one or more co-stimulatory domains, one or more signaling domains (e.g., ITAM domains) and the second portion of the constitutive heterodimerization domain between the one or more co-stimulatory domains and the one or more signaling domains.

In some instances, an intracellularly split CAR may be split into two parts intracellularly between intracellular domains including e.g., where the first part of the split CAR includes the antigen recognition domain, a transmembrane domain, one or more co-stimulatory domains and an intracellular first portion of a constitutive heterodimerization domain proximal to the intracellular terminus of the first part of the split CAR and the second part of the split CAR includes a transmembrane domain, one or more signaling domains (e.g., ITAM domains) and the second portion of the constitutive heterodimerization domain between the transmembrane domain and the one or more signaling domains.

An inhibitory CAR (iCAR) expressed on an immunoresponsive cell specifically binds to an antigen, whereupon binding its antigen the iCAR inhibits the immunoresponsive cell. By “inhibits an immunoresponsive cell” or “suppresses an immunoresponsive cell” is meant induction of signal transduction or changes in protein expression in the cell resulting in suppression of an immune response (e.g., decrease in cytokine production).

Generally, but not exclusively, an iCAR is employed as a component of a bispecific CAR system where the activity of an immunostimulatory CAR (e.g., a CAR or CAR variant) is repressed by the iCAR upon binding of the iCAR to its antigen. An iCAR will generally include an extracellular domain that binds an antigen; a transmembrane domain operably linked to the extracellular domain; and an intracellular domain that activates intracellular signaling to decrease an immune response, the intracellular domain operably linked to the transmembrane domain. In some embodiments, the intracellular signaling domain is selected from the group consisting of a CTLA-4 polypeptide, a PD-1 polypeptide, a LAG-3 polypeptide, a 2B4 polypeptide, and a BTLA polypeptide. In certain embodiments, the transmembrane domain is selected from the group consisting of a CD4 polypeptide, a CD8 polypeptide, a CTLA-4 polypeptide, a PD-1 polypeptide, a LAG-3 polypeptide, a 2B4 polypeptide, and a BTLA polypeptide. In some instances, an iCAR, as described herein, may be or may be derived from one or more of the iCARs described in U.S. Patent Application Publication No. 20150376296, the disclosure of which is incorporated herein by reference in its entirety.

Any convenient extracellular binding domain (i.e., antigen binding domain) may find use in an iCAR including but not limited to e.g., a Fab, scFv, a monovalent or polyvalent ligand, etc., provided the domain is sufficient for specific binding of the iCAR to its antigen. In the contexts of therapy, the antigen binding domain of an iCAR will generally bind a healthy cell antigen in order to repress an immune response that may be otherwise triggered by presentation of a target antigen on the surface of a healthy cell. For example, in the contexts of cancer therapy, the antigen binding domain of an iCAR will generally bind a non-tumor or healthy cell antigen.

In certain instances, an antigen to which the extracellular domain of an iCAR binds may be an antigen listed in Table 1, which antigen is described as the “NOT” portion of an antigen logic gate.

### TCRs

As noted above, in some cases an antigen-triggered polypeptide produced in a genetically modified immune cell of the present disclosure, or present in a system of the present disclosure, or encoded by a nucleotide sequence in a nucleic acid present in a system of the present disclosure, is a T-cell receptor (TCR).

A TCR generally includes an alpha chain and a beta chain; and recognizes antigen when presented by a major histocompatibility complex. In some cases, the TCR is an engineered TCR. Any engineered TCR having immune cell activation function can be induced using a method of the present disclosure. Such TCRs include, e.g., antigen-specific TCRs, Monoclonal TCRs (MTCRs), Single chain MTCRs, High Affinity CDR2 Mutant TCRs, CD1-binding MTCRs, High Affinity NY-ESO TCRs, VYG HLA-A24 Telomerase TCRs, including e.g., those described in PCT Pub Nos. WO 2003/020763, WO 2004/033685, WO 2004/044004, WO 2005/114215, WO 2006/000830, WO 2008/038002, WO 2008/039818, WO 2004/074322, WO 2005/113595, WO 2006/125962; Strommes et al. Immunol Rev. 2014; 257(1):145-64; Schmitt et al. Blood. 2013; 122(3):348-56; Chapuls et al. Sci Transl Med. 2013; 5(174):174ra27; Thaxton et al. Hum Vaccin Immunother. 2014; 10(11):3313-21 (PMID:25483644); Gschweng et al. Immunol Rev. 2014; 257(1):237-49 (PMID:24329801); Hinrichs et al. Immunol Rev. 2014; 257(1):56-71 (PMID:24329789); Zoete et al. Front Immunol. 2013; 4:268 (PMID:24062738); Marr et al. Clin Exp Immunol. 2012; 167(2):216-25 (PMID:22235997); Zhang et al. Adv Drug Deliv Rev. 2012; 64(8):756-62 (PMID:22178904); Chhabra et al. Scientific World Journal. 2011; 11:121-9 (PMID:21218269); Boulter et al. Clin Exp Immunol. 2005; 142(3):454-60 (PMID:16297157); Sami et al. Protein Eng Des Sel. 2007; 20(8):397-403; Boulter et al. Protein Eng. 2003; 16(9):707-11; Ashfield et al. IDrugs. 2006; 9(8):554-9; Li et al. Nat Biotechnol. 2005; 23(3):349-54; Dunn et al. Protein Sci. 2006; 15(4):710-21; Liddy et al. Mol Biotechnol. 2010; 45(2); Liddy et al. Nat Med. 2012; 18(6):980-7; Oates, et al. Oncoimmunology. 2013; 2(2):e22891; McCormack, et al. Cancer Immunol Immunother. 2013 April; 62(4):773-85; Bossi et al. Cancer Immunol Immunother. 2014; 63(5):437-48 and Oates, et al. Mol Immunol. 2015 October; 67(2 Pt A):67-74; the disclosures of which are incorporated herein by reference in their entirety.

### Antigen-Binding Inhibitory Polypeptides

In some cases, an antigen-triggered polypeptide produced in a genetically modified immune cell of the present disclosure, or present in a system of the present disclosure, or encoded by a nucleotide sequence in a nucleic acid present in a system of the present disclosure, may be an inhibitory polypeptide. The term “antigen-binding inhibitory polypeptide”, as used herein, will generally describe a polypeptide, specific for an antigen, that upon binding the antigen inhibits the activity of a second polypeptide (e.g., an activating antigen-specific polypeptide, such as a CAR or TCR or other synthetic stimulatory immune cell receptor) and/or an activity of a cell (e.g., immune activation). iCARs, as described above, are an example of an antigen-binding inhibitory polypeptide; however, the term antigen-binding inhibitory polypeptide is not so limited.

Antigen-binding inhibitory polypeptides will vary and will generally function to mediate repression of an activated or activatable immune cell, including e.g., an immune cell expressing a stimulatory receptor, such as a CAR or TCR. An antigen-binding inhibitory polypeptide will include an inhibitory domain that functions to repress immune cell activation, including e.g., immune cell activation attributed to a stimulatory receptor, such as a CAR or TCR. Domains useful as inhibitory domains will vary depending on the particular context of immune cell activation and repression, including e.g., the particular type of activated cell to be repressed and the desired degree of repression. Exemplary non-limited examples of inhibitory domains include but are not limited to domains and motifs thereof derived from immune receptors including, e.g., co-inhibitory molecules, immune checkpoint molecules, immune tolerance molecules, and the like.

Suitable intracellular inhibitory domains may be any functional unit of a polypeptide as short as a 3 amino acid linear motif and as long as an entire protein, where size of the inhibitory domain is restricted only in that the domain must be sufficiently large as to retain its function and sufficiently small so as to be compatible with the other components of the polypeptide. Accordingly, an inhibitory domain may range in size from 3 amino acids in length to 1000 amino acids or more and, in some instances, can have a length of from about 30 amino acids to about 70 amino acids (aa), e.g., an inhibitory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, an inhibitory domain can have a length of from about 70 aa to about 100 aa, from about 100 aa to about 200 aa, or greater than 200 aa.

In some instances, “co-inhibitory domains” find use in the subject polypeptides. Such co-inhibitory domains are generally polypeptides derived from receptors. Co-inhibition generally refers to the secondary inhibition of primary antigen-specific activation mechanisms which prevents co-stimulation. Co-inhibition, e.g., T cell co-inhibition, and the factors involved have been described in Chen & Flies. Nat Rev Immunol (2013) 13(4):227-42 and Thaventhiran et al. J Clin Cell Immunol (2012) S12, the disclosures of which are incorporated herein by reference in their entirety. In some embodiments, co-inhibitory domains homodimerize. In some instances, useful co-inhibitory domains have been modified to constitutively dimerize, including constitutively homodimerize. A subject co-inhibitory domain can be an intracellular portion of a transmembrane protein (i.e., the co-inhibitory domain can be derived from a transmembrane protein). Non-limiting examples of suitable co-inhibitory polypeptides include, but are not limited to, CTLA-4 and PD-1. In some instances, a co-inhibitory domain, e.g., as used in a subject polypeptide may include a co-inhibitory domain selected from PD-1, CTLA4, HPK1, SHP1, SHP2, Sts1 and Csk. In some instances, a co-inhibitory domain of subject polypeptide comprises an amino acid sequence having at least about 75%, at least about 80%, at least about 85%, at least about 90%, at least about 95%, at least about 98%, or 100% amino acid sequence identity to a co-inhibitory domain as described herein.

In some instances, an antigen-binding inhibitory polypeptide may include a domain of a dimerization pair, such as e.g., a synthetic immune cell receptor (ICR) repressor useful as a component of a heteromeric, conditionally repressible synthetic ICR. Components of a heteromeric, conditionally repressible synthetic ICR may include a synthetic stimulatory ICR and a synthetic ICR repressor, where e.g., the synthetic stimulatory ICR and the synthetic ICR repressor specifically bind the antigens of an antigen pair described herein. Heteromeric, conditionally repressible synthetic ICRs, and components thereof, are described in PCT Application No. PCT/US2016/062612; the disclosure of which is incorporated herein by reference in its entirety.

### Polyspecific-Immune-Inducing Polypeptides

The present disclosure includes polyspecific-immune-inducing polypeptides having specificity for the antigens of an antigen logic pair described herein. By “polyspecific-immune-inducing polypeptide” is generally meant a single polypeptide having specificity for two or more distinct antigens that, when bound to one or more of the antigens to which the polyspecific-immune-inducing polypeptide binds, induces an immune response in a subject. The multi-specificity of a polyspecific-immune-inducing polypeptide (PIIP) may vary and may include but is not limited to e.g., bispecific-immune-inducing polypeptides, trispecific-immune-inducing polypeptides, and the like. For example, in some instances, a PIIP may be a bispecific-immune-inducing polypeptide that is a single polypeptide having specificity for two distinct antigens. Such a bispecific PIIP, when administered and bound to one or both of the antigens, may induce an immune response in a subject. In some instances, an immune response induced by antigen binding of a PIIP may only be induced when the PIIP is bound to all of the antigens (e.g., both of the antigens in the case of a bispecific PIIP) to which the PIIP is specific. In some instances, an immune response induced by antigen binding of a PIIP may be induced when the PIIP is bound to less than all of the antigens (e.g., one of the two antigens in the case of a bispecific PIIP) to which the PIIP is specific.

As such, the described PIIPs of the present disclosure may provide for AND and/or OR logic gate functionality. In some instances, a PIIP of the present disclosure may induce an immune response to a cell expressing one antigen to which the PIIP is specific. In some instances, a PIIP of the present disclosure may induce an immune response to a cell expressing all antigens to which the PIIP is specific. In some instances, the immune response induced by a PIIP bound to two or more, including e.g., all, of the antigens to which the PIIP is specific is enhanced relative to any immune response induced by the PIIP due to binding one antigen to which the PIIP is specific. The enhancement due to multiple antigen binding may vary and may be at least 10% greater, including but not limited to e.g., at least 15% greater, at least 20% greater, at least 30% greater, at least 40% greater, at least 50% greater, at least 60% greater, at least 70% greater, at least 80% greater, at least 90% greater, at least 2-fold greater, at least 3-fold greater, etc., than single antigen binding. In some instances, an enhanced immune response due to binding two or more, including e.g., all, of the antigens to which the PIIP is specific is synergistically increased. For example, in some instances, the enhanced immune response induced by a PIIP of the present disclosure binding two or more, including e.g., all, of the antigens to which the PIIP is specific may be greater than the mere additive effect of comparable immune-inducing polypeptides directed to each antigen individually. Immune responses, including enhanced immune responses, resulting from PIIP antigen binding may be evaluated using any convenient and appropriate method, including but not limited to e.g., methods measuring immune activation, methods measuring immune-mediated toxicity, and the like.

In some instances, antigen combinations useful in a PIIP of the present disclosure may include any of the combinations listed in Table 1. The individual antigens, including representative amino acid sequences thereof may be found in Genbank. Antigen binding domains of the subject PIIP of the present disclosure may, in some instances, specifically bind an antigen polypeptide comprising an amino acid sequence having at least 85%, at least 90%, at least 95%, at least 98%, at least 99%, or 100%, amino acid sequence identity to an amino acid sequence of an antigen set forth in PCT Pub. WO 2017/192059 A1.

Useful PIIPs of the present disclosure may be specific for a target cancer cell. For example, a PIIP of the present disclosure may employ an antigen combination to target a cancer cell, where the targeted cancer may correspond with the cancer identified in Table 1 for the particular antigen combination employed.

The PIIP of the present disclosure will generally include two or more antigen binding domains and at least one portion that functions to induce an immune response. Useful immune-inducing portions of the subject PIIPs will vary and may be derived from various immune-inducing molecules, such as but not limited to e.g., chimeric antigen receptors (CARs), T cell receptors (TCRs), antibodies, and the like. Correspondingly, in some instances, a PIIP of the present disclosure may be a polyspecific CAR, including but not limited to e.g., a bispecific CAR. In some instances, a PIIP of the present disclosure may be a polyspecific TCR, including but not limited to e.g., a bispecific TCR. In some instances, a PIIP of the present disclosure may be a polyspecific antibody, including but not limited to e.g., a bispecific antibody.

The immune response induced by a PIIP bound to antigens of an antigen combination described herein may vary and may depend on the nature of immune-inducing polypeptide employed. For example, in some instances, a PIIP of the present disclosure may be a polyspecific chimeric antigen receptor (CAR) and the immune response induced may be a CAR-specific immune response. CAR-specific immune responses include an immune response induced by the CAR binding its target(s) and signaling through an intracellular signaling domain of the CAR. The results of a CAR-specific immune response may vary and may include e.g., immune activation of an immune cell expressing the CAR, increased proliferation of an immune cell expressing the CAR, secretion and/or expression (including increased secretion and/or expression) of an immune molecule (e.g., a cytokine) by an immune cell expressing the CAR, increased cytotoxic activity of an immune cell expressing the CAR, increased killing of a cell to which the CAR is targeted, etc.

In some instances, a polyspecific CAR of the present disclosure may be derived from a previously investigated or otherwise available CAR, e.g., by replacing or amending the antigen-binding portion of the previously investigated or otherwise available CAR with one or more antigen binding domains such that the resulting polyspecific CAR is specific for the desired antigen combination, including e.g., an antigen combination described herein. Put another way, in some instances, an existing CAR may be reconfigured to be a polyspecific CAR that is polyspecific for an antigen combination described herein. In some instances, a bispecific CAR may be reconfigured to be polyspecific for the antigens of an antigen combination as described herein. Useful bispecific CARs that may be reconfigured accordingly, include but are not limited to e.g., those bispecific CARs described in Grada et al., Mol Ther Nucleic Acids. (2013) 2:e105; Qin et al. Blood (2015) 126:4427; Liu et al. J Virol. (2015) 89(13):6685-94; Hegde et al. Journal for ImmunoTherapy of Cancer (2015) 3(Suppl 2):03; US Patent Application Pub. Nos. 20180311269, 20180230225, 20180079824, 20170107285, 20170073423, 20160303230, 20160207989, 20150038684, and 20130280220; the disclosures of which are incorporated herein by reference in their entirety.

In some instances, a PIIP of the present disclosure may be a polyspecific TCR and the immune response induced may be a TCR-specific immune response. TCR-specific immune responses include an immune response induced by the TCR binding its target(s) and signaling through an intracellular signaling domain of the TCR. The results of a TCR-specific immune response may vary and may include e.g., immune activation of an immune cell expressing the TCR, increased proliferation of an immune cell expressing the TCR, secretion and/or expression (including increased secretion and/or expression) of an immune molecule (e.g., a cytokine) by an immune cell expressing the TCR, increased cytotoxic activity of an immune cell expressing the TCR, increased killing of a cell to which the TCR is targeted, etc.

In some instances, a polyspecific TCR of the present disclosure may be derived from a previously investigated or otherwise available TCR, e.g., by replacing or amending the antigen-binding portion of the previously investigated or otherwise available TCR with one or more antigen binding domains such that the resulting polyspecific TCR is specific for the desired antigen combination, including e.g., an antigen combination described herein. Put another way, in some instances, an existing TCR may be reconfigured to be a polyspecific TCR that is polyspecific for an antigen combination described herein. In some instances, a bispecific TCR may be reconfigured to be polyspecific for the antigens of an antigen combination as described herein. Useful bispecific TCRs that may be reconfigured accordingly, include but are not limited to e.g., US Patent Application Pub. Nos. 20180311269, 20180258422, 20180201926, 20170268056, 20170015727, 20160355567, 20160244825, 20160213750, 20140242025, 20140205560, 20140134128, 20130040836, 20120177595, 20100233167, and 20100009863; the disclosures of which are incorporated herein by reference in their entirety.

In some instances, a PIIP of the present disclosure may be a polyspecific antibody and the immune response induced may be an antibody-specific immune response. Antibody-specific immune responses induced by a PIIP of the present disclosure will vary and may include e.g., antibody-dependent cellular cytotoxicity (ADCC) immune responses, complement-dependent cytotoxicity (CDC) immune responses, and the like. Antibody-specific immune responses include an immune response induced by the antibody binding its target(s) and, e.g., ADCC and/or CDC immune responses dependent thereon. The results of an antibody-specific immune response may vary and may include e.g., cell-mediated lysis of a target cell bound by the antibody, membrane complex-mediated lysis of a target cell bound by the antibody, and the like.

Immune modulation by polyspecific antibodies is not limited to modulation through ADCC and CDC pathways. In some instances, immunomodulation by an antibody can be Fc-dependent or Fc-independent and can include increased uptake of antigen via FcR on antigen-presenting cells, differential engagement of stimulatory versus inhibitory FcR, FcR-dependent enhancement of MHC class I-restricted cross-presentation, alterations in proteolysis and antigen processing, a shift in presentation of class II-restricted T-cell determinants, changes in cytokine expression by antigen-presenting cells and/or T cells, masking of dominant epitopes by the antibody, exposure of cryptic epitopes induced by antibody binding, enhanced germinal center formation and generation of strong recall responses, changes in usage of germline-encoded VH genes, induction of somatic hypermutation. and the like. See e.g., Brady L J Infect Immun (2005) 73(2): 671-678; the disclosure of which is incorporated herein by reference in its entirety.

In some instances, a polyspecific antibody of the present disclosure may be derived from a previously investigated or otherwise available antibody, e.g., by replacing or amending the antigen-binding portion of the previously investigated or otherwise available antibody with one or more antigen binding domains such that the resulting polyspecific antibody is specific for the desired antigen combination, including e.g., an antigen combination described herein. Put another way, in some instances, an existing antibody may be reconfigured to be a polyspecific antibody that is polyspecific for an antigen combination described herein.

Useful antibodies that may be reconfigured as a polyspecific antibody having polyspecificity for an antigen combination described herein include but are not limited therapeutic antibodies, such as e.g., 8H9, Abagovomab, Abciximab, Abituzumab, Abrilumab, Actoxumab, Aducanumab, Afelimomab, Afutuzumab, Alacizumab pegol, ALD518, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab/tocilizumab, Atorolimumab, Bapineuzumab, Basiliximab, Bavituximab, Bectumomab, Begelomab, Benralizumab, Bertilimumab, Besilesomab, Bevacizumab/Ranibizumab, Bezlotoxumab, Biciromab, Bimagrumab, Bimekizumab, Bivatuzumab mertansine, Blosozumab, Bococizumab, Brentuximabvedotin, Brodalumab, Brolucizumab, Brontictuzumab, Cantuzumab mertansine, Cantuzumab ravtansine, Caplacizumab, Capromab pendetide, Carlumab, Catumaxomab, cBR96-doxorubicin immunoconjugate, Cedelizumab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Dacetuzumab, Daclizumab, Dalotuzumab, Dapirolizumab pegol, Daratumumab, Dectrekumab, Demcizumab, Denintuzumab mafodotin, Derlotuximab biotin, Detumomab, Dinutuximab, Diridavumab, Dorlimomab aritox, Drozitumab, Duligotumab, Dupilumab, Durvalumab, Dusigitumab, Ecromeximab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Erlizumab, Ertumaxomab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gevokizumab, Girentuximab, Glembatumumab vedotin, Gomiliximab, Guselkumab, Ibalizumab, Ibalizumab, Icrucumab, Idarucizumab, Igovomab, IMAB362, Imalumab, Imciromab, Imgatuzumab, Inclacumab, Indatuximab ravtansine, Indusatumab vedotin, Inolimomab, Inotuzumab ozogamicin, Intetumumab, Iratumumab, Isatuximab, Itolizumab, Ixekizumab, Keliximab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Margetuximab, Maslimomab, Matuzumab, Mavrilimumab, Metelimumab, Milatuzumab, Minretumomab, Mirvetuximab soravtansine, Mitumomab, Mogamulizumab, Morolimumab, Morolimumab immune, Motavizumab, Moxetumomab pasudotox, Muromonab-CD3, Nacolomab tafenatox, Namilumab, Naptumomab estafenatox, Narnatumab, Nebacumab, Necitumumab, Nemolizumab, Nerelimomab, Nesvacumab, Nofetumomab merpentan, Obiltoxaximab, Obinutuzumab, Ocaratuzumab, Odulimomab, Olaratumab, Olokizumab, Onartuzumab, Ontuxizumab, Opicinumab, Oportuzumab monatox, Orticumab, Otlertuzumab, Oxelumab, Ozanezumab, Ozoralizumab, Pagibaximab, Palivizumab, Pankomab, Panobacumab, Parsatuzumab, Pascolizumab, Pasotuxizumab, Pateclizumab, Patritumab, Perakizumab, Pexelizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Rilotumumab, Rinucumab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teprotumumab, Tesidolumab, Tetulomab, TGN1412, Ticilimumab/tremelimumab, Tigatuzumab, Tildrakizumab, TNX-650, Toralizumab, Tosatoxumab, Tovetumab, Tralokinumab, TRBS07, Tregalizumab, Trevogrumab, Tucotuzumab celmoleukin, Tuvirumab, Ublituximab, Ulocuplumab, Urelumab, Urtoxazumab, Vandortuzumab vedotin, Vantictumab, Vanucizumab, Vapaliximab, Varlilumab, Vatelizumab, Veltuzumab, Vepalimomab, Vesencumab, Visilizumab, Vorsetuzumab mafodotin, Votumumab, Zalutumumab, Zanolimumab, Zatuximab, Ziralimumab, Zolimomab aritox, and the like.

Immune activation, through binding of a PIIP to one or more antigens for which the PIIP is polyspecific, may result in various outcomes. For example, in some instances, specific binding may increase production of a cytokine by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of cytokine produced in the absence of binding the antigen(s). Cytokines whose production can be increased include, but are not limited to, IL-2, interferon gamma (IFN-γ), tumor necrosis factor-alpha (TNF-α), IL-15, IL-12, IL-4, IL-5, IL-10, and the like.

In some instances, specific binding may increase secretion of a cytokine by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of cytokine secreted in the absence of binding the antigen(s).

In some instances, specific binding may increase expression of an immune activation marker by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of immune activation marker expression in the absence of binding the antigen(s). Useful immune activation markers include but are not limited to e.g., cytokines, CD69, NFAT, and the like.

In some instances, specific binding may increase cytotoxic activity by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity in the absence of binding the antigen(s).

In some instances, specific binding may reduce one or more of tumor growth rate, cancer cell number, and tumor mass, by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the tumor growth rate, cancer cell number, or tumor mass in the absence of binding the antigen(s).

Administration of a PIIPs of the present disclosure may vary and any convenient and appropriate method of administration may be employed. For example, in some instances, a PIIP may be directly administered to a subject in need thereof. For example, a PIIP, such as but not limited to a polyspecific antibody, may be directly administered to a subject, e.g., by injection, perfusion, or the like, in an appropriate pharmaceutical excipient. In some instances, a cell genetically modified to express a PIIP, e.g., a polyspecific CAR, a polyspecific TCR, a polyspecific antibody, etc., may be administered to a subject in need thereof. Accordingly, methods of the present disclosure include e.g., methods of killing a target cancer cell in an individual by administering to the individual an effective amount of a PIIP, including where the administering includes administering to the individual an effective amount of cytotoxic immune cells genetically modified to produce the PIIP. As such, the present disclosure also includes genetically modified cytotoxic immune cells, e.g., cells modified in vitro, ex vivo, or the like, where the cytotoxic immune cells are genetically modified to produce a PIIP as described herein.

### Genetically Modified Immune Cells

The present disclosure provides a cytotoxic immune cell genetically modified to produce two antigen-triggered polypeptides, each recognizing a different cell surface antigen.

To generate a genetically modified cytotoxic immune cell of the present disclosure, a parent cytotoxic immune cell is genetically modified to produce: a) a first antigen-triggered polypeptide that binds specifically to a first target cell surface antigen present on a target cancer cell; and b) a second antigen-triggered polypeptide that binds specifically to a second target cell surface antigen. Suitable parent cytotoxic immune cells include CD8⁺ T cells, natural killer (NK) cells, and the like. Thus, in some cases, a genetically modified cytotoxic immune cell of the present disclosure is a genetically modified CD8⁺ T cell. In other cases, a genetically modified cytotoxic immune cell of the present disclosure is a genetically modified NK cell.

In some cases, the target cancer cell is an acute myeloid leukemia cell, an anaplastic lymphoma cell, an astrocytoma cell, a B-cell cancer cell, a bone tumor cell, a breast cancer cell, a colon cancer cell, a gastric cancer cell, a glioblastoma cell, a glioma cell, a hepatocellular carcinoma cell, a leiomyosarcoma cell, a liposarcoma cell, a lung cancer cell, a mantle cell lymphoma cell, a melanoma cell, a neuroblastoma cell, a non small cell lung cancer cell, an oligodendroglioma cell, an ovarian cancer cell, a pancreatic cancer cell, a pancreatic ductal carcinoma cell, a prostate cancer cell, a renal cancer cell, a renal cell carcinoma cell, a sarcoma cell, a soft tissue sarcoma cell, a stomach cancer cell, or the like.

In some cases, a genetically modified cytotoxic immune cell of the present disclosure is genetically modified to express a first antigen-triggered polypeptide and a second antigen-triggered polypeptide that bind to antigens of a 2-input AND-gate target antigen pair. Non-limiting examples of 2-input AND gates (AND gates based on 2 target antigens) are depicted schematically in FIG. 7A.

For example, in some cases, the first antigen-triggered polypeptide is a BTTS and activation of the BTTS by binding to the first antigen (present on a target cancer cell) induces expression of the second antigen-triggered polypeptide. The second antigen-triggered polypeptide binds to the second antigen of the target antigen pair, where the second antigen is expressed on the surface of the target cancer cell. As an example, in some cases, the first antigen-triggered polypeptide is a BTTS and the second antigen-triggered polypeptide is a single chain CAR. As another example, in some cases, the first antigen-triggered polypeptide is a BTTS and the second antigen-triggered polypeptide is a TCR. For example, in some cases, the BTTS comprises an intracellular domain comprising a transcriptional activator, and activation of the BTTS by binding to the first antigen (present on a target cancer cell) induces release of the transcriptional activator; the released transcriptional activator activates transcription of the TCR or the single-chain CAR.

As another example, in some cases, the first antigen-triggered polypeptide is a BTTS and activation of the BTTS by binding to the first antigen (present on a target cancer cell) induces expression of the second antigen-triggered polypeptide, where the second antigen-triggered polypeptide is a heterodimeric (“two chain” or “split”) CAR comprising a first polypeptide chain and a second polypeptide chain. The heterodimeric CAR binds to the second antigen of the target antigen pair, where the second antigen is expressed on the surface of the target cancer cell. For example, in some cases, the first antigen-triggered polypeptide is a BTTS and the second antigen-triggered polypeptide is a split CAR (e.g., an ON-switch CAR). In some cases, activation of the BTTS by binding to the first antigen (present on a target cancer cell) induces expression of only the first polypeptide chain of the heterodimeric CAR; expression of the second polypeptide chain of the heterodimeric CAR can be constitutive. For example, in some cases, the BTTS comprises an intracellular domain comprising a transcriptional activator, and activation of the BTTS by binding to the first antigen (present on a target cancer cell) induces release of the transcriptional activator; the released transcriptional activator activates transcription of the first polypeptide chain of the heterodimeric CAR. In some cases, activation of the BTTS by binding to the first antigen (present on a target cancer cell) induces expression of only the second polypeptide chain of the heterodimeric CAR; expression of the first polypeptide chain of the heterodimeric CAR can be constitutive. Once the first polypeptide chain of the heterodimeric CAR is produced in the cell, it heterodimerizes with the second polypeptide chain of the heterodimeric CAR. As another example, in some cases, the BTTS comprises an intracellular domain comprising a transcriptional activator, and activation of the BTTS by binding to the first antigen (present on a target cancer cell) induces release of the transcriptional activator; the released transcriptional activator activates transcription of the second polypeptide chain of the heterodimeric CAR.

In some cases, a genetically modified cytotoxic immune cell of the present disclosure is genetically modified to express a first antigen-triggered polypeptide and a second antigen-triggered polypeptide that bind to antigens of a 2-input AND-NOT-gate target antigen pair. Non-limiting examples of 2-input AND-NOT gates (AND-NOT gates based on 2 target antigens) are depicted schematically in FIG. 7B.

As an example, in some cases, the first antigen-triggered polypeptide is a CAR, and the second antigen-triggered polypeptide is an iCAR. Binding of the iCAR to the second antigen (present on the surface of a non-cancerous cell, but not on the surface of a target cancer cell) of a target antigen pair inhibits T-cell activation mediated by activation of the CAR upon binding to the first antigen (present on the surface of the target cancer cell and on the surface of the non-cancerous cell) of the target antigen pair. As another example, in some cases, the first antigen-triggered polypeptide is a TCR, and the second antigen-triggered polypeptide is an iCAR. Binding of the iCAR to the second antigen (present on the surface of a non-cancerous cell, but not on the surface of a target cancer cell) of a target antigen pair blocks or reduces T-cell activation mediated by activation of the TCR upon binding to the first antigen (present on the surface of the target cancer cell and on the surface of the non-cancerous cell) of the target antigen pair.

As another example, in some cases, the first antigen-triggered polypeptide is a CAR, and the second antigen-triggered polypeptide is a BTTS comprising an intracellular domain that, when released upon activation of the BTTS by binding to the second target antigen, induces expression of an intracellular inhibitor that inhibits T-cell activation mediated by activation of the CAR upon binding to the first antigen (present on the surface of the target cancer cell and on the surface of the non-cancerous cell) of the target antigen pair. As another example, in some cases, the first antigen-triggered polypeptide is a TCR, and the second antigen-triggered polypeptide is a BTTS comprising an intracellular domain that, when released upon activation of the BTTS by binding to the second target antigen, induces expression of an intracellular inhibitor that inhibits T-cell activation mediated by activation of the TCR upon binding to the first antigen (present on the surface of the target cancer cell and on the surface of the non-cancerous cell) of the target antigen pair.

As another example, in some cases, the first antigen-triggered polypeptide is a CAR, and the second antigen-triggered polypeptide is a BTTS comprising an intracellular domain that, when released upon activation of the BTTS by binding to the second target antigen, induces expression of an extracellular inhibitor that inhibits T-cell activation mediated by activation of the CAR upon binding to the first antigen (present on the surface of the target cancer cell and on the surface of the non-cancerous cell) of the target antigen pair. As another example, in some cases, the first antigen-triggered polypeptide is a TCR, and the second antigen-triggered polypeptide is a BTTS comprising an intracellular domain that, when released upon activation of the BTTS by binding to the second target antigen, induces expression of an extracellular inhibitor that inhibits T-cell activation mediated by activation of the TCR upon binding to the first antigen (present on the surface of the target cancer cell and on the surface of the non-cancerous cell) of the target antigen pair.

### Systems for Inhibiting Cancer Cells

The present disclosure provides a system for inhibiting or killing a target cancer cell. A system of the present disclosure comprises: a) a first antigen-triggered polypeptide that binds specifically to a first target antigen present on the target cancer cell, or a first nucleic acid comprising a nucleotide sequence encoding the first antigen-triggered polypeptide; and b) a second antigen-triggered polypeptide that binds specifically to a second target antigen, or a second nucleic acid comprising a nucleotide sequence encoding the second antigen-triggered polypeptide.

In some cases, the target cancer cell is an acute myeloid leukemia cell, an anaplastic lymphoma cell, an astrocytoma cell, a B-cell cancer cell, a bone tumor cell, a breast cancer cell, a colon cancer cell, a gastric cancer cell, a glioblastoma cell, a glioma cell, a hepatocellular carcinoma cell, a leiomyosarcoma cell, a liposarcoma cell, a lung cancer cell, a mantle cell lymphoma cell, a melanoma cell, a neuroblastoma cell, a non small cell lung cancer cell, an oligodendroglioma cell, an ovarian cancer cell, a pancreatic cancer cell, a pancreatic ductal carcinoma cell, a prostate cancer cell, a renal cancer cell, a renal cell carcinoma cell, a sarcoma cell, a soft tissue sarcoma cell, a stomach cancer cell, or the like.

In some cases, as noted above, a system of the present disclosure comprises: a) a first antigen-triggered polypeptide that binds specifically to a first target antigen present on a target cancer cell; and b) a second antigen-triggered polypeptide that binds specifically to a second target antigen. In these instances, the polypeptides per se are introduced into an immune cell (e.g., CD8⁺ T cells and/or NK cells obtained from an individual). Methods of introducing polypeptides into a cell are known in the art; and any known method can be used. For example, in some cases, the first and the second antigen-triggered polypeptides comprise a protein transduction domain (PTD) at the N-terminus or the C-terminus of the polypeptides.

In some cases, as noted above, a system of the present disclosure comprises: a) a first nucleic acid comprising a nucleotide sequence encoding a first antigen-triggered polypeptide that binds specifically to a first target antigen present on a target cancer cell; and b) a second nucleic acid comprising a nucleotide sequence encoding a second antigen-triggered polypeptide that binds specifically to a second target antigen. In some cases, the first and the second antigen-triggered polypeptides are encoded by nucleotide sequences on separate nucleic acids. In other cases, the first and the second antigen-triggered polypeptides are encoded by nucleotide sequences present in the same nucleic acid. In some cases, the nucleic acid is a recombinant expression vector. In some cases, a system of the present disclosure comprises: a) a first recombinant expression vector comprising a nucleotide sequence encoding a first antigen-triggered polypeptide that binds specifically to a first target antigen present on a target cancer cell; and b) a second recombinant expression vector comprising a nucleotide sequence encoding a second antigen-triggered polypeptide that binds specifically to a second target antigen. In some cases, the nucleotide sequences are operably linked to a constitutive promoter. In some cases, the nucleotide sequences are operably linked to a regulatable promoter (e.g., an inducible promoter, a reversible promoter, etc.). In some cases, the nucleotide sequences are operably linked to an immune cell promoter, e.g., a T-cell specific promoter. In some cases, a system of the present disclosure comprises a recombinant expression vector comprising nucleotide sequences encoding: a) a first antigen-triggered polypeptide that binds specifically to a first target antigen present on a target cancer cell; and b) a second antigen-triggered polypeptide that binds specifically to a second target antigen. In some cases, the nucleotide sequences are operably linked to a constitutive promoter. In some cases, the nucleotide sequences are operably linked to a regulatable promoter (e.g., an inducible promoter, a reversible promoter, etc.). In some cases, the nucleotide sequences are operably linked to an immune cell promoter, e.g., a T-cell specific promoter.

Suitable promoters include, but are not limited to; cytomegalovirus immediate early promoter; herpes simplex virus thymidine kinase promoter; early and late SV40 promoters; promoter present in long terminal repeats from a retrovirus; a metallothionein-I promoter; and various art-known promoters. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some instances, nucleic acids present in a system of the present disclosure include immune cell specific promoters that are expressed in one or more immune cell types, including but not limited to lymphocytes, hematopoietic stem cells and/or progeny thereof (i.e., immune cell progenitors), etc. Any convenient and appropriate promoter of an immune cell specific gene may find use in nucleic acids of the present disclosure. In some instances, an immune cell specific promoter of a nucleic acid present in a system of the present disclosure may be a T cell specific promoter. In some instances, an immune cell specific promoter of a nucleic acid present in a system of the present disclosure may be a light and/or heavy chain immunoglobulin gene promoter and may or may not include one or more related enhancer elements.

In some instances, an immune cell specific promoter of a nucleic acid present in a system of the present disclosure may be a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9 γ (TRGV9) gene promoter, a V2 δ (TRDV2) gene promoter, and the like.

In some instances, an immune cell specific promoter present in a system of a nucleic acid of the present disclosure may be a viral promoter expressed in immune cells. As such, in some instances, viral promoters useful in nucleic acids present in a system of the present disclosure include viral promoters derived from immune cells viruses, including but not limited to, e.g., lentivirus promoters (e.g., HIV, SIV, FIV, EIAV, or Visna promoters) including e.g., LTR promoter, etc., Retroviridae promoters including, e.g., HTLV-I promoter, HTLV-II promoter, etc., and the like.

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90:7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

Expression vectors generally have convenient restriction sites located near the promoter sequence to provide for the insertion of nucleic acid sequences encoding heterologous proteins. A selectable marker operative in the expression host may be present. Suitable recombinant expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

### Antigen Combinations

A system of the present disclosure targets antigen combinations, where the targeting provides for specific killing of a target cancer cell. A system of the present disclosure targets antigen combinations, where the targeting provides for inducing a specific immune response to a target cancer cell. A genetically modified immune cell of the present disclosure targets antigen combinations, where the targeting provides for specific killing of a target cancer cell. A genetically modified immune cell of the present disclosure targets antigen combinations, where the targeting provides for inducing a specific immune response to a target cancer cell.

Antigen combinations may also reduce off-target effects and/or increase specificity for a target cancer cell, where e.g., an antigen combination includes one or more AND NOT combinations. Examples of target antigen combinations, and corresponding exemplary but non-limiting cancer that may be targeted, are listed in Table 1. Antigen combinations described herein are not limited to use in methods, cells and system having two different polypeptides and such combinations may also find use, in some instances, in polyspecific-immune inducing polypeptides. The following antigen combinations are exemplary, and not meant to be limiting.

Antigen combinations of interest are listed in Table 1.

### Methods of Killing Target Cancer Cells

The present disclosure provides methods for inducing an immune response to a target cancer cell and/or killing the target cancer cell. The present disclosure provides a method of inducing an immune response to a target cancer cell and/or killing a target cancer cell in an individual. In some cases, a method of the present disclosure for inducing an immune response to a target cancer cell and/or killing a target cell in an individual comprises: a) introducing a system of the present disclosure into an immune cell (e.g., a CD8⁺ T cell; an NK cell) obtained from the individual, generating a modified immune cell; and b) administering the modified immune cell to the individual, where the modified immune cell kills the target cancer cell in the individual. In some cases, the modified cytotoxic T cell does not substantially kill non-target cells such as non-cancerous cells.

In some cases, a method of the present disclosure for inducing an immune response to a target cancer cell and/or killing a target cell in an individual comprises administering to the individual an effective amount of a polyspecific-immune-inducing polypeptide (PIIP), where such administering may include e.g., delivering (e.g., through injection or other means) the PIIP to the subject, administering to the individual cytotoxic immune cells genetically modified to produce the PIIP, and the like.

The present disclosure provides a method of killing a target cancer cell in an individual. In some cases, a method of the present disclosure for killing a target cell in an individual comprises administering a genetically modified cytotoxic immune cell (e.g., a genetically modified CD8⁺ T cell; a genetically modified NK cell) of the present disclosure to the individual, where the genetically modified immune cell kills the target cancer cell in the individual. In some cases, the modified cytotoxic T cell does not substantially kill non-target cells such as non-cancerous cells.

Where the target antigen pair targeted by a method of the present disclosure is an AND-NOT gate target antigen pair, a method of the present disclosure provides for killing of a target cancer cell, but not a non-cancerous cell. For example, in some cases, a method of the present disclosure provides for a ratio of killing of cancer cells to non-cancerous cells of at least 10:1, at least 15:1, at least 20:1 at least 25:1, at least 50:1, at least 100:1, at least 500:1, at least 10³:1, at least 10⁴:1, or at least 10⁵:1.

Where the target antigen pair targeted by a method of the present disclosure is an AND gate target antigen pair, a method of the present disclosure provides for highly specific killing of a target cancer cell, and not a non-target (e.g., non-cancerous cell). For example, in some cases, a method of the present disclosure provides for a ratio of killing of cancer cells to non-cancerous cells of at least 10:1, at least 15:1, at least 20:1 at least 25:1, at least 50:1, at least 100:1, at least 500:1, at least 10³:1, at least 10⁴:1, or at least 10⁵:1.

### Methods Comprising Use of a System of the Present Disclosure

As noted above, in some cases, a method of the present disclosure for killing a target cell in an individual comprises: a) introducing a system of the present disclosure into an immune cell (e.g., a CD8⁺ T cell; an NK cell) obtained from the individual, generating a modified immune cell; and b) administering the modified immune cell to the individual, where the modified immune cell kills the target cancer cell in the individual. In some cases, the modified cytotoxic T cell does not substantially kill non-target cells such as non-cancerous cells.

T cells can be obtained from an individual (e.g., an individual having a cancer; an individual diagnosed as having a cancer; an individual being treated for a cancer with chemotherapy, radiation therapy, antibody therapy, surgery, etc.) using well-established methods. In some cases, a mixed population of cells is obtained from an individual; and CD8⁺ T cells and/or NK cells are isolated from the mixed population, such that a population of CD8⁺ T cells and/or NK cells is obtained that is at least 75%, at least 80%, at least 85%, at least 90%, at least 95%, or more than 98% pure, i.e., the purified cell population includes less than 25%, less than 20%, less than 15%, less than 10%, less than 5%, or less than 2%, of cells other than CD8⁺ T cells and or NK cells. A system of the present disclosure is then introduced into the purified CD8⁺ T cells and/or NK cells, to generate modified CD8⁺ T cells and/or modified NK cells that express the first antigen-triggered polypeptide and the second antigen-triggered polypeptide.

In some cases, as noted above, a system of the present disclosure comprises: a) a first antigen-triggered polypeptide that binds specifically to a first target antigen present on a target cancer cell; and b) a second antigen-triggered polypeptide that binds specifically to a second target antigen. In these instances, the polypeptides per se are introduced into an immune cell (e.g., CD8⁺ T cells and/or NK cells obtained from an individual). Methods of introducing polypeptides into a cell are known in the art; and any known method can be used. For example, in some cases, the first and the second antigen-triggered polypeptides comprise a protein transduction domain (PTD) at the N-terminus or the C-terminus of the polypeptides.

In some cases, as noted above, a system of the present disclosure comprises: a) a first nucleic acid comprising a nucleotide sequence encoding a first antigen-triggered polypeptide that binds specifically to a first target antigen present on a target cancer cell; and b) a second nucleic acid comprising a nucleotide sequence encoding a second antigen-triggered polypeptide that binds specifically to a second target antigen. In some cases, the first and the second antigen-triggered polypeptides are encoded by nucleotide sequences on separate nucleic acids. In other cases, the first and the second antigen-triggered polypeptides are encoded by nucleotide sequences present in the same nucleic acid. In some cases, the nucleic acid is a recombinant expression vector. In some cases, a system of the present disclosure comprises: a) a first recombinant expression vector comprising a nucleotide sequence encoding a first antigen-triggered polypeptide that binds specifically to a first target antigen present on a target cancer cell; and b) a second recombinant expression vector comprising a nucleotide sequence encoding a second antigen-triggered polypeptide that binds specifically to a second target antigen. In some cases, the nucleotide sequences are operably linked to a constitutive promoter. In some cases, the nucleotide sequences are operably linked to a regulatable promoter (e.g., an inducible promoter, a reversible promoter, etc.). In some cases, the nucleotide sequences are operably linked to an immune cell promoter, e.g., a T-cell specific promoter. In some cases, a system of the present disclosure comprises a recombinant expression vector comprising nucleotide sequences encoding: a) a first antigen-triggered polypeptide that binds specifically to a first target antigen present on a target cancer cell; and b) a second antigen-triggered polypeptide that binds specifically to a second target antigen. In some cases, the nucleotide sequences are operably linked to a constitutive promoter. In some cases, the nucleotide sequences are operably linked to a regulatable promoter (e.g., an inducible promoter, a reversible promoter, etc.). In some cases, the nucleotide sequences are operably linked to an immune cell promoter, e.g., a T-cell specific promoter. Suitable promoters include, but are not limited to; cytomegalovirus immediate early promoter; herpes simplex virus thymidine kinase promoter; early and late SV40 promoters; promoter present in long terminal repeats from a retrovirus; a metallothionein-I promoter; and various art-known promoters. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some instances, nucleic acids present in a system of the present disclosure include immune cell specific promoters that are expressed in one or more immune cell types, including but not limited to lymphocytes, hematopoietic stem cells and/or progeny thereof (i.e., immune cell progenitors), etc. Any convenient and appropriate promoter of an immune cell specific gene may find use in nucleic acids of the present disclosure. In some instances, an immune cell specific promoter of a nucleic acid present in a system of the present disclosure may be a T cell specific promoter. In some instances, an immune cell specific promoter of a nucleic acid present in a system of the present disclosure may be a light and/or heavy chain immunoglobulin gene promoter and may or may not include one or more related enhancer elements.

In some instances, an immune cell specific promoter of a nucleic acid present in a system of the present disclosure may be a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9 γ (TRGV9) gene promoter, a V2 δ (TRDV2) gene promoter, and the like.

In some instances, an immune cell specific promoter present in a system of a nucleic acid of the present disclosure may be a viral promoter expressed in immune cells. As such, in some instances, viral promoters useful in nucleic acids present in a system of the present disclosure include viral promoters derived from immune cells viruses, including but not limited to, e.g., lentivirus promoters (e.g., human immunodeficiency virus (HIV), SIV, FIV, EIAV, or Visna promoters) including e.g., long terminal repeat (LTR) promoter, etc., Retroviridae promoters including, e.g., HTLV-I promoter, HTLV-II promoter, etc., and the like.

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90:7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

Expression vectors generally have convenient restriction sites located near the promoter sequence to provide for the insertion of nucleic acid sequences encoding heterologous proteins. A selectable marker operative in the expression host may be present. Suitable recombinant expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

A method of the present disclosure for killing a target cell in an individual comprising: a) introducing a system of the present disclosure into an immune cell (e.g., a CD8⁺ T cell; an NK cell) obtained from the individual, generating a modified immune cell; and b) administering the modified immune cell to the individual, where the modified immune cell kills the target cancer cell in the individual, involves administering an effective amount of the modified immune cells to the individual.

In some cases, an effective amount (e.g., an effective number) of agent or modified immune cells is an amount that, when administered in one or more doses to an individual having a cancer, decreases the number of cancer cells in the individual by at least about 10%, at least about 15%, at least about 20%, at least about 25%, at least about 30%, at least about 40%, at least about 50%, at least about 75%, at least about 80%, at least about 90%, at least about 95%, or at least 98%, compared to the number of cancer cells in the individual before said administration.

In some cases, from about 10² to about 10⁹ modified immune cells are administered to an individual in a single dose. In some cases, a single dose of modified immune cells disclosure contains from 10² to about 10⁴, from about 10⁴ to about 10⁵, from about 10⁵ to about 10⁶, from about 10⁶ to about 10⁷, from about 10⁷ to about 10⁸, or from about 10⁸ to about 10⁹ modified immune cells. In some cases, a single dose of modified immune cells is administered. Multiple doses can also be administered, as needed and/or as determined by a medical professional. For example, 2, 3, 4, 5, 6, 7, 8, 9, 10, or more than 10, doses can be administered. If multiple doses are administered, the multiple doses can be administered at various frequencies, including, e.g., once per week, twice per month, once per month, once every 2 months, once every 3 months, once every 4 months, once every 6 months, or once per year.

In some cases, the target cancer cell is an acute myeloid leukemia cell, an anaplastic lymphoma cell, an astrocytoma cell, a B-cell cancer cell, a bone tumor cell, a breast cancer cell, a colon cancer cell, a gastric cancer cell, a glioblastoma cell, a glioma cell, a hepatocellular carcinoma cell, a leiomyosarcoma cell, a liposarcoma cell, a lung cancer cell, a mantle cell lymphoma cell, a melanoma cell, a neuroblastoma cell, a non small cell lung cancer cell, an oligodendroglioma cell, an ovarian cancer cell, a pancreatic cancer cell, a pancreatic ductal carcinoma cell, a prostate cancer cell, a renal cancer cell, a renal cell carcinoma cell, a sarcoma cell, a soft tissue sarcoma cell, a stomach cancer cell, or the like.

### Methods Comprising Use of a Genetically Modified Cytotoxic T Cell of the Present Disclosure

As noted above, in some cases, a method of the present disclosure for killing a target cell in an individual comprises administering a genetically modified cytotoxic immune cell (e.g., a genetically modified CD8⁺ T cell; a genetically modified NK cell) of the present disclosure to the individual, where the genetically modified immune cell kills the target cancer cell in the individual. In some cases, the modified cytotoxic T cell does not substantially kill non-target cells such as non-cancerous cells.

T cells can be obtained from an individual (e.g., an individual having a cancer; an individual diagnosed as having a cancer; an individual being treated for a cancer with chemotherapy, radiation therapy, antibody therapy, surgery, etc.) using well-established methods. In some cases, a mixed population of cells is obtained from an individual; and CD8⁺ T cells and/or NK cells are isolated from the mixed population, such that a population of CD8⁺ T cells and/or NK cells is obtained that is at least 75%, at least 80%, at least 85%, at least 90%, at least 95%, or more than 98% pure, i.e., the purified cell population includes less than 25%, less than 20%, less than 15%, less than 10%, less than 5%, or less than 2%, of cells other than CD8⁺ T cells and or NK cells. The purified CD8⁺ T cells and/or NK cells are then genetically modified to express the first antigen-triggered polypeptide and the second antigen-triggered polypeptide.

A method of the present disclosure for killing a target cell in an individual comprising administering a genetically modified cytotoxic immune cell (e.g., a genetically modified CD8⁺ T cell; a genetically modified NK cell) of the present disclosure to the individual involves administering an effective amount of a genetically modified cytotoxic immune cell of the present disclosure to the individual.

In some cases, an effective amount (e.g., an effective number) of genetically modified cytotoxic immune cells of the present disclosure is an amount that, when administered in one or more doses to an individual having a cancer, decreases the number of cancer cells in the individual by at least about 10%, at least about 15%, at least about 20%, at least about 25%, at least about 30%, at least about 40%, at least about 50%, at least about 75%, at least about 80%, at least about 90%, at least about 95%, or at least 98%, compared to the number of cancer cells in the individual before said administration.

In some cases, from about 10² to about 10⁹ genetically modified cytotoxic immune cells of the present disclosure are administered to an individual in a single dose. In some cases, a single dose of genetically modified cytotoxic immune cells of the present disclosure contains from 10² to about 10⁴, from about 10⁴ to about 10⁵, from about 10⁵ to about 10⁶, from about 10⁶ to about 10⁷, from about 10⁷ to about 10⁸, or from about 10⁸ to about 10⁹ genetically modified cytotoxic immune cells of the present disclosure. In some cases, a single dose of genetically modified cytotoxic immune cells of the present disclosure is administered. Multiple doses can also be administered, as needed and/or as determined by a medical professional. For example, 2, 3, 4, 5, 6, 7, 8, 9, 10, or more than 10, doses can be administered. If multiple doses are administered, the multiple doses can be administered at various frequencies, including, e.g., once per week, twice per month, once per month, once every 2 months, once every 3 months, once every 4 months, once every 6 months, or once per year.

In some cases, the target cancer cell is an acute myeloid leukemia cell, an anaplastic lymphoma cell, an astrocytoma cell, a B-cell cancer cell, a bone tumor cell, a breast cancer cell, a colon cancer cell, a gastric cancer cell, a glioblastoma cell, a glioma cell, a hepatocellular carcinoma cell, a leiomyosarcoma cell, a liposarcoma cell, a lung cancer cell, a mantle cell lymphoma cell, a melanoma cell, a neuroblastoma cell, a non small cell lung cancer cell, an oligodendroglioma cell, an ovarian cancer cell, a pancreatic cancer cell, a pancreatic ductal carcinoma cell, a prostate cancer cell, a renal cancer cell, a renal cell carcinoma cell, a sarcoma cell, a soft tissue sarcoma cell, a stomach cancer cell, or the like.

### Individuals Suitable for Treatment

Individuals suitable for treatment using a method of the present disclosure include an individual having a cancer; an individual diagnosed as having a cancer; an individual being treated for a cancer with chemotherapy, radiation therapy, antibody therapy, surgery, etc.); an individual who has been treated for a cancer (e.g., with one or more of chemotherapy, radiation therapy, antibody therapy, surgery, etc.), and who has failed to respond to the treatment; an individual who has been treated for a cancer (e.g., with one or more of chemotherapy, radiation therapy, antibody therapy, surgery, etc.), and who initially responded to the treatment but who subsequently relapsed, i.e., the cancer recurred.

Cancers that can be treated with a method of the present disclosure include an acute myeloid leukemia, an anaplastic lymphoma, an astrocytoma, a B-cell cancer, a bone tumor, a breast cancer, a colon cancer, a gastric cancer, a glioblastoma, a glioma, a hepatocellular carcinoma, a leiomyosarcoma, a liposarcoma, a lung cancer, a mantle cell lymphoma, a melanoma, a neuroblastoma, a non small cell lung cancer, an oligodendroglioma, an ovarian cancer, a pancreatic cancer, a pancreatic ductal carcinoma, a prostate cancer, a renal cancer, a renal cell carcinoma, a sarcoma, a soft tissue sarcoma, a stomach cancer, or the like.

In some cases, an individual to which a treatment of the present disclosure is administered is an individual expressing one or more antigens relevant to the subject treatment, including e.g., one or more (including 2 or more) target (i.e., cancer) antigens and/or one or more non-target (i.e., non-cancer or normal) antigens. Antigen expression may be determined by any convenient means. For example, in some instances, a subject may be evaluated for expression (or lack thereof) of one or more antigens relevant to the subject treatment, including one or more or all of the antigens of a particular antigen combination utilized in the treatment. Such evaluations (i.e., antigen expression testing) may be performed at any convenient time before, during or after a particular treatment regimen and using any convenient sample obtained from a subject (e.g., a tissue sample, a biopsy sample, etc.). Evaluations of antigen expression may be employed predictively (e.g., to predict the efficacy of an antigen combination based therapy), concurrently (e.g., to confirm the expression of antigens of an antigen combination during therapy), retrospectively (e.g., to analyze the expression of antigens of an antigen combination after therapy, e.g., to correlate expression of treatment outcomes, e.g., as part of a clinical trial utilizing an antigen combination described herein), or the like.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Methods

Defining the space of candidate antigens: Potential candidate antigens were defined as genes with known or predicted cell surface expression, restricting the search space to current clinical targets and genes coding for transmembrane proteins. More specifically, we assembled a set of 29 unique clinical antigens along with their indications that have shown promise in the literature or are targets in currently active CAR or TCR trials and mapped them to their corresponding genes. To assemble the list of transmembrane proteins a list of putative transmembrane genes was produced then filtered by localization to the plasma membrane as annotated in the COMPARTMENTS database (Binder et al., 2014) with high confidence (level 3 or higher), yielding a list of 2,358 genes. The COMPARTMENTS database uses a combination of manually curated literature, text mining, high-throughput screens, and sequence prediction methods to make subcellular location predictions.

Gene expression data processing: Gene level RSEM processed TPM counts were gathered for healthy human tissue samples from the Genotype Tissue Expression (GTEx) project version 7 and gene level RSEM processed tumor samples from The Cancer Genome Atlas (TCGA) firehose. All together there were 21,486 samples covering 34 tissues and 33 cancer types. To remove differences due to technical variation and thus combine these data from these two different sources we applied batch correction using a parametric empirical Bayes framework using the COMBAT function in the SVA R package (Johnson et al., 2007).

Intelligent subsampling and data partitioning: To increase the speed of clustering score calculations as well as partitioning data into training and test sets we used geometric sketching (Hie et al., 2019). Geometric sketching allows one subsample the space of samples maintaining the overall structure of the data by fitting a plaid covering and sampling points from within each region of the covering. In simulations across 8 different sketch sizes for 5 iterations across 100 gene pairs (10 fixed genes paired with 10 random genes) no loss of performance was observed when calculating Davies-Bouldin and Manhattan distance but substantial gains in runtime. Based on these simulations we chose to use a sketch size of 20% of all data for calculating clustering-based scores as well as the training data for classification and the remaining 80% of the data was held out for testing classification models.

Clustering-based scores: Clustering was evaluate using an adapted Davies-Bouldin (DB) method to measure the ratio of within cluster spread to cluster distance. The case where there are 2 clusters: a tumor cluster (given set of tumor samples) and a tissue cluster (all normal tissues samples) were considered. Lower DB scores are better as they indicate less within cluster distance (more tightly packed samples) and more distance between the cluster centers (more distance between tumor and normals). More formally, the following equations were used to calculate DB:

$$D B = \frac{{S}_{i} + {S}_{j}}{{M}_{i , j}}$$

which measures the ratio of scatter between the target tumor type (Sᵢ) and the cluster of normal tissues (Sⱼ) to the distance between the two clusters. Scatter for each cluster is calculated using:

$${S}_{i} = \frac{1}{{T}_{i}} {\sum }_{{j = 1}^{}}^{{{T}_{i}}^{}} \vert {X}_{j} - {A}_{i} \vert$$

where Tᵢ is the number of samples in a given cluster and Xⱼ is the location of a given sample and its distance from its cluster centroid (Aᵢ).

The distance between the clusters, Mᵢ, is calculated by subtracting the distance of the two cluster centers.

Where Aᵢ is the centroid of the cancer cluster, and Aⱼ is the centroid of the normal tissue cluster.

To give extra weight to the distance between clusters, the Manhattan distance (d) between the normal and the tumor clusters was calculated and used this in the final clustering score. To compute a more interpretable clustering-based score to use throughout our search, log DB and log distance values were rescaled across all gene pairs and tumor samples to be between 0 and 1, where 1 represents the best (smallest) DB score and the largest scaled distance. The minimum of these two scores is the final clustscore, thus a clustscore of 1 has the smallest DB and largest distance. Formally,

$${clustscore}_{t , {p}_{i , j}} = {min}_{i , j} ( \frac{log ( {DB}_{t , {p}_{i , j}} ) - {min}_{t , x} ( log ( {DB}_{t , x} ) )}{{max}_{t , x} ( log ( {DB}_{t , x} ) ) - {min}_{t , x} ( log ( {DB}_{t , x} ) )} , \frac{log ( {d}_{t , {p}_{i , j}} ) - {min}_{t , x} ( log ( {d}_{t , x} ) )}{{max}_{t , x} ( log ( {d}_{t , x} ) ) - {min}_{t , x} ( log ( {d}_{t , x} ) )} )$$

where t is a tumor type and p_{i,j} is a pair of genes made up of gene i and gene j and the min and max scores are calculated over all pairs.

Search space reduction for triples: To reduce the number of transmembrane and clinical antigens for triple antigen search the performance of single antigens was studied to create a smaller set of potential antigens per tumor type. The intuition being that each antigen must contribute at least a small amount of improvement to be a high scoring triple. To be included in the set of putative antigens per cancer, a single antigen was required to have a Davies-Bouldin score<=5 and a Manhattan distance>2. This filtering reduced the potential antigens to the following: Acute Myeloid Leukemia: 525, Adrenocortical Carcinoma: 169, Bladder Urothelial Carcinoma: 68, Brain Lower Grade Glioma: 361, Breast Invasive Carcinoma: 48, Cervical Squamous Cell Carcinoma and Endocervical Adenocarcinoma: 118, Cholangiocarcinoma: 69, Colon Adenocarcinoma: 131, Esophageal Carcinoma: 40, Glioblastoma Multiforme: 274, Head and Neck Squamous Cell Carcinoma: 102, Kidney Chromophobe: 140, Kidney Renal Clear Cell Carcinoma: 30, Kidney Renal Papillary Cell Carcinoma: 115, Liver Hepatocellular Carcinoma: 110, Lung Adenocarcinoma: 27, Lung Squamous Cell Carcinoma: 59, Lymphoid Neoplasm Diffuse Large B-cell Lymphoma: 416, Mesothelioma: 93, Ovarian Serous Cystadenocarcinoma: 102, Pancreatic Adenocarcinoma: 36, Pheochromocytoma and Paraganglioma: 233, Prostate Adenocarcinoma: 60, Rectum Adenocarcinoma: 118, Sarcoma: 64, Skin Cutaneous Melanoma: 194, Stomach Adenocarcinoma: 35, Testicular Germ Cell Tumors: 125, Thymoma: 205, Thyroid Carcinoma: 72, Uterine Carcinosarcoma: 90, Uterine Corpus Endometrial Carcinoma: 86, and Uveal Melanoma: 299. The clustering scores were then calculated as described in the above section.

Evaluation of top clustering-based scores: The top 10 antigen pairs per antigen class (C:C, C:N, and N:N) were chosen for each tumor based on their clustering scores for a total of ˜330 pairs per tumor type. Within the top 10 per class per tumor a particular gene in a pair was only allowed to appear a maximum of two times, preventing potential pairs from being dominated by a single gene with high separation. The analysis was further restricted to single antigens that are high, and pairs of antigens that have at least one antigen predicted to have high expression (high:high, high:low, and low:high) pairs.

To calculate the discriminatory ability of any particular antigen or antigen combination decision tree (DT) models were constructed on the 20% training partition using antigen expression as features and evaluated performance on the held out 80% of the data. More explicitly, for antigen pairs the rpart R package was used to construct two single feature decision trees with c=−1 and a max depth=1 forcing each tree to have a single split. These splits were then used to draw a classification boundary and calculated precision (the proportion of predicted positives that are correct), recall (the proportion of real positives that are predicted positive), and F₁ scores (the harmonic mean of precision and recall), as shown in the following:

$${F}_{1} = \frac{precision \cdot recall}{precision + recall}$$

Construct Design: All synNotch receptors used in this study were built using the mouse Notch1 (NM_008714) minimal regulatory region (Ile1427 to Arg 1752). The following binding domains were engineered into synNotch receptors: α-AXL scFv (Grada et al., 2013; Hegde et al., 2013) and WO2012175691A1 and the α-CDH6 scFv clone V10 (WO2016024195A1). synNotch receptors were designed to include either Gal4 DNA-binding domain (DBD) VP64 fusion proteins as a synthetic transcription factor. All synNotch receptors contain an N-terminal CD8α signal peptide for membrane targeting. Following the CD8vsignal peptide, Gal4 synNotch receptors contain a myc tag for easy and orthogonal surface detection with α-myc AF647 (Cell Signaling #2233) AF488 (R&D Systems #IC8529G), respectively.

All CARs used in this study were designed by fusing scFvs to the human CD8α chain hinge and transmembrane domains and the cytoplasmic regions of the human 4-1BB and CD3ζ (signaling proteins. The following binding domains were engineered into CARs: α-AXL scFv (WO2012175691A1) and the CD27 extracellular domain. All CARs included an N-terminal V5 tag for easy detection with α-V5 PE (Thermo Fisher #12-6796-42). All CARs contain an N-terminal CD8α signal peptide.

For experiments with T cells expressing a synNotch receptor the Gal4 system was utilized, and the receptors were cloned into a modified pHR′ SIN:CSW vector containing a constitutive PGK promoter. For these experiments, the pHR′ SIN:CSW vector was also modified to make the response element plasmids. Five copies of a Gal4 DNA binding domain target sequence were cloned 5′ to a minimal CMV promoter. Also included in the response element plasmids is a PGK promoter that constitutively drives mCherry or BFP expression to easily identify transduced T cells. For all synNotch response element vectors, the inducible transgene (e.x. CAR or TCR) was cloned via a BamHI site in the multiple cloning site 3′ to the Gal4 response elements. All constructs were cloned via InFusion Cloning (Takara Bio #638910).

Primary Human T Cell Isolation and Culture: Primary CD4+ and CD8⁺ T cells were isolated from anonymous donor blood after apheresis by negative selection (STEMCELL Technologies #15062 and 15023). T cells were cryopreserved in RPMI-1640 (Corning #10-040-CV) with 20% human AB serum (Valley Biomedical, #HP1022) and 5% DMSO (Sigma-Aldrich #472301). After thawing, T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

Lentiviral Transduction of Human T Cells and Target cells: Lenti-X 293T packaging cells (Clontech #11131D) were cultured in medium consisting of Dulbecco's Modified Eagle Medium (DMEM) (Gibco #10569-010), 10% fetal bovine serum (FBS) (University of California, San Francisco [UCSF] Cell Culture Facility), and gentamicin (UCSF Cell Culture Facility). Fresh packaging cells were thawed after cultured cells reached passage 30.

Pantropic VSV-G pseudotyped lentivirus was produced via transfection of Lenti-X 293T cells with a pHR′ SIN:CSW transgene expression vector and the viral packaging plasmids pCMVdR8.91 and pMD2.G using Fugene HD (Promega #E2312). Primary T cells were thawed the same day, and after 24 hr in culture, were stimulated with Dynabeads Human T-Activator CD3/CD28 (Thermo Scientific #11131D) at a 1:3 cell:bead ratio. At 48 hr, viral supernatant was harvested, and the primary T cells were exposed to the virus for 24 hr. At day 5 post T cell stimulation, Dynabeads were removed and the T cells expanded until day 12 when they were rested and could be used in assays. T cells were sorted for assays with a FACs ARIA II on day 5 or 6 post T cell stimulation.

Cancer and Target Cell Lines: The cancer cell lines used were Raji B cell Burkitt lymphoma cells (ATCC #CCL-86), and 769-P renal cell carcinoma cells (ATCC #CRL-1933). Rajis and 769-Ps were cultured in RPMI 1640 with L-glutamine (Corning #10-040-CV) supplemented with 10% FBS. The immortalized healthy tissue cell lines or primary human cells were Beas2B lung epithelial cells (ATCC #CRL-9609). Beas2B cells were cultured in BEBM (Lonza #CC3171) supplemented with the BEGM kit (Lonza #CC-3170).

Levels of various antigens were determined via flow cytometry after staining cells with the following antibodies: α-CD70 APC (Biolegend #355109), α-AXL APC (R&D systems #FAB154A), and α-CDH6 AF647 (R&D systems #FAB2715R).

Antibody Staining and Flow Cytometry Analysis: All antibody staining for flow cytometry was carried out in wells of round-bottom 96-well tissue culture plates. Cells were pelleted by centrifugation of plates for 4 min at 400×g. Supernatant was removed and cells were resuspended in 50 uL PBS containing the fluorescent antibody of interest. Cells stained 25 minutes at 4° C. in the dark. Stained cells were then washed two times with PBS and resuspended in fresh PBS supplemented with 1% FBS and EDTA for flow cytometry with a BD LSR II. All flow cytometry data analysis was performed in FlowJo software (TreeStar).

In Vitro Stimulation of synNotch T cells: For all in vitro synNotch T cell assays (including both reporter and killing assays), T cells were co-cultured with target cells at a 1:1 ratio, with anywhere from 1e4-1e5 each/well. The Countess II Cell Counter (ThermoFisher) was used to determine cell counts for all assay set up. T cells and target cells were mixed in 96-well tissue culture plates in 200 uL T cell media, and then plates were centrifuged for 1 min at 400×g to initiate interaction of the cells. For assays with Raji, round-bottom 96-well plates were used. For assays with all other target cells, flat-bottom 96-well plates were used. Cells were co-cultured for anywhere from 18 to 96 hours before analysis via flow cytometry with a BD LSR II.

Flow cytometry-based T Cell Cytotoxicity Assay: For all cytotoxicity assays, synNotch T cells, constitutive CAR or untransduced T cells were co-cultured with target cells at a 1:1 ratio as described above. After intended periods of incubation, samples were centrifuged for 4 min at 400×g, after first being transferred to a round-bottom 96-well plate if necessary. Supernatant was then removed and cells were resuspended in PBS supplemented with 1% FBS and EDTA for flow cytometry with a BD LSR II. Control samples containing only the target cells were used to set up flow cytometry gates for live target cells based on forward and side scatter patterns. For assays with all other target cells, target cells were gated on low CellTrace Far Red dye (Thermo Fisher #C34564) or low CD3 staining, as T cells used in these assays were either labeled with CellTrace Far Red or the samples were stained with α-CD3 APC/Cy7 (Biolegend #317342) to specifically label T cells. The level of target cell survival was determined by comparing the fraction of target cells alive in the culture compared to treatment with untransduced T cell controls. Three technical replicates are performed for each experiment.

### Results

Pipeline for Identifying Antigen Combinations that Improve Tumor Discrimination

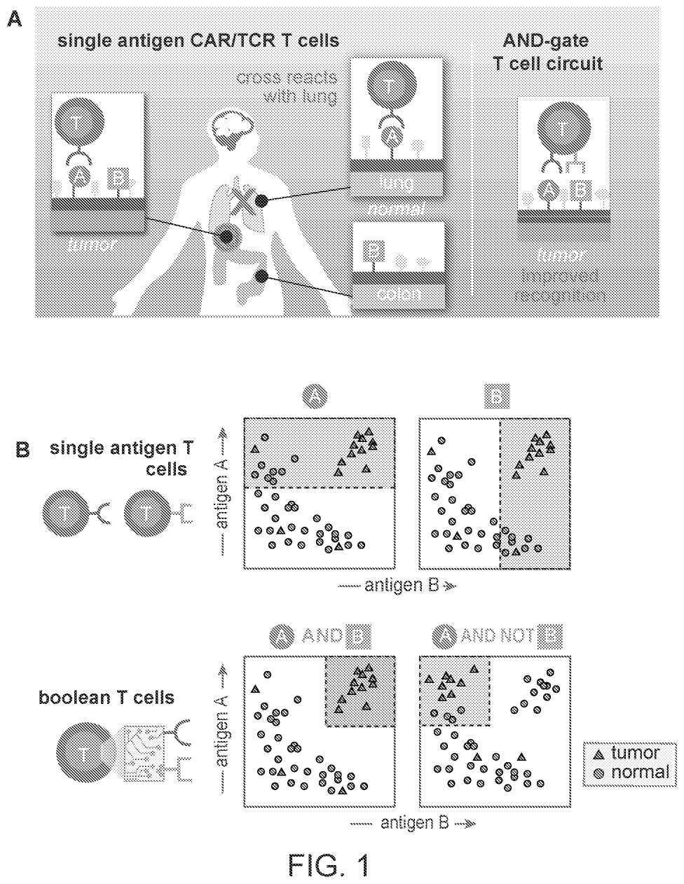

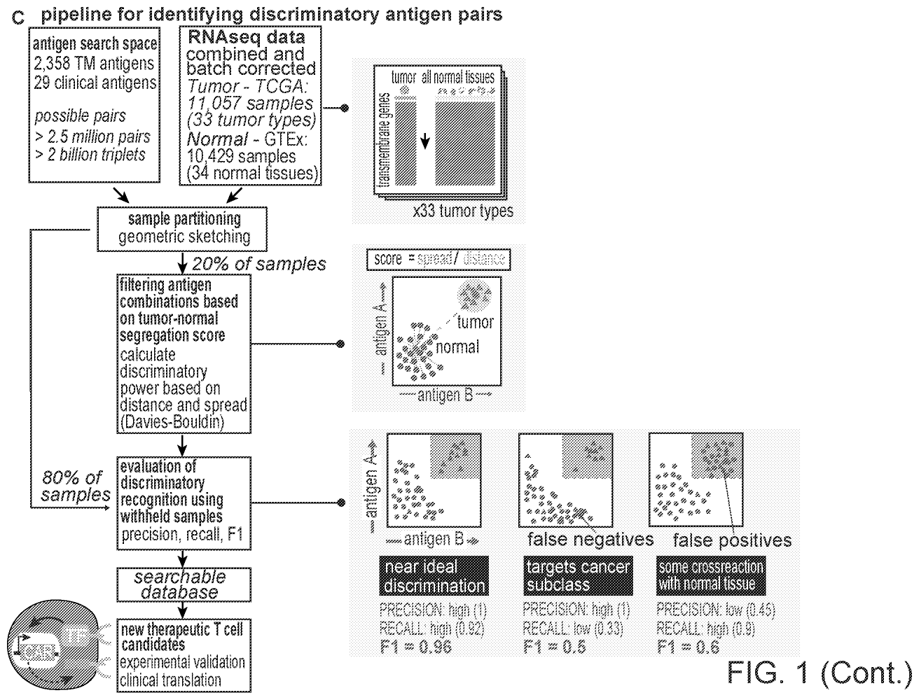

Candidate antigens must be recognizable from the cell surface. Towards that end a list of more than 5,000 genes expected to have cell surface expression was curated. Using the COMPARTMENTS database (Binder et al., 2014) we further pruned our curated list to only include predicted transmembrane proteins that are annotated to be expressed on the plasma membrane. Of these, the genes predicted to encode transmembrane proteins, potential target antigens were classified as either: “clinical”—involved as a target of a CAR T cell therapy in a currently registered clinical trial; or “novel”—not currently targeted in a known therapeutic T cell clinical trial. In total, this yielded approximately 2,400 surface expressed genes across 33 tumor types and 34 normal tissue samples (FIG. 1B).

RNAseq expression data across 9,084 samples taken from The Cancer Genome Atlas and 12,402 samples from Genotype Tissue Expression Project (GTEx) (GTEx Consortium, 2017) was used to measure the level of potential target antigen gene expression. To reduce expression differences due to technical variation we batch corrected all samples and used log transformed TPM (transcript per million) normalized read counts. Samples were partitioned using geometric sketching (Hie et al., 2019) to get an equal representation of all tissue types and the tumor samples in both partitions with 20% of the data taken for training and the remaining 80% set aside for evaluation.

Using the gene expression values of potential target antigens, a clustering-based score was calculated to quantify the separation between samples of a single tumor type versus all normal tissue samples (FIG. 1C). Specifically, the Davies-Bouldin metric was chosen, which measures the ratio of within cluster spread to between cluster distance, as the key component of the cluster-based scores. Before settling on Davies-Bouldin other cluster evaluation metrics were investigated that could be applied to the cluster separation problem including: Silhouette, Dunn's index, and the Xie-Bene validity measure. While all methods yield to similar results, some drawbacks with other metrics made Davies-Bouldin our preferred choice. Namely, that: Silhouette gives too much weight to compactness and did not have enough variation to differentiate between top antigens; Dunn's index did not produce enough variation in scores; and Xie-Bene generated too many missing values in practice.

Final clustering-based scores for a given antigen combination, utilize the Davies-Bouldin index with a modification to give extra weight for the distance between cluster centers. Together, clustering scores take into account the average distance between the two types of samples (tumor and normal) and the overall distribution of samples in expression space. Clustering-based scores are scaled from 0 to 1, for ease of ordering, with scores close to 1 indicating the best performing combinations with larger distance and less scatter between the classes of samples (see STAR Methods for additional details).

On the training set, clustering-based scores were used to rank all putative target antigens for each tumor type by their potential to separate samples of one tumor type from all normal tissue samples (FIG. 1C). Clustering-scores were calculated for all surface antigens as a single (n=2,358) and as a pair (n=2,778,903) for each tumor type. Only singles with high antigen expression in the target cancer samples, and pairs of antigens (doubles) that are either both highly expressed in the target (AND-gate), or one with high expression and the other with low expression in the target (AND-NOT-gate, insert number) are useful as viable CAR/TCR targets. Given the large number of surface antigens (2,358), the space of potential triplets for our set of antigens is >2.2 billion (2,358 choose 3), for efficiency we restricted the search of triple antigen gates to single antigens that have at least some discrimination potential as assessed in the single antigen search (see STAR Methods). Clustering-scores over this restricted set per tumor type for triple AND, AND-AND-NOT, and AND-NOT-NOT gates were then calculated.

The clustering-based scores prioritize antigens that have a large distance between tumor and normal samples, A metric that can more directly capture how much off-target toxicity can be avoided (precision) and the potential number of tumor samples we can target (recall) if Boolean logic gates are used was calculated. Decision tree classifiers can find boundaries that divide data into groups, while optimizing for the purity of the division. New samples can then be labeled with a group depending on which side of the boundary they fall on. In our case, decision trees can be used to find an expression value for each antigen where samples of a given tumor type are the most separated from normal tissue samples, then use the boundary to classify a new sample point as tumor or normal. Since clustering scores prioritize antigens that spread the two sample types, clear boundaries can be identified. To train the decision tree models the same training data as used for the clustering-based scores was used. The resulting models on our held out test set of samples was evaluated (data not shown). Applying this to the top antigen combinations found via clustering, an assessment of how well each of the top performing single, double, and triple gates separate tumor samples from normal tissue samples using the resulting F₁ score (harmonic mean of precision and recall) from classification was produced.

Recognition of all Cancer Types can be Improved by Adding Secondary Antigens to Current Clinical CAR T Targets

Clustering-based scores were calculated for the current clinically targeted antigens. These single antigen scores were compared with those obtained for antigen pairs in which two clinically targeted antigens (clinical antigens) are combined, a clinical antigen is paired with a novel putative surface antigen, or two novel surface antigens are paired. A spreadsheet listing the top 10 antigen pairs from each type of combination (e.g. clinical-clinical, clinical-novel, etc) per cancer type ranked by clustering-based scores was produced.

To provide more insight into how well antigens combinations separate normal vs tumor samples, decision tree models were used for each single antigen and each antigen pair in the top ranked antigens, as identified by their clustering scores. Decision trees find expression level cutoffs for each antigen that separate the classes of samples (tumor and normal). Applying them on held out sample data yielded additional metrics describing how well potential antigen combinations separate tumor and normal samples when using distinct expression level boundaries.

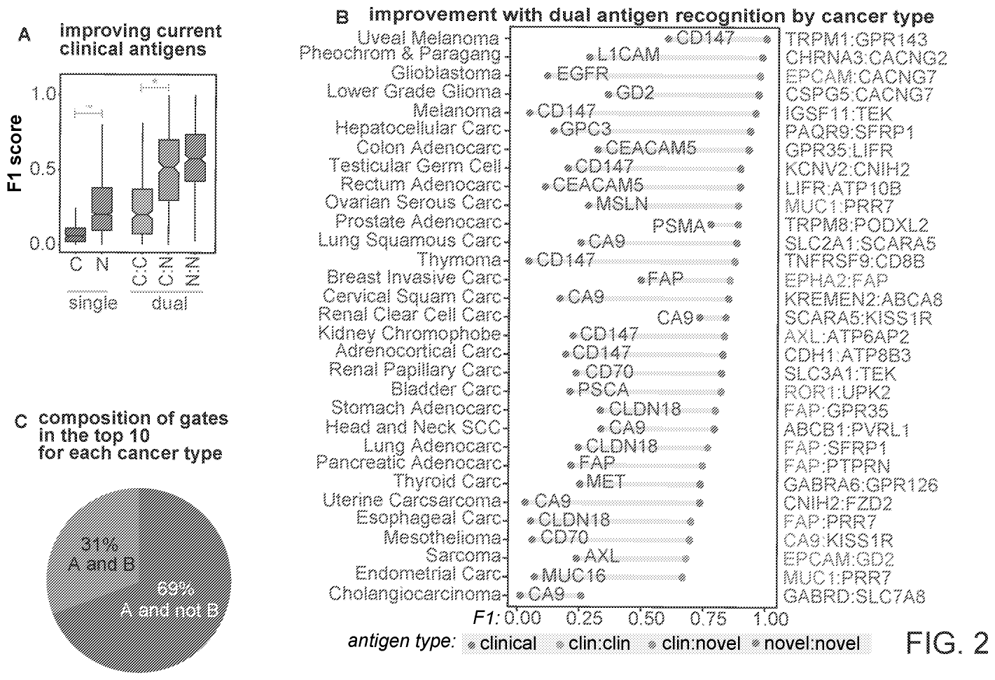

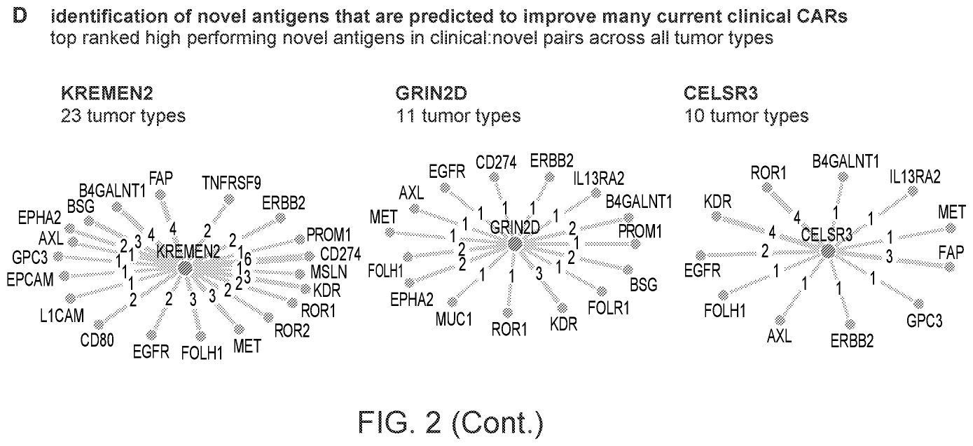

More specifically for each gate type, the top 10 antigen singles or pairs for each tumor (330 data points per gate type, from 33 tumors×10 top combos) were selected and quantified their tumor vs normal discrimination potential using F₁. As shown in FIG. 2A, current clinical antigens on average lack sensitivity and specificity when used as the sole recognition antigen (μF₁=0.09). However, combining two clinical antigens with AND or NOT logic for tumor recognition leads to significant improvement in both precision and recall as seen by the jump in F₁ (μₜₒₚ₁₀ F₁=0.25; Wilcoxon ranksum p=5.96×10⁻³²; n=669). This suggests that simple combinations of already well verified CAR targets can greatly improve the discriminatory ability of CAR T cells. Using the larger pool of novel antigens (that is, those identified by our pipeline that are not currently being investigated in clinical trials) allows for even more improvement in discrimination both alone, as single antigens (μF₁=0.37), and when paired with clinical antigens (μₜₒₚ₁₀ F₁=0.5; Wilcoxon ranksum p=1.33×10⁻⁴⁶; n=676). Novel-novel pairings show even more potential (μₜₒₚ₁₀ F₁=0.57). Taken together these results suggest that discrimination achievable by current clinical antigens can be dramatically improved by incorporating them into antigen pairs recognized by Boolean gated T cells.

The highest cluster-based scoring clinical and the highest antigen pair for each of the 33 individual cancer types were also examined (FIG. 2B). Thirty-one of the cancers examined showed marked improvement from the best clinical antigen to the best double antigen (μΔF₁=0.58). Among these best pairs per cancer type we saw reductions in overall cross-reactivity (μΔprec=0.76; n=31) and an increase in sensitivity (μΔrecall=0.12; n=31), with clinical-novel and novel-novel antigen pairs showing the best discrimination performance. Comparing the abundance of AND gates with AND-NOT gates reveals that AND-NOT gating is more common amongst the identified high performing antigen pairs (FIG. 2C).

Within the top 10 clinical-novel antigens for each of the 33 tumor types (330 pairs), a subset of novel antigens was identified that repeatedly form high-ranking antigen pairs with current clinical CAR targets across multiple cancers. The most frequently occurring three novel antigens are shown in in FIG. 2D, along with their highest-ranking clinical pairings and prevalence across tumor types. These novel antigens encode genes that have been noted by prior groups to be upregulated in individual tumors and play a role in tumorigenesis. This includes KREMEN2, a paralog of KREMEN1 that has recently been found to promote cell survival by blocking KREMEN1 homo-dimerization and induction of cell death (Sumia et al., 2019). GRIN2D a glutamate dependent NMDA receptor has previously been found to be up-regulated in cancer by IHC (Ferguson et al., 2016), confirming RNA based results of this work, and is believed to play a role tumor vascularization. The Cadherin EGF LAG seven-pass G-type receptor 3 (CELSR3), has also been identified to be upregulated in malignancies and is believed to play a role in cell cycle regulation (Xie et al., 2020). Taken together, these results highlight the power of the approach to systematically identify potentially useful novel antigens that can pair with current clinical antigens, across many different tumor types, which otherwise might have been “lost in the shuffle”.

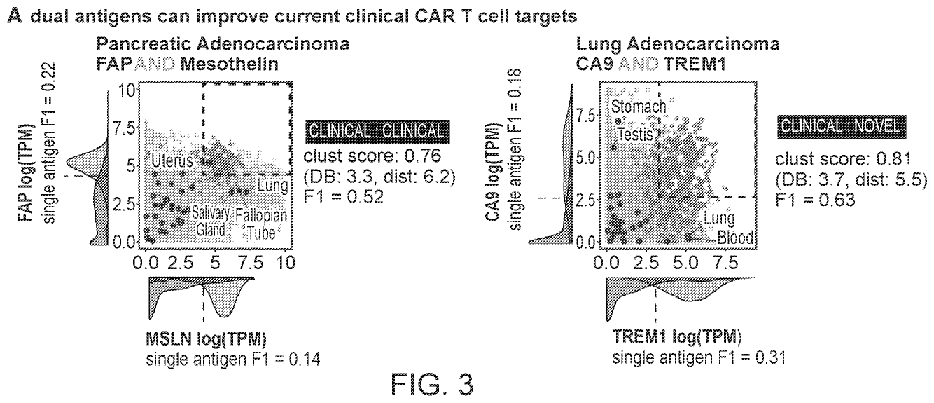

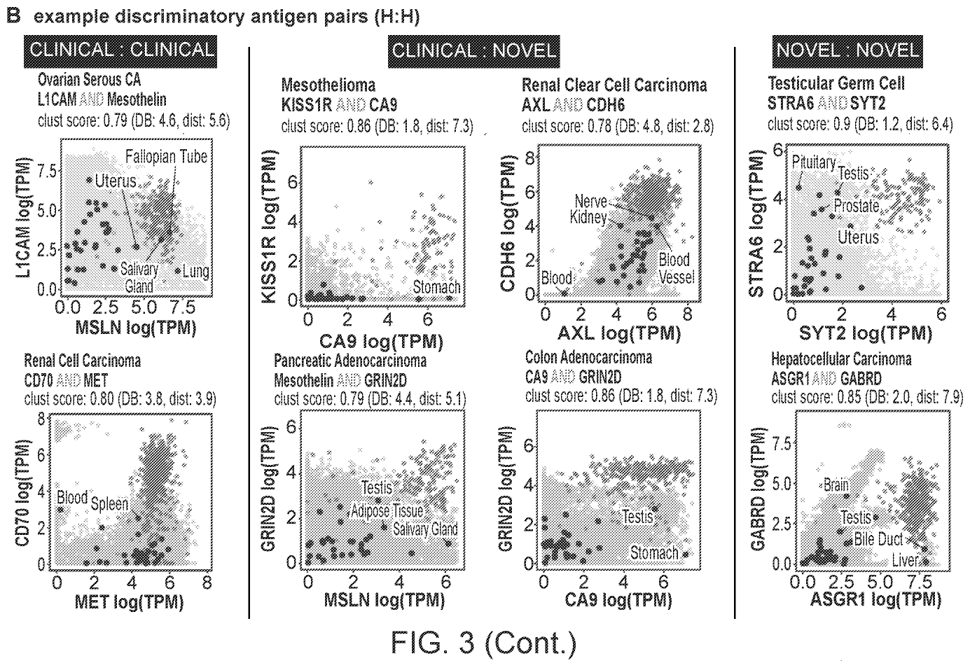

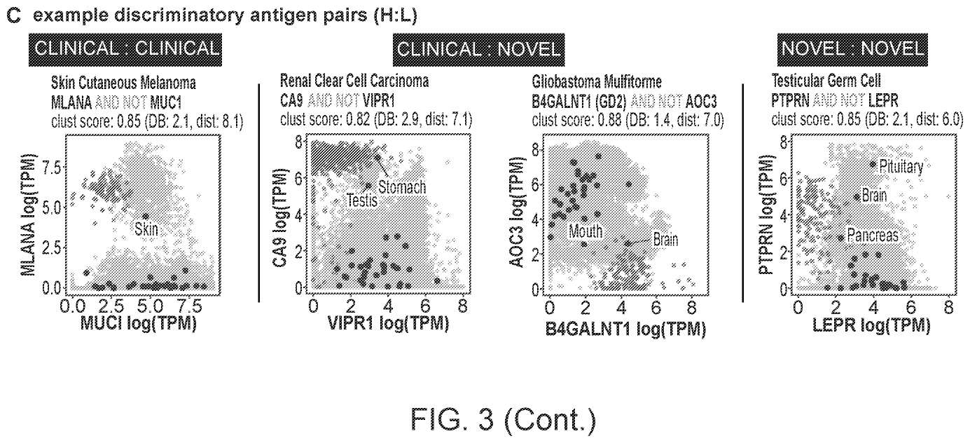

Examples of antigen pairs predicted to improve tumor recognition: Top possible antigen pairs as ranked by clustering score and their ability to discriminate a given cancer type were identified. In FIG. 3, a few examples of high-performing antigen pairs (high clustering scores) are highlighted. These 2D scatterplots show the RNAseq expression level (as log transformed TPM counts) of both antigens where each sample is represented by a point—red signifying cancer samples and light grey signifying normal tissue samples. Dark circles highlight the centroids for each normal tissue type, as labeled. In these plots, a high degree of separation occurs when a cluster of cancer (red) samples are segregated away from the bulk of normal tissue samples. This segregation can occur in the upper right quadrant (high:high representing AND gate); in the upper left quadrant (low:high representing AND-NOT gate), or lower right quadrant (high:low, AND-NOT gate).

The RNAseq expression data shows significant overlap between tumor and normal tissue for single clinical antigens currently being tested in as CAR targets in clinical trials (FIG. 3A), suggesting that true discrimination between tumor and normal tissue using single antigens may be quite difficult. This overlap is greatly reduced by combining information from both antigens, with a concomitant improvement in the calculated F₁ score. The plots shown in FIG. 3 represent only a small fraction of possible high performing combinations. Other examples were identified

Experimental Validation: Secondary Antigens that Improve CAR T Recognition of Renal Cell Carcinoma

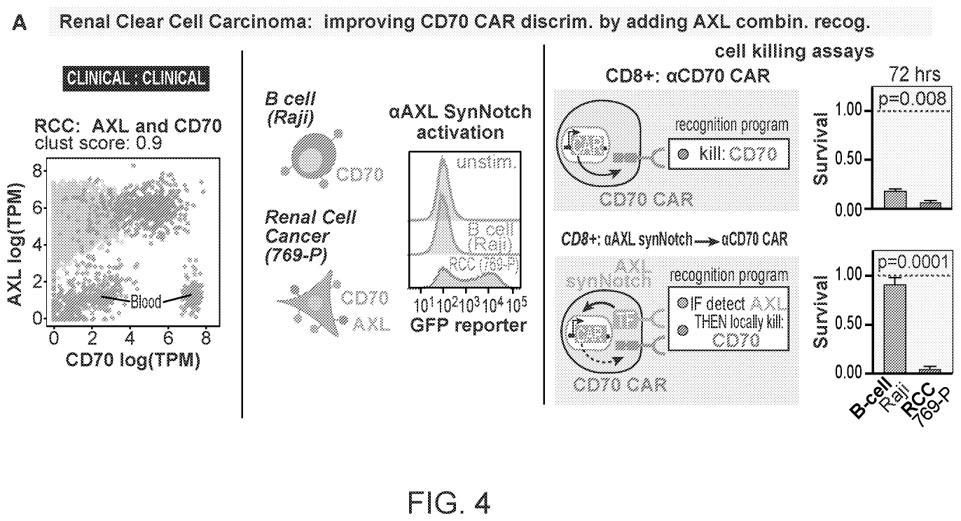

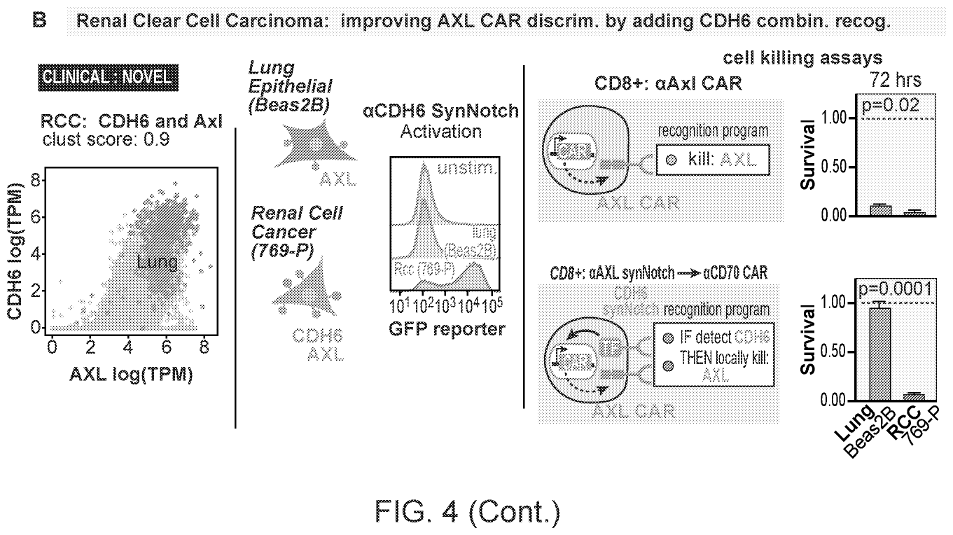

This analysis provides a very large dataset of potential antigen pairs (on the order of hundreds of thousands) for clinical translation as AND gate CAR T cells, many of which are actionable using currently available antigen recognition domains. To outline how antigen pairs can be translated to cellular design and to validate our bioinformatic predictions, a pair of engineered cell designs capable of specifically recognizing renal cell carcinoma (RCC) were constructed. Two predicted examples of combinatorial antigens for RCC recognition are shown in FIG. 4.

RCC is known to overexpress the tumor associated antigens CD70 and AXL (Jilaveanu et al., 2012; Yu et al., 2015) which we experimentally confirmed in an RCC cell line (769-P). Both of these antigens are currently involved in CAR T trials. However, as single CAR targets they are imperfect. CD70 is also expressed on a number of blood cells, including activated T cells, germinal center B cells, and dendritic cells in lymph nodes (Hintzen et al., 1994; Tesselaar et al., 2003). AXL is also expressed in many normal tissues including the lung (Qu et al., 2016). However, we find that the cross-reactive normal tissues for these two antigen targets are non-overlapping and, thus, the combination of these two complementary clinical antigen targets is predicted to greatly improve discrimination of tumor vs normal tissue (FIG. 4A).

To take advantage of this complementary pair of AND antigens for RCC, a CAR was engineered that recognizes CD70 using its cognate binding partner CD27 as the recognition domain (Wang et al., 2017). In vitro cytotoxicity assays showed that this CAR T cell was able to clear a renal cell carcinoma line (769-P) but also showed significant cytotoxicity against a B cell line (Raji cells). To create a T cell that recognizes AXL AND CD70, a synNotch receptor (Morsut et al., 2016) was used using an α-AXL scFv recognition domain fused to the Notch transmembrane domain and an orthogonal transcription factor (GAL4-VP64). It was found that T cells expressing an α-AXL synNotch that are co-cultured with RCC cells activate a synNotch GFP reporter; in contrast, the same T cells co-cultured with Raji B cells, which do not express AXL, do not activate the AXL synNotch receptor. AND gate T cells were engineered in which an α-AXL synNotch drives expression of a CD70 CAR. This AND circuit was found to cause the specific lysis of RCC cells, but not of Raji B cells (FIG. 4A). Thus, the combinatorial recognition of AXL AND CD70 improves upon the CD70 single target CAR, allowing discrimination between RCC cells and B cells.

Similarly, the single target α-AXL CAR is by itself a potential treatment for RCC (Zhu et al., 2019). However, as above targeting AXL is predicted to have toxic cross reactivity with lung tissue. An AXL CAR was constructed, and when expressed in human primary CD8⁺ T cells it was found to have cytotoxic activity against both an RCC cell line and an immortalized lung epithelial cell line (Beas2B) (FIG. 4B). Based on the current bioinformatics analysis of combinatorial antigens, it was predicted that the novel antigen CDH6 (cadherin 6), which has not previously been used as cellular therapy target, would improve the precision of an AXL CAR (FIG. 4B). CDH6 is a protein that mediates calcium-dependent cell-cell adhesion with PAX8 lineage linked expression (de Cristofaro et al., 2016) in the fetal kidney (Mbalaviele et al., 1998) as well as proximal tubule epithelium and is over-expressed in renal and ovarian cancer (Paul et al., 1997). A synNotch receptor targeting CDH6 was generated by screening four potential CDH6 scFv's fused to the synthetic notch core receptor. It was found that α-CDH6 synNotch receptors expressed in human primary T cells would specifically drive GFP reporter activity when co-cultured with an RCC cell line, but not with CDH6 negative lung epithelium cells (Beas2B). When an AND-gate T cell was constructed with α-CDH6 synNotch driving expression of an α-AXL CAR, it was found that specific lysis was only seen for the RCC cell line, and not the lung epithelial cell line. Thus, the combinatorial recognition of CDH6 and AXL improves upon the AXL single target CAR in that it allows discrimination between RCC cells and lung epithelial cells.

These two examples show that there are multiple ways to improve recognition of a specific cancer like RCC by harnessing combinatorial antigen recognition. In total, our analysis predicts 25 antigen pairs that discriminate RCC from normal tissues with a clustering score of >0.85. This set of experiments, illustrates a pipeline by which improved combinatorial CAR T circuits can be computationally identified and validated.

Triple Antigen Combinations Increase Precision of Cancer Recognition but with Tradeoff of Reduced Recall

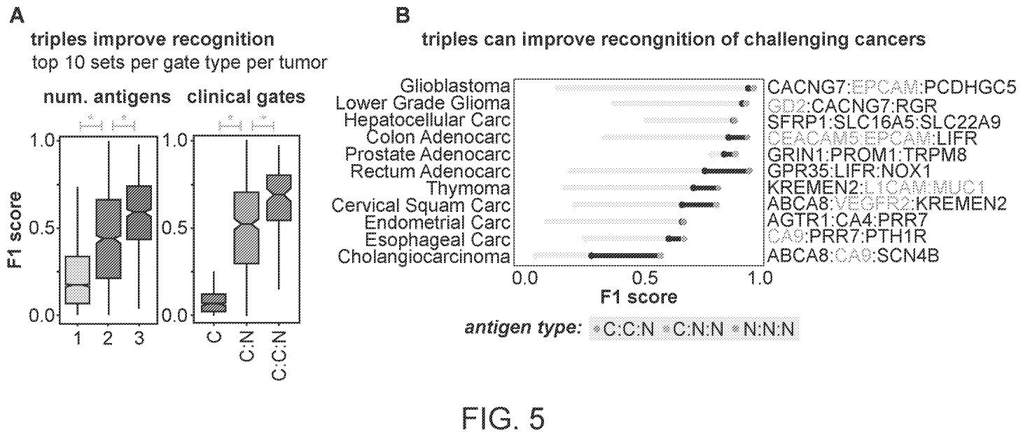

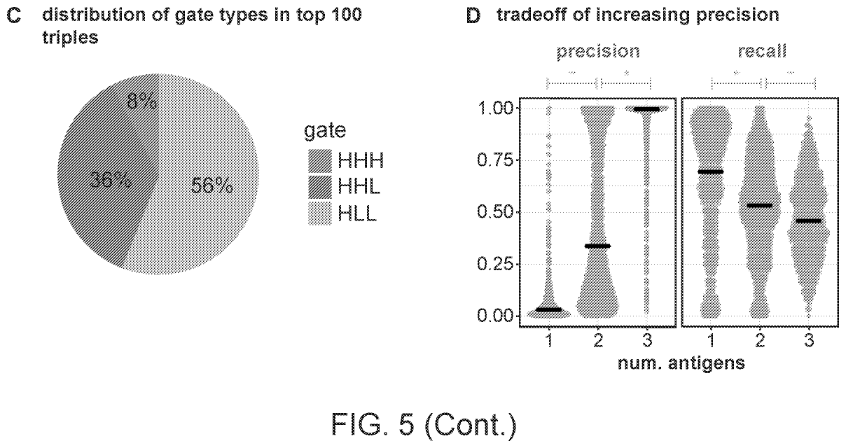

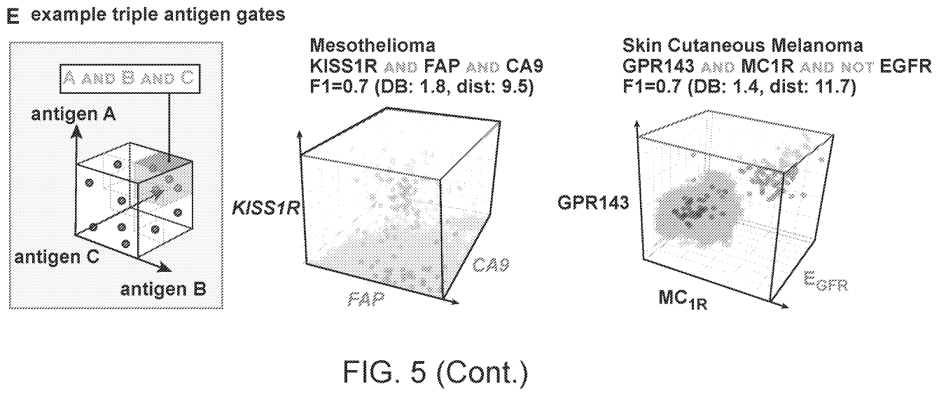

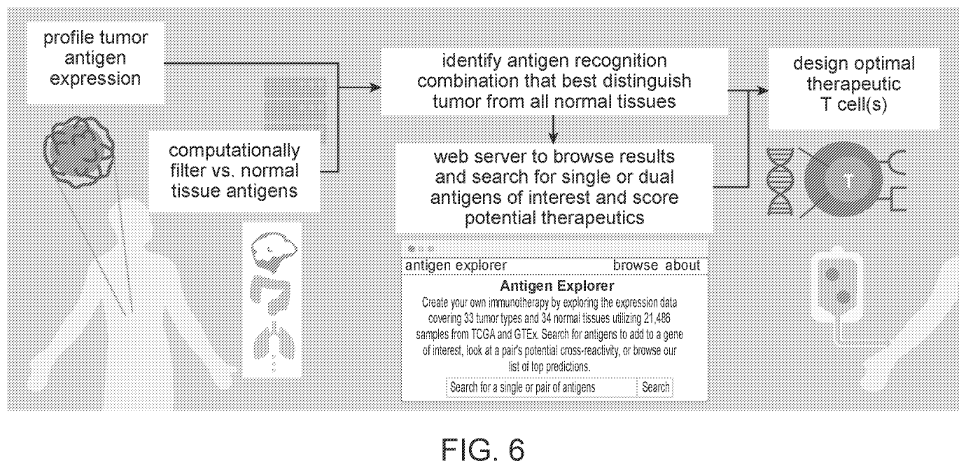

Adding a third antigen helps improve overall discrimination performance across tumor types. An overall increase in classification performance was observed with each additional antigen, averaged over the top 10 combos for all tumor and gate types (FIG. 5A). With significant increases in the mean F₁ from 1 to 2 antigens (μF₁ single=0.15; μₜₒₚ₁₀ F₁ double=0.37; Wilcoxon ranksum p=7.86×10⁻⁶⁸; n=2979) and for 2 to 3 antigens (μₜₒₚ₁₀ F₁ triple=0.58; Wilcoxon ranksum p=2.83×10⁴⁸; n=1578). Likewise, we see a significant increase in discrimination potential, moving from a clinical to a clinical-novel pair (μF₁ C=0.09; μₜₒₚ₁₀ F₁ C:N=0.5; Wilcoxon ranksum p=6.06×10⁻⁸¹; n=676) and to a clinical-clinical-novel triplet (μₜₒₚ₁₀ F₁ C:C:N=0.66, Wilcoxon ranksum p=5.00×10⁻⁸; n=438), suggesting that novels still have additional value when combined with two clinical antigens.

Looking across the top 100 gates per cancer it was found that the majority of triples (92%) have at least one NOT element, with over half (56%) having two antigens that have low expression in the target (AND-NOT-NOTs, FIG. 5C). Such a high percentage of NOTs further highlights the importance of synthetic NOT gates and our in silico approach, as more naïve approaches such as combining cancer specific markers in AND gates would miss many of the highest discriminatory combinations.

Perhaps the greatest benefit of adding a third antigen is the improvement that was observed in recognizing challenging cancers. Cholangiocarcinoma, in particular, was the tumor type that was the hardest to discriminate, with a max F₁ score of 0.26 for a pairing of two novel antigens. When adding a third antigen, predicted off-target toxicity was reduced and sensitivity was increased, by combining a lower scoring clinical novel pair with an additional novel antigen, increasing the max F₁ score by 0.31. Encouragingly, we also see substantial increases in the maximum discrimination performance for several other cancer types as well (FIG. 5B).

While in overall performance for many cancers was observed, for some tumor types a big gain in recognition going from two to three antigens was not observed. This is because overall the gains in precision come at a cost of reduced recall. Looking at both the precision and recall of the top combinations per each gate per tumor in FIG. 5D, it was seen that as one goes from one to two to three antigens nearly perfect average precision is achieved (p precision single=0.07, μₜₒₚ₁₀ precision double=0.44, μₜₒₚ₁₀ precision triple=0.90) and this increase is significant from one to two (Wilcoxon rank sum p=2.45×10⁻¹²⁰, n=2979) and two to three (Wilcoxon rank sum p=5.69×10⁻⁸⁴, n=1578). However, with each additional antigen there is a significant reduction in the average recall (μ recall single=0.66, μₜₒₚ₁₀ recall double=0.52, μₜₒₚ₁₀ recall triple=0.47; 1 to 2: Wilcoxon rank sum p=3.55×10⁻⁹, n=2979; 2 to 3: Wilcoxon rank sum p=5.94×10⁻⁴; n=1578). Taken together, we can conclude that 2-3 antigens are likely to be sufficient for precise recognition of most tumor types.

Like the examples shown in FIG. 3, a few examples of high-performing antigen triplets are highlighted in FIG. 5E. As in the 2D scatterplots, red dots are samples are those of a particular cancer type (e.g., mesothelioma (left) and melanoma (right)), light grey are normal tissues, and dark blue are highlight the normal tissue centroids. In both of these example triplets, the additional antigen gives a big boost to the overall separation of tumor and normal tissue samples, yielding fairly precise triplets that suffer slightly from reduced recall.

Doublet and triplet antigen combinations, their Boolean operators and the cancers to which the doublet and triplet antigen combinations can be applied as shown in Table 1 below. In this table, the score is the minimum of clustering-based score and an F₁ score, i.e., min(score, f1)). The combined score column estimates the discrimination potential of antigen combinations for a given indication, ranging from 1 (high discrimination) to 0 (no discrimination).

TABLE 1

| | | Combined |
|---|---|---|
| Antigen | Cancer | score |
| SPN AND NOT-ERBB2 | Acute Myeloid Leukemia | 0.931811676 |
| GPR52 AND NOT-EGFR | Acute Myeloid Leukemia | 0.930660486 |
| NOT-TNFRSF9 AND CACNG7 | Brain Lower Grade Glioma | 0.91752822 |
| NOT-ROR1 AND MLANA | Uveal Melanoma | 0.916684761 |
| NOT-ERBB2 AND CACNG7 | Glioblastoma Multiforme | 0.91524385 |
| BSG AND CACNG7 | Brain Lower Grade Glioma | 0.914058296 |
| NOT-KDR AND TAS2R13 | Acute Myeloid Leukemia | 0.912725436 |
| NOT-KDR AND OR52H1 | Acute Myeloid Leukemia | 0.912475499 |
| CHRNA3 AND NOT-ERBB2 | Pheochromocytoma and Paraganglioma | 0.909976118 |
| NOT-TNFRSF9 AND CSPG5 | Brain Lower Grade Glioma | 0.899847742 |
| ABCB5 AND NOT-ROR2 | Uveal Melanoma | 0.894345049 |
| CSPG5 AND B4GALNT1 | Brain Lower Grade Glioma | 0.89347079 |
| NOT-ROR1 AND CACNG2 | Pheochromocytoma and Paraganglioma | 0.892935696 |
| NOT-TNFRSF9 AND NTSR2 | Glioblastoma Multiforme | 0.883495146 |
| NOT-CD80 AND SLC1A3 | Brain Lower Grade Glioma | 0.882391008 |
| RGR AND B4GALNT1 | Brain Lower Grade Glioma | 0.874157303 |
| NOT-ROR1 AND PRLHR | Pheochromocytoma and Paraganglioma | 0.873873874 |
| PRR7 AND NOT-EGFR | Ovarian Serous Cystadenocarcinoma | 0.873010535 |
| KCNV2 AND B4GALNT1 | Testicular Germ Cell Tumors | 0.868199039 |
| NOT-LIFR AND CA9 | Rectum Adenocarcinoma | 0.854166667 |
| GPR19 AND B4GALNT1 | Glioblastoma Multiforme | 0.852459016 |
| NOT-MET AND NETO1 | Brain Lower Grade Glioma | 0.851764706 |
| FAP AND NOT-GHR | Lung Adenocarcinoma | 0.841974986 |
| NOT-ROR1 AND ULBP2 | Head and Neck Squamous Cell Carcinoma | 0.841949778 |
| NKAIN1 AND NOT-ERBB2 | Pheochromocytoma and Paraganglioma | 0.838709677 |
| LYPD1 AND BSG | Ovarian Serous Cystadenocarcinoma | 0.836565097 |
| NOT-ERBB2 AND NETO1 | Brain Lower Grade Glioma | 0.83518931 |
| NOT-ERBB2 AND KISS1R | Kidney Renal Clear Cell Carcinoma | 0.832425153 |
| NKAIN1 AND NOT-MUC1 | Pheochromocytoma and Paraganglioma | 0.824074074 |
| NOT-SCARA5 AND CA9 | Kidney Renal Clear Cell Carcinoma | 0.822709853 |
| NOT-MUC1 AND PRLHR | Pheochromocytoma and Paraganglioma | 0.820754717 |
| NOT-CD80 AND CACNG7 | Glioblastoma Multiforme | 0.816091954 |
| TRPM8 AND NOT-PROM1 | Prostate Adenocarcinoma | 0.811976048 |
| SLC1A3 AND NOT-ERBB2 | Glioblastoma Multiforme | 0.81025641 |
| LYPD1 AND BSG | Glioblastoma Multiforme | 0.804469274 |
| SLC2A1 AND NOT-ROR1 | Lung Squamous Cell Carcinoma | 0.802698145 |
| NOT-MUC1 AND SLC7A5 | Skin Cutaneous Melanoma | 0.8 |
| NOT-B4GALNT1 AND LAT | Thymoma | 0.794117647 |
| FAP AND NOT-MET | Breast Invasive Carcinoma | 0.79044396 |
| NOT-EPCAM AND B4GALNT1 | Glioblastoma Multiforme | 0.787878788 |
| NTSR2 AND BSG | Glioblastoma Multiforme | 0.784946237 |
| NOT-CD80 AND CNIH2 | Brain Lower Grade Glioma | 0.783847981 |
| NOT-FOLH1 AND KREMEN2 | Cervical Squamous Cell Carcinoma and | 0.783783784 |
| | Endocervical Adenocarcinoma | |
| FAP AND CELSR3 | Stomach Adenocarcinoma | 0.781954887 |
| NOT-ROR1 AND KREMEN2 | Head and Neck Squamous Cell Carcinoma | 0.778294574 |
| NOT-ERBB2 AND ATP8B3 | Adrenocortical Carcinoma | 0.777777778 |
| MLANA AND BSG | Uveal Melanoma | 0.776119403 |
| B4GALNT1 AND GRIN2D | Stomach Adenocarcinoma | 0.773399015 |
| NOT-ROR1 AND PAQR9 | Liver Hepatocellular Carcinoma | 0.773381295 |
| FAP AND KISS1R | Lung Adenocarcinoma | 0.768421053 |
| NOT-MET AND MSLN | Ovarian Serous Cystadenocarcinoma | 0.767195767 |
| CNIH2 AND NOT-ERBB2 | Brain Lower Grade Glioma | 0.76674938 |
| GABRD AND CD70 | Kidney Renal Clear Cell Carcinoma | 0.766081871 |
| ABCB5 AND NOT-FOLH1 | Uveal Melanoma | 0.764705882 |
| NOT-MET AND OR51E2 | Prostate Adenocarcinoma | 0.763197587 |
| GAPT AND NOT-ERBB2 | Acute Myeloid Leukemia | 0.761506276 |
| ULBP2 AND NOT-ERBB2 | Adrenocortical Carcinoma | 0.75862069 |
| NOT-KDR AND PRR7 | Ovarian Serous Cystadenocarcinoma | 0.757575758 |
| CXCR5 AND NOT-ERBB2 | Lymphoid Neoplasm Diffuse Large B-cell | 0.755555556 |
| | Lymphoma | |
| NOT-EPCAM AND BSG | Uveal Melanoma | 0.753623188 |
| NOT-EPHA2 AND OR2B6 | Breast Invasive Carcinoma | 0.747419133 |
| NOT-ROR1 AND GRIN2D | Colon Adenocarcinoma | 0.74351585 |
| OR2B6 AND NOT-MET | Breast Invasive Carcinoma | 0.740179187 |
| NOT-MUC1 AND TFR2 | Liver Hepatocellular Carcinoma | 0.735229759 |
| FAP AND KISS1R | Mesothelioma | 0.731707317 |
| GPR143 AND MLANA AND NOT- | Uveal Melanoma | 0.731210844 |
| ROR1 | | |
| NOT-FOLH1 AND LAT | Thymoma | 0.727272727 |
| TAS2R13 AND NOT-AXL | Acute Myeloid Leukemia | 0.725146199 |
| NOT-EPHA2 AND P2RX5 | Lymphoid Neoplasm Diffuse Large B-cell | 0.723404255 |
| | Lymphoma | |
| GPR52 AND NOT-AXL | Acute Myeloid Leukemia | 0.717647059 |
| NOT-GPR133 AND BSG | Kidney Chromophobe | 0.714285714 |
| NOT-KDR AND OR2B6 | Cervical Squamous Cell Carcinoma and | 0.713235294 |
| | Endocervical Adenocarcinoma | |
| FAP AND CELSR3 | Pancreatic Adenocarcinoma | 0.71 |
| NOT-ROR1 AND GPR35 | Colon Adenocarcinoma | 0.709812109 |
| NOT-ERBB2 AND NOT-MCAM | Acute Myeloid Leukemia | 0.706419501 |
| AND SPN | | |
| MET AND TSPAN33 | Kidney Renal Papillary Cell Carcinoma | 0.705882353 |
| NOT-EGFR AND MSLN | Ovarian Serous Cystadenocarcinoma | 0.704918033 |
| NOT-GPR133 AND CD70 | Kidney Renal Clear Cell Carcinoma | 0.703488372 |
| NOT-EGFR AND SLC7A5 | Skin Cutaneous Melanoma | 0.702702703 |
| SCARB1 AND NOT-L1CAM | Liver Hepatocellular Carcinoma | 0.701342282 |
| NOT-PROM1 AND CA9 | Head and Neck Squamous Cell Carcinoma | 0.700460829 |
| NOT-EGFR AND GPR52 AND | Acute Myeloid Leukemia | 0.699421775 |
| NOT-MCAM | | |
| NOT-EGFR AND GPR143 AND | Uveal Melanoma | 0.697777217 |
| MLANA | | |
| CD180 AND NOT-ERBB2 | Lymphoid Neoplasm Diffuse Large B-cell | 0.697674419 |
| | Lymphoma | |
| NOT-ERBB2 AND RHAG AND | Acute Myeloid Leukemia | 0.69469224 |
| SPN | | |
| MET AND NOT-LDLR | Kidney Renal Papillary Cell Carcinoma | 0.694444444 |
| NOT-EGFR AND GPR52 AND | Acute Myeloid Leukemia | 0.693316964 |
| RHAG | | |
| NOT-EPHA2 AND FAP | Lymphoid Neoplasm Diffuse Large B-cell | 0.692307692 |
| | Lymphoma | |
| ULBP2 AND B4GALNT1 | Head and Neck Squamous Cell Carcinoma | 0.691910499 |
| NOT-ROR1 AND SLC22A25 | Liver Hepatocellular Carcinoma | 0.69124424 |
| CALHM3 AND B4GALNT1 | Pancreatic Adenocarcinoma | 0.691099476 |
| SLC22A25 AND NOT-ERBB2 | Liver Hepatocellular Carcinoma | 0.688271605 |
| NOT-MUC1 AND BSG | Uveal Melanoma | 0.684931507 |
| NOT-ROR1 AND OR2B6 | Cervical Squamous Cell Carcinoma and | 0.68401487 |
| | Endocervical Adenocarcinoma | |
| NOT-FOLH1 AND GRIN2D | Colon Adenocarcinoma | 0.683615819 |
| NOT-ROR1 AND CEACAM5 | Colon Adenocarcinoma | 0.677290837 |
| NOT-EPCAM AND BSG | Brain Lower Grade Glioma | 0.673702726 |
| BSG AND NOT-LDLR | Kidney Renal Papillary Cell Carcinoma | 0.669950739 |
| FOLH1 AND NOT-SLC16A7 | Prostate Adenocarcinoma | 0.667752443 |
| NOT-EPHA2 AND ATP6AP2 | Kidney Chromophobe | 0.666666667 |
| NOT-ERBB2 AND CD70 | Lymphoid Neoplasm Diffuse Large B-cell | 0.666666667 |
| | Lymphoma | |
| B4GALNT1 AND NOT-ERBB2 | Pheochromocytoma and Paraganglioma | 0.666666667 |
| NOT-MET AND KCNK15 | Ovarian Serous Cystadenocarcinoma | 0.663341646 |
| CA9 AND NOX1 | Colon Adenocarcinoma | 0.6625 |
| GAPT AND NOT-MET | Acute Myeloid Leukemia | 0.661870504 |
| GPA33 AND NOT-KDR | Colon Adenocarcinoma | 0.661764706 |
| PRR7 AND NOT-FOLH1 | Stomach Adenocarcinoma | 0.657142857 |
| NOT-LIFR AND CA9 | Colon Adenocarcinoma | 0.653968254 |
| NOT-EGFR AND NOT-ERBB2 | Acute Myeloid Leukemia | 0.652000508 |
| AND SPN | | |
| NOT-EPCAM AND AXL | Brain Lower Grade Glioma | 0.648962656 |
| PSCA AND NOT-FOLR1 | Bladder Urothelial Carcinoma | 0.643835616 |
| NOT-EGFR AND NOT-MET AND | Acute Myeloid Leukemia | 0.643270074 |
| SPN | | |
| OR51E1 AND BSG | Prostate Adenocarcinoma | 0.64274571 |
| GPR35 AND NOT-FOLH1 | Colon Adenocarcinoma | 0.642706131 |
| ERBB2 AND NOT-GPC3 | Thyroid Carcinoma | 0.640990371 |
| NOX1 AND NOT-GPC3 | Rectum Adenocarcinoma | 0.640625 |
| NOT-ROR2 AND GPR19 | Skin Cutaneous Melanoma | 0.64 |
| NOT-AXL AND GAPT AND NOT- | Acute Myeloid Leukemia | 0.63803681 |
| PCDH1 | | |
| NOT-ROR1 AND SLC6A8 | Kidney Renal Clear Cell Carcinoma | 0.637911464 |
| NOT-RAMP3 AND CA9 | Ovarian Serous Cystadenocarcinoma | 0.63760218 |
| NOT-AXL AND NOT-PTPRF AND | Acute Myeloid Leukemia | 0.6375 |
| TAS2R46 | | |
| GPR19 AND NOT-ERBB2 | Testicular Germ Cell Tumors | 0.636363636 |
| NOT-CD274 AND ABCC4 | Prostate Adenocarcinoma | 0.631415241 |
| B4GALNT1 AND CACNG7 AND | Brain Lower Grade Glioma | 0.631234916 |
| RGR | | |
| NOT-ROR1 AND SLC6A17 AND | Uveal Melanoma | 0.631046482 |
| TRPM1 | | |
| NOT-MARVELD2 AND | Sarcoma | 0.630252101 |
| B4GALNT1 | | |
| ABCC11 AND NOT-GPC3 | Uveal Melanoma | 0.630136986 |
| NOT-KDR AND KREMEN2 | Uterine Carcinosarcoma | 0.62962963 |
| CD33 AND NOT-EPHA2 AND | Acute Myeloid Leukemia | 0.627564147 |
| GAPT | | |
| B4GALNT1 AND CACNG7 AND | Brain Lower Grade Glioma | 0.624549335 |
| NOT-SMAGP | | |
| CD33 AND NOT-EPHA2 AND | Acute Myeloid Leukemia | 0.622123593 |
| TAS2R30 | | |
| NOT-EGFR AND SLC6A17 AND | Uveal Melanoma | 0.619862585 |
| TRPM1 | | |
| CHRNA3 AND NOT-ERBB2 AND | Pheochromocytoma and Paraganglioma | 0.618256211 |
| NKAIN1 | | |
| NOT-AXL AND NOT-EPHA2 AND | Acute Myeloid Leukemia | 0.612897406 |
| GPR52 | | |
| NOT-ROR1 AND FZD2 | Uterine Corpus Endometrial Carcinoma | 0.612612613 |
| SLC6A17 AND BSG | Uveal Melanoma | 0.610169492 |
| CACNG2 AND NOT-ERBB2 AND | Pheochromocytoma and Paraganglioma | 0.609498522 |
| NKAIN1 | | |
| NOT-AXL AND GPR52 AND NOT- | Acute Myeloid Leukemia | 0.606354016 |
| KDR | | |
| EPHA2 AND GRIN2D | Esophageal Carcinoma | 0.606060606 |
| NOT-KDR AND BSG | Kidney Renal Papillary Cell Carcinoma | 0.60251046 |
| CD180 AND CXCR5 AND NOT- | Lymphoid Neoplasm Diffuse Large B-cell | 0.6 |
| ERBB2 | Lymphoma | |
| CD180 AND CD80 AND CXCR5 | Lymphoid Neoplasm Diffuse Large B-cell | 0.6 |
| | Lymphoma | |
| NOT-GPC3 AND ITGA4 AND | Acute Myeloid Leukemia | 0.6 |
| NOT-MTUS1 | | |
| EPCAM AND NOT-GPC3 | Colon Adenocarcinoma | 0.594488189 |
| NOT-MRGPRF AND BSG | Kidney Renal Papillary Cell Carcinoma | 0.594339623 |
| NOT-AXL AND CEACAM5 | Colon Adenocarcinoma | 0.593548387 |
| NOT-KDR AND SLC7A5 | Head and Neck Squamous Cell Carcinoma | 0.590984975 |
| SLC30A10 AND NOT-CD274 | Liver Hepatocellular Carcinoma | 0.589830508 |
| NOT-GPR126 AND ERBB2 | Thyroid Carcinoma | 0.58882402 |
| NOT-ERBB2 AND BSG | Adrenocortical Carcinoma | 0.588235294 |
| NOT-EPHA2 AND BSG | Kidney Chromophobe | 0.588235294 |
| NOT-CD274 AND PMEPA1 | Prostate Adenocarcinoma | 0.586092715 |
| B4GALNT1 AND CACNG7 AND | Glioblastoma Multiforme | 0.583880591 |
| NTSR2 | | |
| FAP AND ATP8B3 | Mesothelioma | 0.581818182 |
| CACNG7 AND NOT-EPCAM AND | Glioblastoma Multiforme | 0.58144704 |
| PCDHGC5 | | |
| CD80 AND KISS1R | Lung Adenocarcinoma | 0.581352834 |
| EPHB2 AND NOT-KDR | Colon Adenocarcinoma | 0.579124579 |
| NOT-MET AND KREMEN2 | Breast Invasive Carcinoma | 0.571755289 |
| FAP AND CALHM3 | Pancreatic Adenocarcinoma | 0.571428571 |
| NOT-KDR AND LY6K | Head and Neck Squamous Cell Carcinoma | 0.571069182 |
| NOT-EPHA2 AND LTA AND | Lymphoid Neoplasm Diffuse Large B-cell | 0.56841789 |
| PTPRCAP | Lymphoma | |
| CELSR3 AND NOT-FOLH1 | Lung Adenocarcinoma | 0.568062827 |
| NOT-EPHA2 AND KREMEN2 | Breast Invasive Carcinoma | 0.567887324 |
| ITGA4 AND NOT-KDR AND NOT- | Acute Myeloid Leukemia | 0.566371681 |
| TSPAN6 | | |
| MC1R AND NOT-ERBB2 | Skin Cutaneous Melanoma | 0.565217391 |
| NOT-ROR1 AND CHRNA5 | Bladder Urothelial Carcinoma | 0.56 |
| MLANA AND NOT-MUC1 AND | Uveal Melanoma | 0.559632922 |
| NOT-ROR1 | | |
| NOT-MET AND B4GALNT1 | Brain Lower Grade Glioma | 0.556311413 |
| BSG AND S1PR5 | Head and Neck Squamous Cell Carcinoma | 0.555885262 |
| SLC4A5 AND ERBB2 | Thyroid Carcinoma | 0.554782609 |
| NOT-EPHA2 AND ABCC4 | Prostate Adenocarcinoma | 0.551260504 |
| CHRNA3 AND NOT-MET AND | Pheochromocytoma and Paraganglioma | 0.549104737 |
| SLC4A8 | | |
| CD80 AND NOT-GHR | Lung Adenocarcinoma | 0.548063128 |
| NOT-GPC3 AND MLANA AND | Uveal Melanoma | 0.547033913 |
| NOT-ROR1 | | |
| NOT-KDR AND MET | Kidney Renal Papillary Cell Carcinoma | 0.546583851 |
| NOT-EGFR AND GPR143 AND | Uveal Melanoma | 0.546483947 |
| NOT-MUC1 | | |
| CSPG5 AND NOT-MET AND | Brain Lower Grade Glioma | 0.546048421 |
| NETO1 | | |
| B4GALNT1 AND CACNG7 AND | Brain Lower Grade Glioma | 0.545917909 |
| NOT-MET | | |
| B4GALNT1 AND NOT-ERBB2 | Brain Lower Grade Glioma | 0.544395924 |
| NOT-EGFR AND MUC16 | Ovarian Serous Cystadenocarcinoma | 0.544360902 |
| ITGA4 AND NOT-EGFR | Acute Myeloid Leukemia | 0.543307087 |
| CACNG2 AND NOT-MUC1 AND | Pheochromocytoma and Paraganglioma | 0.542313471 |
| PRLHR | | |
| NOT-ABCB1 AND B4GALNT1 | Lung Squamous Cell Carcinoma | 0.541176471 |
| PRR7 AND B4GALNT1 | Stomach Adenocarcinoma | 0.539823009 |
| FAP AND FZD2 | Uterine Carcinosarcoma | 0.53968254 |
| CSPG5 AND KCNA6 AND NOT- | Brain Lower Grade Glioma | 0.538986185 |
| MET | | |
| FAP AND NOT-F10 | Breast Invasive Carcinoma | 0.537931034 |
| CHRNA3 AND NOT-ERBB2 AND | Pheochromocytoma and Paraganglioma | 0.536810017 |
| NOT-MET | | |
| NOT-SLC4A4 AND BSG | Head and Neck Squamous Cell Carcinoma | 0.53515625 |
| NOT-KDR AND CHRNA5 | Bladder Urothelial Carcinoma | 0.533632287 |
| NOT-MUC1 AND BSG | Adrenocortical Carcinoma | 0.533333333 |
| GPR35 AND NOT-AXL | Rectum Adenocarcinoma | 0.531914894 |
| CACNG2 AND NOT-ERBB2 AND | Pheochromocytoma and Paraganglioma | 0.529894419 |
| NOT-MUC1 | | |
| B4GALNT1 AND GPR19 AND | Glioblastoma Multiforme | 0.529808504 |
| NTSR2 | | |
| NOT-ROR1 AND CA9 | Head and Neck Squamous Cell Carcinoma | 0.52917505 |
| CA9 AND NOT-FOLR1 | Bladder Urothelial Carcinoma | 0.527777778 |
| NOT-ROR1 AND CD72 | Lymphoid Neoplasm Diffuse Large B-cell | 0.526315789 |
| | Lymphoma | |
| BSG AND NOT-AXL | Kidney Chromophobe | 0.526315789 |
| NOT-EGFR AND LRMP AND | Lymphoid Neoplasm Diffuse Large B-cell | 0.526315789 |
| PTPRCAP | Lymphoma | |
| NOT-MET AND KREMEN2 | Prostate Adenocarcinoma | 0.521367521 |
| ABCC11 AND NOT-MUC1 AND | Uveal Melanoma | 0.519873377 |
| NOT-TNFSF10 | | |
| FAP AND NOT-MARVELD2 | Sarcoma | 0.518518519 |
| NOT-FOLH1 AND ATP8B3 | Mesothelioma | 0.518518519 |
| NOT-PROM1 AND PSCA | Bladder Urothelial Carcinoma | 0.518095238 |
| GPR35 AND CD80 | Stomach Adenocarcinoma | 0.517321016 |
| CELSR3 AND NOT-MUC1 AND | Pheochromocytoma and Paraganglioma | 0.517276904 |
| PRLHR | | |
| NOT-KDR AND FZD2 | Uterine Carcinosarcoma | 0.516129032 |
| NOT-EGFR AND NOT-GPC3 AND | Uveal Melanoma | 0.51598368 |
| GPR143 | | |
| B4GALNT1 AND NOT-TMEM88 | Breast Invasive Carcinoma | 0.51529052 |
| NOT-CD274 AND SLC22A9 | Liver Hepatocellular Carcinoma | 0.514204545 |
| NOT-MET AND KREMEN2 | Testicular Germ Cell Tumors | 0.513761468 |
| NOT-ABCB1 AND CA9 | Ovarian Serous Cystadenocarcinoma | 0.513157895 |
| CD79B AND LTA AND NOT- | Lymphoid Neoplasm Diffuse Large B-cell | 0.512820513 |
| ROR1 | Lymphoma | |
| NOT-EPCAM AND L1CAM | Skin Cutaneous Melanoma | 0.509803922 |
| NOT-ROR1 AND SLC4A5 | Thyroid Carcinoma | 0.50965251 |
| ABCB5 AND NOT-MUC1 AND | Uveal Melanoma | 0.508828247 |
| NOT-TNFSF10 | | |
| NOT-LIFR AND BSG | Colon Adenocarcinoma | 0.508695652 |
| NOT-ABCG2 AND CA9 | Mesothelioma | 0.508474576 |
| MUC16 AND NOT-GPC3 | Uterine Corpus Endometrial Carcinoma | 0.505747126 |
| ROR2 AND KISS1R | Mesothelioma | 0.5 |
| KREMEN2 AND NOT-MUC1 AND | Thymoma | 0.496647885 |
| NOT-SLC1A1 | | |
| KREMEN2 AND NOT-MUC1 AND | Thymoma | 0.496205996 |
| NOT-PCDHB4 | | |
| CNIH2 AND NOT-EPHA2 AND | Brain Lower Grade Glioma | 0.495592882 |
| RGR | | |
| ABCB5 AND ABCC11 AND NOT- | Uveal Melanoma | 0.495474778 |
| GPC3 | | |
| CD80 AND CA9 | Lung Adenocarcinoma | 0.494178525 |
| CNIH2 AND NOT-EPHA2 AND | Brain Lower Grade Glioma | 0.493998851 |
| NETO1 | | |
| GPR35 AND NOT-FOLH1 | Stomach Adenocarcinoma | 0.493197279 |
| NOT-AOC3 AND NOT-EPCAM | Glioblastoma Multiforme | 0.492419295 |
| AND PCDHGC5 | | |
| FAP AND NOT-KDR | Lung Squamous Cell Carcinoma | 0.491666667 |
| EPCAM AND NOT-KDR | Colon Adenocarcinoma | 0.488073394 |
| CELSR3 AND NOT-MET AND | Pheochromocytoma and Paraganglioma | 0.486824015 |
| NDRG4 | | |
| CD79B AND NOT-ERBB2 AND | Lymphoid Neoplasm Diffuse Large B-cell | 0.486486486 |
| P2RX5 | Lymphoma | |
| CD72 AND NOT-EGFR AND | Lymphoid Neoplasm Diffuse Large B-cell | 0.486486486 |
| LRMP | Lymphoma | |
| LYPD1 AND NOT-CD274 | Ovarian Serous Cystadenocarcinoma | 0.485294118 |
| CHRNA3 AND NOT-MET AND | Pheochromocytoma and Paraganglioma | 0.484165118 |
| NOT-MUC1 | | |
| CD79B AND NOT-GPC3 AND | Lymphoid Neoplasm Diffuse Large B-cell | 0.483266432 |
| NOT-ROR1 | Lymphoma | |
| NOT-ROR2 AND CEACAM5 | Lung Adenocarcinoma | 0.481973435 |
| NOT-EGFR AND MC1R AND | Pheochromocytoma and Paraganglioma | 0.481652454 |
| PTPRN | | |
| NOT-EGFR AND PTPRN AND | Pheochromocytoma and Paraganglioma | 0.481456462 |
| NOT-SLC15A2 | | |
| BAI2 AND NOT-EPCAM AND | Brain Lower Grade Glioma | 0.480171675 |
| SLC1A3 | | |
| NOT-EPCAM AND SLC1A3 AND | Brain Lower Grade Glioma | 0.479147981 |
| NOT-SMAGP | | |
| NOT-ROR1 AND LRMP | Lymphoid Neoplasm Diffuse Large B-cell | 0.47826087 |
| | Lymphoma | |
| B4GALNT1 AND CACNG7 AND | Glioblastoma Multiforme | 0.475492182 |
| NOT-FOLR1 | | |
| LY6G6D AND BSG | Rectum Adenocarcinoma | 0.473684211 |
| MET AND NOT-GPC3 | Kidney Renal Clear Cell Carcinoma | 0.471183013 |
| CA9 AND GPR35 AND NOT-LIFR | Colon Adenocarcinoma | 0.470886605 |
| NOT-PROM1 AND CA9 | Bladder Urothelial Carcinoma | 0.470833333 |
| MET AND NOT-PSCA | Thyroid Carcinoma | 0.470111449 |
| NOT-EPHA2 AND P2RX5 AND | Lymphoid Neoplasm Diffuse Large B-cell | 0.470049421 |
| NOT-PROM1 | Lymphoma | |
| EPCAM AND NOT-PSCA | Thyroid Carcinoma | 0.469221835 |
| NOT-ROR1 AND FAP | Lung Squamous Cell Carcinoma | 0.46875 |
| NOT-ROR1 AND FAP | Bladder Urothelial Carcinoma | 0.468208092 |
| MUC1 AND NOT-GPC3 | Breast Invasive Carcinoma | 0.467757459 |
| CD79B AND NOT-EPHA2 AND | Lymphoid Neoplasm Diffuse Large B-cell | 0.467376098 |
| NOT-PROM1 | Lymphoma | |
| CA9 AND GRIN2D AND NOT- | Colon Adenocarcinoma | 0.465656692 |
| LIFR | | |
| NOT-DSC2 AND BSG | Adrenocortical Carcinoma | 0.465116279 |
| NOT-KDR AND TSPAN33 | Kidney Renal Papillary Cell Carcinoma | 0.464480874 |
| B4GALNT1 AND NDRG4 AND | Pheochromocytoma and Paraganglioma | 0.463051758 |
| SLC10A4 | | |
| ST3GAL5 AND NOT-PROM1 | Thyroid Carcinoma | 0.46179966 |
| KREMEN2 AND BSG | Lung Squamous Cell Carcinoma | 0.461684011 |
| B4GALNT1 AND EPHA8 AND | Pheochromocytoma and Paraganglioma | 0.460570704 |
| GPR19 | | |
| NOT-EGFR AND GPR143 AND | Skin Cutaneous Melanoma | 0.460547743 |
| MC1R | | |
| NOT-MUC1 AND ROR2 | Thymoma | 0.459770115 |
| FAP AND NOT-ABCB1 | Lung Squamous Cell Carcinoma | 0.458100559 |
| GPA33 AND BSG | Colon Adenocarcinoma | 0.457350272 |
| GABRD AND NOT-GPC3 | Thyroid Carcinoma | 0.456996149 |
| CA9 AND KREMEN2 AND PRR7 | Ovarian Serous Cystadenocarcinoma | 0.456565088 |
| CSPG5 AND NOT-EPCAM AND | Brain Lower Grade Glioma | 0.453619222 |
| NOT-MET | | |
| CELSR3 AND NOT-MET | Pheochromocytoma and Paraganglioma | 0.453159041 |
| NOT-EPCAM AND NOT-LEPR | Uveal Melanoma | 0.452541967 |
| AND SLC7A5 | | |
| GABRD AND NOT-ERBB2 | Kidney Renal Clear Cell Carcinoma | 0.451910408 |
| B4GALNT1 AND CACNG2 AND | Pheochromocytoma and Paraganglioma | 0.451549496 |
| NOT-EGFR | | |
| NOT-AOC3 AND BSG | Ovarian Serous Cystadenocarcinoma | 0.451388889 |
| BAI2 AND HCN2 AND NOT- | Brain Lower Grade Glioma | 0.45137667 |
| MUC1 | | |
| NOT-ERBB2 AND HCN2 AND | Adrenocortical Carcinoma | 0.450892108 |
| NOT-VIPR1 | | |
| NOT-EGFR AND GPR143 AND | Skin Cutaneous Melanoma | 0.449863373 |
| GPR19 | | |
| NOT-EPCAM AND ADAM12 | Sarcoma | 0.449541284 |
| CA9 AND GPR35 AND NOT-LIFR | Rectum Adenocarcinoma | 0.449328601 |
| NOT-GPC3 AND GPR35 AND | Rectum Adenocarcinoma | 0.44914487 |
| NOX1 | | |
| NOT-GPC3 AND GRIN2D AND | Rectum Adenocarcinoma | 0.448769928 |
| NOX1 | | |
| GPR37L1 AND KCNA6 AND NOT- | Brain Lower Grade Glioma | 0.445013729 |
| MUC1 | | |
| GPR19 AND LYPD1 AND NOT- | Glioblastoma Multiforme | 0.444548023 |
| MET | | |
| CD37 AND CD70 AND CD83 | Lymphoid Neoplasm Diffuse Large B-cell | 0.444444444 |
| | Lymphoma | |
| NOT-AOC3 AND CNIH2 AND | Glioblastoma Multiforme | 0.44389693 |
| NOT-FOLR1 | | |
| NOT-LIFR AND MC1R AND NOT- | Skin Cutaneous Melanoma | 0.443379764 |
| MUC1 | | |
| NOT-EPCAM AND NOT-SGMS2 | Uveal Melanoma | 0.443259194 |
| AND SLC7A5 | | |
| CNIH2 AND NOT-FOLR1 AND | Glioblastoma Multiforme | 0.442649901 |
| SLC1A3 | | |
| CLDN18 AND NOT-FOLH1 | Stomach Adenocarcinoma | 0.440310078 |
| PRR7 AND NOT-EGFR | Uterine Carcinosarcoma | 0.44 |
| NOT-LIFR AND BSG | Skin Cutaneous Melanoma | 0.43902439 |
| CA9 AND GRIN2D AND NOT- | Rectum Adenocarcinoma | 0.436395011 |
| LIFR | | |
| B4GALNT1 AND NOT-EGFR AND | Pheochromocytoma and Paraganglioma | 0.436096365 |
| PRLHR | | |
| NOT-EPCAM AND NOT-ROR2 | Uveal Melanoma | 0.434834357 |
| AND TRPM1 | | |
| B4GALNT1 AND KREMEN2 | Testicular Germ Cell Tumors | 0.433962264 |
| NOT-ROR1 AND MC1R | Skin Cutaneous Melanoma | 0.43373494 |
| LYPD1 AND NOT-MET AND | Glioblastoma Multiforme | 0.433705841 |
| SLC1A3 | | |
| CD72 AND CD80 AND P2RX5 | Lymphoid Neoplasm Diffuse Large B-cell | 0.432432432 |
| | Lymphoma | |
| MUC16 AND L1CAM | Ovarian Serous Cystadenocarcinoma | 0.431654676 |
| KREMEN2 AND NOT-ERBB2 | Thymoma | 0.431372549 |
| NOT-KDR AND CHRNA5 | Lung Squamous Cell Carcinoma | 0.429780034 |
| GPR19 AND NOT-LIFR AND | Skin Cutaneous Melanoma | 0.429678968 |
| NOT-MUC1 | | |
| NOT-FAP AND NOT-L1CAM AND | Acute Myeloid Leukemia | 0.429175856 |
| RHAG | | |
| GPR19 AND GRM2 AND NOT- | Testicular Germ Cell Tumors | 0.429011866 |
| MUC1 | | |
| NOT-GPC3 AND GPR35 AND | Colon Adenocarcinoma | 0.424627359 |
| NOX1 | | |
| NOT-EGFR AND MSLN | Pancreatic Adenocarcinoma | 0.424437299 |
| SLCO5A1 AND NOT-ERBB2 | Thymoma | 0.423076923 |
| FAP AND B4GALNT1 | Pancreatic Adenocarcinoma | 0.421875 |
| NOT-PTH1R AND CD80 | Stomach Adenocarcinoma | 0.421416235 |
| CA9 AND PRR7 AND NOT-SELP | Ovarian Serous Cystadenocarcinoma | 0.421373361 |
| NOT-GPC3 AND P2RX5 AND | Lymphoid Neoplasm Diffuse Large B-cell | 0.421052632 |
| NOT-ROR1 | Lymphoma | |
| NOT-MET AND SLC4A8 | Pheochromocytoma and Paraganglioma | 0.420841683 |
| NOT-GPC3 AND GRIN2D AND | Colon Adenocarcinoma | 0.420245211 |
| NOX1 | | |
| ABCC4 AND FOLH1 AND | Prostate Adenocarcinoma | 0.419786137 |
| PMEPA1 | | |
| OR2B6 AND NOT-EGFR | Uterine Corpus Endometrial Carcinoma | 0.419753086 |
| NOT-PROM1 AND IL13RA2 | Pheochromocytoma and Paraganglioma | 0.419006479 |
| CLDN18 AND CEACAM5 | Stomach Adenocarcinoma | 0.417149479 |
| NOT-MUC1 AND MET | Uveal Melanoma | 0.413793103 |
| NOT-EPCAM AND MET | Uveal Melanoma | 0.412371134 |
| BSG AND NOT-GPC3 | Kidney Renal Papillary Cell Carcinoma | 0.412162162 |
| OR2B6 AND NOT-AXL | Uterine Corpus Endometrial Carcinoma | 0.409937888 |
| NOT-PROM1 AND GRIN2D | Bladder Urothelial Carcinoma | 0.40952381 |
| NOT-FOLH1 AND LTB4R | Thymoma | 0.407407407 |
| NOT-GHR AND BSG | Bladder Urothelial Carcinoma | 0.405594406 |
| GPR19 AND KREMEN2 AND | Testicular Germ Cell Tumors | 0.404040404 |
| NOT-MUC1 | | |
| NOT-ERBB2 AND L1CAM AND | Pheochromocytoma and Paraganglioma | 0.403499131 |
| NOT-MUC1 | | |
| FAP AND CA9 | Stomach Adenocarcinoma | 0.402898551 |
| CA9 AND KISS1R AND NOT- | Mesothelioma | 0.402488526 |
| VIPR1 | | |
| CSPG5 AND NOT-EPHA2 AND | Brain Lower Grade Glioma | 0.402428886 |
| NOT-MUC1 | | |
| CA9 AND KISS1R AND NOT- | Mesothelioma | 0.401455672 |
| TSPAN12 | | |
| TNFRSF9 AND NOT-TGFBR3 | Lung Adenocarcinoma | 0.400479616 |
| CD37 AND CD70 AND TLR9 | Lymphoid Neoplasm Diffuse Large B-cell | 0.4 |
| | Lymphoma | |
| NOT-EPCAM AND NOT-FOLH1 | Acute Myeloid Leukemia | 0.399685651 |
| AND GAPT | | |
| NOT-EPCAM AND BSG | Glioblastoma Multiforme | 0.398963731 |
| CD80 AND NOT-EGFR | Breast Invasive Carcinoma | 0.398284314 |
| CD8B AND NOT-L1CAM AND | Thymoma | 0.39816299 |
| SLCO5A1 | | |
| KREMEN2 AND NOT-PROM1 | Head and Neck Squamous Cell Carcinoma | 0.398010453 |
| AND ULBP2 | | |
| CHRNA3 AND BSG | Pheochromocytoma and Paraganglioma | 0.397435897 |
| CD8B AND NOT-L1CAM AND | Thymoma | 0.396256054 |
| NOT-SLC1A1 | | |
| NOT-KDR AND CNIH2 | Breast Invasive Carcinoma | 0.39255814 |
| NOT-EPHA2 AND OR51E2 AND | Prostate Adenocarcinoma | 0.392295597 |
| PODXL2 | | |
| ABCC4 AND NOT-EPHA2 AND | Prostate Adenocarcinoma | 0.392198392 |
| OR51E2 | | |
| GABRG2 AND BSG | Testicular Germ Cell Tumors | 0.392156863 |
| FAP AND PRR7 | Uterine Carcinosarcoma | 0.391304348 |
| NOT-EPCAM AND NOT-ROR2 | Uveal Melanoma | 0.39116533 |
| AND SLC6A17 | | |
| NOT-EPCAM AND NOT-LEPR | Skin Cutaneous Melanoma | 0.390834695 |
| AND SLC7A5 | | |
| KREMEN2 AND NOT-L1CAM | Thymoma | 0.390616681 |
| AND NOT-MUC1 | | |
| NOT-SEMA4A AND B4GALNT1 | Adrenocortical Carcinoma | 0.390243902 |
| MET AND NOT-GPC3 | Kidney Renal Papillary Cell Carcinoma | 0.390243902 |
| ATP8B3 AND NOT-MUC1 AND | Adrenocortical Carcinoma | 0.390243902 |
| NOT-VIPR1 | | |
| NOT-LEPR AND FAP | Breast Invasive Carcinoma | 0.389363723 |
| NOT-ROR2 AND ERBB2 | Kidney Renal Papillary Cell Carcinoma | 0.38790932 |
| NOT-ADCY5 AND GABRD AND | Thyroid Carcinoma | 0.387527446 |
| NOT-GPC3 | | |
| BSG AND CACNG2 | Pheochromocytoma and Paraganglioma | 0.387096774 |
| BSG AND L1CAM | Pheochromocytoma and Paraganglioma | 0.387096774 |
| NOT-EGFR AND L1CAM | Uveal Melanoma | 0.386363636 |
| SLC2A1 AND B4GALNT1 | Lung Squamous Cell Carcinoma | 0.386052304 |
| NOT-MUC1 AND RHAG AND | Acute Myeloid Leukemia | 0.385500731 |
| NOT-ROR1 | | |
| NOT-MUC1 AND PAQR9 AND | Liver Hepatocellular Carcinoma | 0.38543364 |
| NOT-SLC15A2 | | |
| NOT-EPCAM AND NOT-MET | Glioblastoma Multiforme | 0.384917599 |
| AND NTSR2 | | |
| NOT-GPC3 AND KREMEN2 AND | Thymoma | 0.384174324 |
| NOT-MUC1 | | |
| NOT-EPHA2 AND MC1R | Adrenocortical Carcinoma | 0.382978723 |
| NOT-MUC1 AND PAQR9 AND | Liver Hepatocellular Carcinoma | 0.382449851 |
| NOT-TSPAN2 | | |
| NOT-KDR AND PRR7 | Uterine Corpus Endometrial Carcinoma | 0.382022472 |
| L1CAM AND NKAIN1 AND NOT- | Pheochromocytoma and Paraganglioma | 0.381634087 |
| PROM1 | | |
| TNFRSF9 AND NOT-ABCB1 | Lung Adenocarcinoma | 0.381368268 |
| NOT-MUC1 AND ERBB2 | Prostate Adenocarcinoma | 0.381246144 |
| NKAIN1 AND NOT-CD274 | Uterine Carcinosarcoma | 0.380952381 |
| BSG AND GRIN2D | Stomach Adenocarcinoma | 0.380645161 |
| L1CAM AND PRLHR AND NOT- | Pheochromocytoma and Paraganglioma | 0.380315609 |
| PROM1 | | |
| ST3GAL5 AND NOT-GPC3 | Thyroid Carcinoma | 0.378986867 |
| NOT-MET AND B4GALNT1 | Breast Invasive Carcinoma | 0.378809869 |
| KREMEN2 AND LY6K AND NOT- | Head and Neck Squamous Cell Carcinoma | 0.378229871 |
| PROM1 | | |
| ABCC4 AND NOT-EPHA2 AND | Prostate Adenocarcinoma | 0.375928439 |
| FOLH1 | | |
| MUC1 AND NOT-KDR | Uterine Corpus Endometrial Carcinoma | 0.373056995 |
| NOT-GPC3 AND SLC7A5 AND | Skin Cutaneous Melanoma | 0.373027921 |
| TRPM1 | | |
| NOT-EPHA2 AND FOLH1 AND | Prostate Adenocarcinoma | 0.369918699 |
| KCNN2 | | |
| B4GALNT1 AND NOT-EPCAM | Brain Lower Grade Glioma | 0.369617881 |
| AND NOT-MET | | |
| KISS1R AND NOT-AXL | Uterine Corpus Endometrial Carcinoma | 0.367816092 |
| FAP AND NOT-EGFR | Uterine Carcinosarcoma | 0.367346939 |
| NOT-EGFR AND MC1R AND | Skin Cutaneous Melanoma | 0.367317058 |
| NOT-MUC1 | | |
| CA9 AND GRIN2D AND PRR7 | Esophageal Carcinoma | 0.366171368 |
| BSG AND GRIN2D | Bladder Urothelial Carcinoma | 0.366071429 |
| NOT-GPC3 AND KCNQ2 AND | Glioblastoma Multiforme | 0.365840351 |
| RGR | | |
| NOT-LYVE1 AND NOT-PROM1 | Prostate Adenocarcinoma | 0.365422833 |
| AND TRPM8 | | |
| CNIH2 AND NOT-EPHA2 AND | Brain Lower Grade Glioma | 0.364710529 |
| NOT-MUC1 | | |
| CA9 AND NOT-GPC3 AND NOX1 | Colon Adenocarcinoma | 0.363336945 |
| CA9 AND NOT-GPC3 AND | Rectum Adenocarcinoma | 0.362034966 |
| GPR35 | | |
| B4GALNT1 AND GRIN2D | Pancreatic Adenocarcinoma | 0.36123348 |
| NOT-VIPR1 AND ERBB2 | Kidney Renal Papillary Cell Carcinoma | 0.36101083 |
| NOT-EGFR AND NOT-EPCAM | Skin Cutaneous Melanoma | 0.360842613 |
| AND MC1R | | |
| NOT-ABCA8 AND NOT-KDR AND | Cervical Squamous Cell Carcinoma and | 0.360757313 |
| KREMEN2 | Endocervical Adenocarcinoma | |
| NOT-EPCAM AND BSG | Skin Cutaneous Melanoma | 0.360655738 |
| B4GALNT1 AND BSG | Testicular Germ Cell Tumors | 0.360655738 |
| FOLH1 AND KCNN2 AND | Prostate Adenocarcinoma | 0.3599182 |
| PMEPA1 | | |
| GRIN1 AND NOT-PROM1 AND | Prostate Adenocarcinoma | 0.358954211 |
| TRPM8 | | |
| CA9 AND LY6K AND ULBP2 | Head and Neck Squamous Cell Carcinoma | 0.35619976 |
| CA9 AND KREMEN2 AND NOT- | Uterine Carcinosarcoma | 0.356078124 |
| SELP | | |
| CA9 AND NOT-GPC3 AND | Colon Adenocarcinoma | 0.355551579 |
| GPR35 | | |
| TNFRSF9 AND NOT-FOLR1 | Head and Neck Squamous Cell Carcinoma | 0.354806739 |
| CA9 AND FAP AND KISS1R | Mesothelioma | 0.354534954 |
| CA9 AND FZD2 AND KREMEN2 | Uterine Carcinosarcoma | 0.353927948 |
| KREMEN2 AND LYPD1 AND | Ovarian Serous Cystadenocarcinoma | 0.353693924 |
| MSLN | | |
| CA9 AND GRIN2D AND PRR7 | Stomach Adenocarcinoma | 0.353349213 |
| NOT-ABCA8 AND EPCAM AND | Colon Adenocarcinoma | 0.353230905 |
| EPHB2 | | |
| CD72 AND NOT-EPCAM AND | Lymphoid Neoplasm Diffuse Large B-cell | 0.352941176 |
| NOT-KDR | Lymphoma | |
| CELSR3 AND EPCAM AND | Rectum Adenocarcinoma | 0.352576855 |
| LY6G6D | | |
| NOT-ABCG2 AND CA9 AND FAP | Mesothelioma | 0.350490726 |
| CA9 AND NOT-L1CAM | Lung Adenocarcinoma | 0.350282486 |
| B4GALNT1 AND NOT-EPCAM | Brain Lower Grade Glioma | 0.350069765 |
| AND NOT-EPHA2 | | |
| NOT-FOLR1 AND B4GALNT1 | Prostate Adenocarcinoma | 0.350030175 |
| MC1R AND NOT-AXL | Adrenocortical Carcinoma | 0.35 |
| CA9 AND PRR7 AND NOT-PTH1R | Esophageal Carcinoma | 0.34989566 |
| NOT-ABCA8 AND EPCAM AND | Colon Adenocarcinoma | 0.349335242 |
| GPA33 | | |
| ADAM12 AND NOT-EPCAM AND | Sarcoma | 0.34791126 |
| NOT-F11R | | |
| CA9 AND CELSR3 AND NOT- | Cervical Squamous Cell Carcinoma and | 0.347704279 |
| LYVE1 | Endocervical Adenocarcinoma | |
| FAP AND SLC7A5 | Head and Neck Squamous Cell Carcinoma | 0.345924453 |
| NOT-FOLR1 AND MLANA AND | Skin Cutaneous Melanoma | 0.345168462 |
| NOT-SLC40A1 | | |
| CD70 AND NOT-GPC3 | Head and Neck Squamous Cell Carcinoma | 0.344433873 |
| CELSR3 AND EPCAM AND | Rectum Adenocarcinoma | 0.34427892 |
| GPA33 | | |
| EPCAM AND NOT-L1CAM | Thyroid Carcinoma | 0.344116268 |
| NOT-EPCAM AND NOT-KDR | Lymphoid Neoplasm Diffuse Large B-cell | 0.342857143 |
| AND LRMP | Lymphoma | |
| NOT-EPCAM AND MMP14 AND | Sarcoma | 0.342766365 |
| NOT-VIPR1 | | |
| NOT-EPHA2 AND B4GALNT1 | Breast Invasive Carcinoma | 0.34267101 |
| MLANA AND NOT-PROM1 AND | Skin Cutaneous Melanoma | 0.341019562 |
| NOT-SLC40A1 | | |
| NOT-ABCA8 AND NOT-GPC3 | Thymoma | 0.3407568 |
| AND LAT | | |
| NOT-VIPR1 AND B4GALNT1 | Adrenocortical Carcinoma | 0.340425532 |
| CNIH2 AND NOT-GPC3 AND | Brain Lower Grade Glioma | 0.339918723 |
| NOT-ROR2 | | |
| MUC1 AND NOT-L1CAM | Lung Adenocarcinoma | 0.339440694 |
| NOT-FOLH1 AND CEACAM5 | Cervical Squamous Cell Carcinoma and | 0.338931298 |
| | Endocervical Adenocarcinoma | |
| NOT-FOLR1 AND NOT-MET AND | Glioblastoma Multiforme | 0.338260981 |
| NTSR2 | | |
| CA9 AND CELSR3 AND NOT- | Cervical Squamous Cell Carcinoma and | 0.338016035 |
| EDNRB | Endocervical Adenocarcinoma | |
| KDR AND NOT-ERBB2 | Kidney Renal Clear Cell Carcinoma | 0.336065574 |
| CA9 AND PRR7 AND NOT-PTH1R | Stomach Adenocarcinoma | 0.334803442 |
| CA9 AND NOT-ERBB2 | Glioblastoma Multiforme | 0.334128878 |
| NOT-ROR2 AND KREMEN2 | Skin Cutaneous Melanoma | 0.333333333 |
| NOT-EGFR AND CA9 | Uveal Melanoma | 0.333333333 |
| CD80 AND NOT-ERBB2 | Acute Myeloid Leukemia | 0.333333333 |
| NOT-GPC3 AND RGR AND NOT- | Brain Lower Grade Glioma | 0.33257969 |
| ROR2 | | |
| FAP AND CD80 | Lung Adenocarcinoma | 0.33046202 |
| NOT-F11R AND B4GALNT1 | Sarcoma | 0.328638498 |
| MET AND NOT-L1CAM | Thyroid Carcinoma | 0.328086164 |
| GABRD AND NOT-GPC3 AND | Thyroid Carcinoma | 0.32792997 |
| NOT-PROM1 | | |
| MUC16 AND NOT-GPC3 | Cervical Squamous Cell Carcinoma and | 0.327868852 |
| | Endocervical Adenocarcinoma | |
| NOT-CD274 AND KREMEN2 | Ovarian Serous Cystadenocarcinoma | 0.327272727 |
| NOT-FOLR1 AND NOT-PROM1 | Uveal Melanoma | 0.327221661 |
| AND TRPM1 | | |
| LYPD1 AND MSLN AND NOT- | Ovarian Serous Cystadenocarcinoma | 0.324317755 |
| RAMP3 | | |
| CA9 AND SLC7A5 AND NOT- | Head and Neck Squamous Cell Carcinoma | 0.322954859 |
| TSPAN12 | | |
| NOT-FOLR1 AND IL13RA2 AND | Pheochromocytoma and Paraganglioma | 0.322743638 |
| PTPRN | | |
| NOT-EPCAM AND GPR19 AND | Skin Cutaneous Melanoma | 0.320934426 |
| NOT-MUC1 | | |
| NOT-EPHA2 AND BSG | Prostate Adenocarcinoma | 0.320836966 |
| CELSR3 AND NOT-KDR | Cervical Squamous Cell Carcinoma and | 0.320126783 |
| | Endocervical Adenocarcinoma | |
| NOT-MET AND SCARB1 | Adrenocortical Carcinoma | 0.318181818 |
| NOT-MUC1 AND BSG | Brain Lower Grade Glioma | 0.318107667 |
| NOT-F11R AND FAP AND NOT- | Sarcoma | 0.316771089 |
| SCNN1A | | |
| NOT-L1CAM AND SLC22A9 AND | Liver Hepatocellular Carcinoma | 0.315068645 |
| SLC30A10 | | |
| HCN2 AND NOT-MET AND NOT- | Adrenocortical Carcinoma | 0.314518533 |
| PROM1 | | |
| NOT-ABCA8 AND FAP AND NOT- | Breast Invasive Carcinoma | 0.313774884 |
| LYVE1 | | |
| FAP AND HAS1 AND NOT- | Mesothelioma | 0.31329683 |
| TMEM30B | | |
| NOT-KDR AND MMP24 | Kidney Renal Papillary Cell Carcinoma | 0.313253012 |
| NOT-PROM1 AND MC1R | Mesothelioma | 0.313253012 |
| GRM2 AND NOT-EGFR | Testicular Germ Cell Tumors | 0.312328767 |
| SLC2A1 AND NOT-PROM1 | Bladder Urothelial Carcinoma | 0.312258065 |
| FOLH1 AND NOT-FOLR1 | Liver Hepatocellular Carcinoma | 0.311881188 |
| NOT-ABCB1 AND CA9 AND | Lung Squamous Cell Carcinoma | 0.311550397 |
| SLC2A1 | | |
| FAP AND CELSR3 | Esophageal Carcinoma | 0.310810811 |
| CA9 AND NOT-GPC3 | Glioblastoma Multiforme | 0.310526316 |
| GABRD AND NOT-L1CAM AND | Liver Hepatocellular Carcinoma | 0.310042161 |
| SLC30A10 | | |
| EPCAM AND NOT-MET | Prostate Adenocarcinoma | 0.310036784 |
| NOT-FOLR1 AND GRIN2C AND | Adrenocortical Carcinoma | 0.309266966 |
| NOT-SEMA4A | | |
| NOT-PROM1 AND ERBB2 | Thyroid Carcinoma | 0.308392315 |
| NOT-ITGA9 AND ERBB2 | Kidney Renal Papillary Cell Carcinoma | 0.307984791 |
| NOT-ROR1 AND LTB4R | Thymoma | 0.307692308 |
| NOT-TNFRSF9 AND HCN2 | Kidney Chromophobe | 0.307692308 |
| FAP AND LY6K | Head and Neck Squamous Cell Carcinoma | 0.307692308 |
| NOT-GPC3 AND KCNQ2 AND | Glioblastoma Multiforme | 0.307692308 |
| NOT-LSR | | |
| HCN2 AND NOT-MET AND NOT- | Adrenocortical Carcinoma | 0.307692308 |
| SEMA4A | | |
| NOT-L1CAM AND NOT-MUC1 | Liver Hepatocellular Carcinoma | 0.307538741 |
| AND PAQR9 | | |
| NOT-PROM1 AND FOLH1 | Liver Hepatocellular Carcinoma | 0.307304786 |
| NOT-FOLR1 AND IL13RA2 AND | Pheochromocytoma and Paraganglioma | 0.307258881 |
| KCNQ2 | | |
| NOT-FOLR1 AND NOT-PROM1 | Uveal Melanoma | 0.305652834 |
| AND SLC6A17 | | |
| NOT-MET AND ROR2 | Pheochromocytoma and Paraganglioma | 0.305185185 |
| GABRD AND NOT-ROR1 | Thyroid Carcinoma | 0.303763441 |
| NOT-EGFR AND L1CAM AND | Pheochromocytoma and Paraganglioma | 0.302824126 |
| NOT-MET | | |
| NOT-ABCA8 AND NOT-ABCB1 | Lung Squamous Cell Carcinoma | 0.302412032 |
| AND CA9 | | |
| B4GALNT1 AND NOT-SCNN1A | Sarcoma | 0.301886792 |
| AND NOT-VIPR1 | | |
| B4GALNT1 AND NOT-CLDN7 | Sarcoma | 0.301808254 |
| AND NOT-SORL1 | | |
| NOT-MET AND IL13RA2 | Pheochromocytoma and Paraganglioma | 0.300484653 |
| CEACAM5 AND NOT-GPC3 | Stomach Adenocarcinoma | 0.300455235 |
| LRP8 AND NOT-KDR | Lung Squamous Cell Carcinoma | 0.300283286 |
| NOT-ADCY5 AND GABBR2 AND | Thyroid Carcinoma | 0.300147735 |
| NOT-PROM1 | | |
| CA9 AND NOT-FOLH1 | Kidney Renal Papillary Cell Carcinoma | 0.29972752 |
| NOT-ABCA8 AND CEACAM5 | Rectum Adenocarcinoma | 0.299047853 |
| AND EPHB2 | | |
| NOT-MUC1 AND BSG | Pheochromocytoma and Paraganglioma | 0.297297297 |
| B4GALNT1 AND NOT-EPCAM | Sarcoma | 0.297085861 |
| AND NOT-VIPR1 | | |
| CA9 AND MSLN AND PRR7 | Ovarian Serous Cystadenocarcinoma | 0.296737818 |
| CA9 AND CELSR3 AND NOT-KDR | Cervical Squamous Cell Carcinoma and | 0.295846342 |
| | Endocervical Adenocarcinoma | |
| NOT-ABCA8 AND NOT-ADRB2 | Rectum Adenocarcinoma | 0.295542679 |
| AND CEACAM5 | | |
| EPHA2 AND NOT-ROR1 | Bladder Urothelial Carcinoma | 0.295454545 |
| NOT-MUC1 AND AXL | Brain Lower Grade Glioma | 0.294642857 |
| B4GALNT1 AND NOT-EPCAM | Sarcoma | 0.294228567 |
| AND MMP14 | | |
| NOT-ABCA8 AND FAP AND NOT- | Breast Invasive Carcinoma | 0.294142737 |
| SLC4A4 | | |
| NOT-ROR1 AND PRR7 | Uterine Corpus Endometrial Carcinoma | 0.294117647 |
| CEACAM5 AND NOT-GPC3 | Lung Adenocarcinoma | 0.293885602 |
| NOT-ABCB1 AND BSG | Lung Adenocarcinoma | 0.293814433 |
| CA9 AND KREMEN2 AND MSLN | Ovarian Serous Cystadenocarcinoma | 0.293761868 |
| EPCAM AND NOT-GPC3 | Prostate Adenocarcinoma | 0.293103448 |
| CA9 AND FAP AND NOT-PROM1 | Mesothelioma | 0.292284308 |
| CD8B AND NOT-L1CAM AND | Thymoma | 0.29222464 |
| NOT-PROM1 | | |
| NOT-ERBB2 AND AXL | Sarcoma | 0.291139241 |
| NOT-MET AND CA9 | Ovarian Serous Cystadenocarcinoma | 0.289890378 |
| GABBR2 AND NOT-GPC3 AND | Thyroid Carcinoma | 0.289323254 |
| NOT-PROM1 | | |
| CD70 AND NOT-IL13RA2 | Kidney Renal Papillary Cell Carcinoma | 0.287958115 |
| CA9 AND NOT-SLC2A4 | Pancreatic Adenocarcinoma | 0.286919831 |
| CRB2 AND NOT-PROM1 AND | Mesothelioma | 0.286690867 |
| NOT-VIPR1 | | |
| CD8B AND NOT-GPC3 AND | Thymoma | 0.285842111 |
| NOT-PROM1 | | |
| SLCO5A1 AND NOT-B4GALNT1 | Thymoma | 0.285714286 |
| GPR19 AND BSG | Testicular Germ Cell Tumors | 0.285714286 |
| NOT-GPC3 AND KREMEN2 AND | Skin Cutaneous Melanoma | 0.285714286 |
| TRPM1 | | |
| KREMEN2 AND L1CAM AND | Skin Cutaneous Melanoma | 0.285714286 |
| NOT-PARM1 | | |
| NOT-MET AND BSG | Prostate Adenocarcinoma | 0.284741917 |
| LRP8 AND NOT-ROR1 | Lung Squamous Cell Carcinoma | 0.283030683 |
| NOT-FOLR1 AND NOT-GPC3 | Skin Cutaneous Melanoma | 0.282301832 |
| AND GPR19 | | |
| B4GALNT1 AND NOT-EPCAM | Glioblastoma Multiforme | 0.281206556 |
| AND NOT-MET | | |
| NOT-CD274 AND ERBB2 | Prostate Adenocarcinoma | 0.280178838 |
| NOT-FOLR1 AND NOT-GPC3 | Skin Cutaneous Melanoma | 0.28003032 |
| AND MLANA | | |
| ATP8B3 AND FAP AND UPK3B | Mesothelioma | 0.278468406 |
| IL13RA2 AND NOT-GPC3 | Glioblastoma Multiforme | 0.278409091 |
| NOT-EGFR AND KREMEN2 | Skin Cutaneous Melanoma | 0.275862069 |
| B4GALNT1 AND NOT-ERBB2 | Adrenocortical Carcinoma | 0.275862069 |
| CA9 AND CD70 AND FAP | Mesothelioma | 0.275862069 |
| NOT-MUC1 AND BSG | Testicular Germ Cell Tumors | 0.274509804 |
| NOT-GGT1 AND NOT-GPC3 AND | Adrenocortical Carcinoma | 0.274005551 |
| GRIN2C | | |
| NOT-ROR1 AND CELSR3 | Cervical Squamous Cell Carcinoma and | 0.271954674 |
| | Endocervical Adenocarcinoma | |
| NOT-MUC1 AND B4GALNT1 | Liver Hepatocellular Carcinoma | 0.271903323 |
| NOT-LGR6 AND MET | Kidney Renal Clear Cell Carcinoma | 0.269966254 |
| CD70 AND GABRD AND NOT- | Kidney Renal Clear Cell Carcinoma | 0.269882188 |
| VIPR1 | | |
| ATP8B3 AND CD70 AND UPK3B | Mesothelioma | 0.269591014 |
| CD70 AND MUC16 | Cervical Squamous Cell Carcinoma and | 0.268041237 |
| | Endocervical Adenocarcinoma | |
| NOT-L1CAM AND NOT-MUC1 | Liver Hepatocellular Carcinoma | 0.267094828 |
| AND SLC30A10 | | |
| B4GALNT1 AND NOT-EPCAM | Glioblastoma Multiforme | 0.265770851 |
| AND NOT-FOLR1 | | |
| NOT-VIPR1 AND MET | Kidney Renal Clear Cell Carcinoma | 0.265486726 |
| CA9 AND KREMEN2 AND NOT- | Head and Neck Squamous Cell Carcinoma | 0.2637398 |
| PROM1 | | |
| MET AND NOT-L1CAM | Liver Hepatocellular Carcinoma | 0.263548203 |
| NOT-PROM1 AND CEACAM5 | Lung Squamous Cell Carcinoma | 0.263240692 |
| CA9 AND NOT-PROM1 AND | Head and Neck Squamous Cell Carcinoma | 0.262945646 |
| ULBP2 | | |
| CA9 AND CELSR3 AND NOT- | Pancreatic Adenocarcinoma | 0.262601361 |
| SLC2A4 | | |
| BSG AND FFAR1 | Pancreatic Adenocarcinoma | 0.261437908 |
| CA9 AND CELSR3 AND GRIN2D | Pancreatic Adenocarcinoma | 0.260742758 |
| TNFRSF9 AND FAP | Lung Adenocarcinoma | 0.259757739 |
| BSG AND NOT-GPC3 | Colon Adenocarcinoma | 0.259520451 |
| NOT-LYVE1 AND BSG | Uterine Corpus Endometrial Carcinoma | 0.258992806 |
| FAP AND PTPRH AND PTPRN | Pancreatic Adenocarcinoma | 0.258834432 |
| NOT-ABCA8 AND BSG | Cervical Squamous Cell Carcinoma and | 0.258741259 |
| | Endocervical Adenocarcinoma | |
| CEACAM5 AND NOT-GPC3 | Rectum Adenocarcinoma | 0.258278146 |
| CALHM3 AND FAP AND PTPRN | Pancreatic Adenocarcinoma | 0.257506376 |
| NOT-TNFRSF9 AND KREMEN2 | Cervical Squamous Cell Carcinoma and | 0.257206208 |
| | Endocervical Adenocarcinoma | |
| NOT-ABCG2 AND NOT-KIT AND | Mesothelioma | 0.256163202 |
| MSLN | | |
| NOT-GPC3 AND NOT-KDR AND | Kidney Renal Papillary Cell Carcinoma | 0.255327006 |
| SLC3A1 | | |
| NOT-AOC3 AND BSG | Uterine Corpus Endometrial Carcinoma | 0.255319149 |
| CD70 AND KISS1R AND NOT- | Kidney Renal Clear Cell Carcinoma | 0.255205339 |
| VIPR1 | | |
| GRM2 AND NOT-MET | Testicular Germ Cell Tumors | 0.253638254 |
| NOT-EGFR AND GRIN2D | Testicular Germ Cell Tumors | 0.253333333 |
| NOT-CEACAM1 AND GABRD | Thyroid Carcinoma | 0.253308129 |
| AND NOT-GPC3 | | |
| NOT-EPHA2 AND NOT-FOLR1 | Adrenocortical Carcinoma | 0.253172306 |
| AND HCN2 | | |
| CD70 AND HAS1 AND NOT- | Mesothelioma | 0.25198832 |
| PROM1 | | |
| MUC1 AND B4GALNT1 | Lung Adenocarcinoma | 0.251068376 |
| NOT-MUC1 AND B4GALNT1 | Adrenocortical Carcinoma | 0.25 |
| L1CAM AND MLANA AND NOT- | Skin Cutaneous Melanoma | 0.249724513 |
| PROM1 | | |
| NOT-PTH1R AND BSG | Stomach Adenocarcinoma | 0.248979592 |
| CLDN15 AND NOT-EPCAM AND | Mesothelioma | 0.248555014 |
| NOT-TSPAN12 | | |
| FAP AND NOT-PROM1 | Head and Neck Squamous Cell Carcinoma | 0.248210024 |
| CA9 AND NOT-GPC3 | Stomach Adenocarcinoma | 0.247191011 |
| NOT-KDR AND B4GALNT1 | Head and Neck Squamous Cell Carcinoma | 0.246656761 |
| CEACAM5 AND GPA33 AND | Colon Adenocarcinoma | 0.24540388 |
| NOT-SFRP1 | | |
| ROR2 AND CD70 | Mesothelioma | 0.242424242 |
| NOT-ABCA8 AND NOT-PROM1 | Thymoma | 0.241610738 |
| AND SLCO5A1 | | |
| B4GALNT1 AND NOT-EPCAM | Sarcoma | 0.241304587 |
| AND FAP | | |
| GPR143 AND L1CAM AND NOT- | Skin Cutaneous Melanoma | 0.24065118 |
| PROM1 | | |
| CEACAM5 AND EPCAM AND | Rectum Adenocarcinoma | 0.240413964 |
| NOT-LIFR | | |
| NOT-ABCA8 AND NOT-ADRB2 | Cholangiocarcinoma | 0.239026022 |
| AND CA9 | | |
| NOT-KDR AND GRIN2D | Rectum Adenocarcinoma | 0.23880597 |
| NOT-TMEM30B AND BSG | Skin Cutaneous Melanoma | 0.238095238 |
| NOT-ABCA8 AND CA9 AND | Cholangiocarcinoma | 0.237925219 |
| NOT-SCN4B | | |
| NOT-ROR1 AND B4GALNT1 | Head and Neck Squamous Cell Carcinoma | 0.236263736 |
| GPR35 AND NOT-KDR | Rectum Adenocarcinoma | 0.235294118 |
| NOT-EGFR AND CA9 | Testicular Germ Cell Tumors | 0.233333333 |
| NOT-FOLR1 AND CEACAM5 | Esophageal Carcinoma | 0.231660232 |
| CEACAM5 AND NOT-PTH1R | Colon Adenocarcinoma | 0.231012815 |
| AND NOT-SFRP1 | | |
| CEACAM5 AND EPCAM AND | Rectum Adenocarcinoma | 0.228256789 |
| GPR35 | | |
| CA9 AND EPCAM AND NOT- | Colon Adenocarcinoma | 0.226291468 |
| GPC3 | | |
| NOT-KDR AND BSG | Uterine Corpus Endometrial Carcinoma | 0.225641026 |
| IL13RA2 AND NOT-MET AND | Pheochromocytoma and Paraganglioma | 0.225465906 |
| NOT-PROM1 | | |
| CA9 AND EPCAM AND NOT- | Rectum Adenocarcinoma | 0.225265425 |
| GPC3 | | |
| NOT-ERBB2 AND EMP3 | Sarcoma | 0.224719101 |
| NOT-ROR1 AND CELSR3 | Lung Adenocarcinoma | 0.223953262 |
| CA9 AND CEACAM5 AND NOT- | Rectum Adenocarcinoma | 0.221642363 |
| GPC3 | | |
| FAP AND NOT-FOLR1 AND NOT- | Sarcoma | 0.220530819 |
| SCNN1A | | |
| NOT-CLDN7 AND FAP AND NOT- | Sarcoma | 0.219908352 |
| FOLR1 | | |
| NOT-KDR AND PRR7 | Esophageal Carcinoma | 0.219178082 |
| FAP AND NOT-KDR | Head and Neck Squamous Cell Carcinoma | 0.218023256 |
| BSG AND NOT-GPC3 | Head and Neck Squamous Cell Carcinoma | 0.216666667 |
| NOT-ROR2 AND CD70 | Kidney Renal Papillary Cell Carcinoma | 0.214814815 |
| NOT-KIT AND MSLN AND NOT- | Mesothelioma | 0.214285714 |
| XK | | |
| MSLN AND NOT-PROM1 AND | Mesothelioma | 0.214063765 |
| NOT-TMEM30B | | |
| BSG AND CD70 | Head and Neck Squamous Cell Carcinoma | 0.213031161 |
| CA9 AND KISS1R AND NOT- | Kidney Renal Clear Cell Carcinoma | 0.212072373 |
| SCARA5 | | |
| CA9 AND GABRD AND NOT- | Kidney Renal Clear Cell Carcinoma | 0.21165805 |
| SCARA5 | | |
| NOT-ROR2 AND MMP24 | Kidney Renal Papillary Cell Carcinoma | 0.211072664 |
| NOT-PROM1 AND BSG | Mesothelioma | 0.210526316 |
| NOT-MSLN AND GPC3 | Liver Hepatocellular Carcinoma | 0.210526316 |
| NOT-ERBB2 AND SCARB1 AND | Adrenocortical Carcinoma | 0.210526316 |
| NOT-ST14 | | |
| NOT-KDR AND BSG | Colon Adenocarcinoma | 0.20977354 |
| MSLN AND NOT-IL13RA2 | Cervical Squamous Cell Carcinoma and | 0.209424084 |
| | Endocervical Adenocarcinoma | |
| NOT-EGFR AND CD70 | Lymphoid Neoplasm Diffuse Large B-cell | 0.209150327 |
| | Lymphoma | |
| CA9 AND NOT-GPC3 | Uterine Corpus Endometrial Carcinoma | 0.207865169 |
| TNFRSF9 AND B4GALNT1 | Lung Adenocarcinoma | 0.207455429 |
| CD70 AND NOT-CEACAM1 AND | Mesothelioma | 0.206896552 |
| HAS1 | | |
| CA9 AND CELSR3 AND FAP | Pancreatic Adenocarcinoma | 0.206713191 |
| NOT-FOLH1 AND AXL | Kidney Renal Papillary Cell Carcinoma | 0.20668693 |
| EPCAM AND NOT-GPC3 | Lung Adenocarcinoma | 0.20516129 |
| CEACAM5 AND NOT-IL13RA2 | Cervical Squamous Cell Carcinoma and | 0.204771372 |
| | Endocervical Adenocarcinoma | |
| CELSR3 AND NOT-KDR | Bladder Urothelial Carcinoma | 0.204220558 |
| B4GALNT1 AND BRCA1 | Esophageal Carcinoma | 0.204081633 |
| B4GALNT1 AND IL13RA2 AND | Pheochromocytoma and Paraganglioma | 0.203052132 |
| NOT-PROM1 | | |
| NOT-ROR1 AND CELSR3 | Bladder Urothelial Carcinoma | 0.202806122 |
| CA9 AND FAP AND PTPRH | Pancreatic Adenocarcinoma | 0.20228396 |
| FAP AND NOT-FOLH1 | Colon Adenocarcinoma | 0.201995012 |
| B4GALNT1 AND NOT-EPCAM | Sarcoma | 0.197710051 |
| AND NOT-FOLR1 | | |
| GPC3 AND NOT-L1CAM | Liver Hepatocellular Carcinoma | 0.195845697 |
| CEACAM5 AND NOT-IL13RA2 | Lung Squamous Cell Carcinoma | 0.194053208 |
| FAP AND NOT-ADRB2 | Cholangiocarcinoma | 0.193548387 |
| SLC7A11 AND BSG | Esophageal Carcinoma | 0.192893401 |
| NOT-KDR AND CEACAM5 | Rectum Adenocarcinoma | 0.189473684 |
| NOT-MUC1 AND B4GALNT1 | Prostate Adenocarcinoma | 0.18875502 |
| NOT-MET AND L1CAM | Brain Lower Grade Glioma | 0.186895811 |
| CEACAM5 AND EPCAM AND | Colon Adenocarcinoma | 0.186666495 |
| NOT-LIFR | | |
| NOT-GPC3 AND L1CAM | Brain Lower Grade Glioma | 0.18639329 |
| ADAM12 AND NOT-CEACAM1 | Sarcoma | 0.186186186 |
| AND FAP | | |
| EPCAM AND MSLN | Pancreatic Adenocarcinoma | 0.183745583 |
| NOT-FOLR1 AND CD70 | Sarcoma | 0.183098592 |
| TNFRSF9 AND NOT-FOLH1 | Stomach Adenocarcinoma | 0.182336182 |
| CELSR3 AND NOT-KDR | Stomach Adenocarcinoma | 0.182260024 |
| CA9 AND BSG | Mesothelioma | 0.181818182 |
| ROR2 AND NOT-ERBB2 | Thymoma | 0.181818182 |
| NOT-KDR AND GRIN2D | Pancreatic Adenocarcinoma | 0.181229773 |
| CA9 AND CD70 AND NOT-VIPR1 | Kidney Renal Clear Cell Carcinoma | 0.178072894 |
| BSG AND NOX1 | Rectum Adenocarcinoma | 0.177777778 |
| NOT-ABCA8 AND CEACAM5 | Colon Adenocarcinoma | 0.175699675 |
| AND EPCAM | | |
| KREMEN2 AND BSG | Esophageal Carcinoma | 0.175182482 |
| NOT-KDR AND BSG | Bladder Urothelial Carcinoma | 0.174365647 |
| BSG AND NOT-GPC3 | Bladder Urothelial Carcinoma | 0.1738437 |
| NOT-KDR AND FZD2 | Uterine Corpus Endometrial Carcinoma | 0.173611111 |
| NOT-MET AND ATP8B3 | Adrenocortical Carcinoma | 0.173333333 |
| CD80 AND NOT-MET | Acute Myeloid Leukemia | 0.173285199 |
| BSG AND NOT-AXL | Uterine Corpus Endometrial Carcinoma | 0.172248804 |
| NOT-EGFR AND ERBB2 | Breast Invasive Carcinoma | 0.172228202 |
| MUC1 AND NOT-AXL | Uterine Corpus Endometrial Carcinoma | 0.171990172 |
| CA9 AND NOT-GPC3 | Rectum Adenocarcinoma | 0.171990172 |
| NOT-EPHA2 AND CD80 | Testicular Germ Cell Tumors | 0.171929825 |
| NOT-MET AND CA9 | Testicular Germ Cell Tumors | 0.16988417 |
| NOT-PTGER3 AND BSG | Lung Adenocarcinoma | 0.168606301 |
| NOT-LYVE1 AND BSG | Cervical Squamous Cell Carcinoma and | 0.16856492 |
| | Endocervical Adenocarcinoma | |
| NOT-PROM1 AND ROR2 | Pheochromocytoma and Paraganglioma | 0.168148747 |
| ADAM12 AND BSG | Sarcoma | 0.166666667 |
| ATP8B3 AND NOT-MUC1 AND | Adrenocortical Carcinoma | 0.166666667 |
| SCARB1 | | |
| NOT-CD274 AND PTPRH | Pancreatic Adenocarcinoma | 0.164158687 |
| NOT-KDR AND BSG | Ovarian Serous Cystadenocarcinoma | 0.162393162 |
| NOT-ADRB2 AND B4GALNT1 | Cholangiocarcinoma | 0.162162162 |
| CA9 AND CEACAM5 AND NOT- | Colon Adenocarcinoma | 0.160951808 |
| GPC3 | | |
| NOT-F11R AND FAP | Sarcoma | 0.157549234 |
| NOT-CD274 AND P2RY11 | Uterine Corpus Endometrial Carcinoma | 0.156626506 |
| NOT-KDR AND BSG | Cervical Squamous Cell Carcinoma and | 0.155172414 |
| | Endocervical Adenocarcinoma | |
| CD70 AND MSLN AND NOT- | Mesothelioma | 0.155049753 |
| PROM1 | | |
| CD70 AND CD80 AND NOT- | Lymphoid Neoplasm Diffuse Large B-cell | 0.154929577 |
| EGFR | Lymphoma | |
| SLC43A2 AND NOT-AXL | Kidney Chromophobe | 0.153846154 |
| NOT-MET AND IL13RA2 | Glioblastoma Multiforme | 0.153846154 |
| FAP AND NOT-GPC3 | Skin Cutaneous Melanoma | 0.153846154 |
| EPCAM AND NOT-KDR | Rectum Adenocarcinoma | 0.153846154 |
| NOT-ERBB2 AND BSG | Sarcoma | 0.151658768 |
| B4GALNT1 AND NOT-AXL | Liver Hepatocellular Carcinoma | 0.151371807 |
| CA9 AND NOT-GPC3 | Uveal Melanoma | 0.150943396 |
| NOT-KDR AND CA9 | Pancreatic Adenocarcinoma | 0.1504 |
| BSG AND NOT-CEACAM1 | Mesothelioma | 0.148148148 |
| CD70 AND MSLN AND NOT- | Mesothelioma | 0.148148148 |
| TMEM30B | | |
| FAP AND NOT-CEACAM5 | Thyroid Carcinoma | 0.144768439 |
| PRR7 AND NOT-ROR2 | Kidney Renal Papillary Cell Carcinoma | 0.144499179 |
| CA9 AND MUC16 | Uterine Corpus Endometrial Carcinoma | 0.144144144 |
| MUC1 AND NOT-GPC3 | Pancreatic Adenocarcinoma | 0.14242116 |
| NOT-ROR1 AND BSG | Cervical Squamous Cell Carcinoma and | 0.141962422 |
| | Endocervical Adenocarcinoma | |
| MUC1 AND FAP | Stomach Adenocarcinoma | 0.139884393 |
| MET AND NOT-MSLN | Liver Hepatocellular Carcinoma | 0.139714625 |
| NOT-CD274 AND CA9 | Uterine Carcinosarcoma | 0.139534884 |
| NOT-MUC1 AND B4GALNT1 | Testicular Germ Cell Tumors | 0.138173302 |
| CA9 AND NOT-AXL | Rectum Adenocarcinoma | 0.132231405 |
| NOT-LYVE1 AND ERBB2 | Breast Invasive Carcinoma | 0.13215859 |
| ROR2 AND MSLN | Mesothelioma | 0.131147541 |
| MUC1 AND L1CAM | Ovarian Serous Cystadenocarcinoma | 0.131060606 |
| ERBB2 AND NOT-IL13RA2 | Kidney Renal Papillary Cell Carcinoma | 0.128450704 |
| CD70 AND CD80 AND NOT- | Lymphoid Neoplasm Diffuse Large B-cell | 0.126760563 |
| GPC3 | Lymphoma | |
| B4GALNT1 AND NOT-ERBB2 | Glioblastoma Multiforme | 0.126368997 |
| KDR AND NOT-CEACAM5 | Thyroid Carcinoma | 0.126347709 |
| NOT-ROR1 AND MSLN | Rectum Adenocarcinoma | 0.125603865 |
| CA9 AND NOT-MSLN | Adrenocortical Carcinoma | 0.125490196 |
| NOT-PROM1 AND CD70 | Sarcoma | 0.123376623 |
| CA9 AND NOT-IL13RA2 | Breast Invasive Carcinoma | 0.120446097 |
| ERBB2 AND NOT-GPC3 | Breast Invasive Carcinoma | 0.119733925 |
| EPCAM AND NOT-AXL | Rectum Adenocarcinoma | 0.118483412 |
| NOT-MUC1 AND BSG | Skin Cutaneous Melanoma | 0.118343195 |
| NOT-CD274 AND FOLR1 | Ovarian Serous Cystadenocarcinoma | 0.116182573 |
| NOT-VIPR1 AND AXL | Sarcoma | 0.115555556 |
| ERBB2 AND NOT-AXL | Colon Adenocarcinoma | 0.115127175 |
| SCARB1 AND BSG | Adrenocortical Carcinoma | 0.114285714 |
| NOT-TMEM30B AND BSG | Mesothelioma | 0.114285714 |
| NKAIN1 AND NOT-EGFR | Uterine Carcinosarcoma | 0.113513514 |
| MUC1 AND B4GALNT1 | Stomach Adenocarcinoma | 0.111848341 |
| NOT-FOLH1 AND MSLN | Rectum Adenocarcinoma | 0.11 |
| NOT-EPCAM AND B4GALNT1 | Mesothelioma | 0.108108108 |
| NOT-KDR AND CA9 | Breast Invasive Carcinoma | 0.104615385 |
| TNFRSF9 AND NOT-EPHA2 | Lymphoid Neoplasm Diffuse Large B-cell | 0.104018913 |
| | Lymphoma | |
| NOT-SCN4B AND B4GALNT1 | Cholangiocarcinoma | 0.102564103 |
| TNFRSF9 AND NOT-PROM1 | Lung Squamous Cell Carcinoma | 0.102022552 |
| EPCAM AND NOT-GPC3 | Pancreatic Adenocarcinoma | 0.101172116 |
| CELSR3 AND NOT-GPC3 | Rectum Adenocarcinoma | 0.100694444 |
| CD274 AND NOT-AXL | Lung Squamous Cell Carcinoma | 0.099009901 |
| NOT-PROM1 AND BSG | Thyroid Carcinoma | 0.097457627 |
| TNFRSF9 AND NOT-GPC3 | Lymphoid Neoplasm Diffuse Large B-cell | 0.095671982 |
| | Lymphoma | |
| KREMEN2 AND BSG | Thymoma | 0.095238095 |
| CD8B AND BSG | Thymoma | 0.095238095 |
| MUC1 AND BSG | Ovarian Serous Cystadenocarcinoma | 0.093538794 |
| NOT-ROR2 AND MC1R | Cholangiocarcinoma | 0.093023256 |
| NOT-KDR AND BSG | Stomach Adenocarcinoma | 0.090313183 |
| BSG AND PTPRH | Pancreatic Adenocarcinoma | 0.08988764 |
| NOT-MUC1 AND BSG | Glioblastoma Multiforme | 0.088733217 |
| NOT-ROR1 AND EPCAM | Uterine Corpus Endometrial Carcinoma | 0.085621971 |
| BSG AND NOT-CEACAM1 | Sarcoma | 0.085561497 |
| NOT-EPHA2 AND AXL | Glioblastoma Multiforme | 0.08373079 |
| NOT-EGFR AND B4GALNT1 | Skin Cutaneous Melanoma | 0.08372093 |
| B4GALNT1 AND BSG | Stomach Adenocarcinoma | 0.08269096 |
| CELSR3 AND NOT-KDR | Pancreatic Adenocarcinoma | 0.082352941 |
| NOT-EPHA2 AND SLC43A2 | Kidney Chromophobe | 0.081632653 |
| BSG AND AXL | Sarcoma | 0.08 |
| CD70 AND NOT-GPC3 | Cervical Squamous Cell Carcinoma and | 0.078688525 |
| | Endocervical Adenocarcinoma | |
| NOT-PROM1 AND CA9 | Thymoma | 0.078580482 |
| CNIH2 AND NOT-ERBB2 | Testicular Germ Cell Tumors | 0.077825818 |
| MUC1 AND NOT-CD274 | Pancreatic Adenocarcinoma | 0.076233184 |
| NOT-GPC3 AND L1CAM | Uveal Melanoma | 0.07615894 |
| NOT-EGFR AND ROR2 | Uterine Carcinosarcoma | 0.075919336 |
| NOT-KDR AND CA9 | Uterine Carcinosarcoma | 0.075642965 |
| NOT-KDR AND CA9 | Thymoma | 0.072669826 |
| NOT-EPCAM AND AXL | Mesothelioma | 0.072289157 |
| NOT-FOLR1 AND B4GALNT1 | Mesothelioma | 0.071698113 |
| NOT-ABCB1 AND ERBB2 | Bladder Urothelial Carcinoma | 0.07079646 |
| TNFRSF9 AND NOT-AXL | Lung Squamous Cell Carcinoma | 0.07078711 |
| NOT-KDR AND PRR7 | Cholangiocarcinoma | 0.070175439 |
| NOT-MET AND CD70 | Testicular Germ Cell Tumors | 0.069651741 |
| NOT-EGFR AND CD70 | Testicular Germ Cell Tumors | 0.068627451 |
| NOT-KDR AND CA9 | Esophageal Carcinoma | 0.068522484 |
| NOT-ROR1 AND CD70 | Uveal Melanoma | 0.066420664 |
| NOT-MUC1 AND AXL | Glioblastoma Multiforme | 0.064989518 |
| BSG AND NOT-CEACAM1 | Thyroid Carcinoma | 0.062639821 |
| NOT-KDR AND ERBB2 | Bladder Urothelial Carcinoma | 0.062222222 |
| ERBB2 AND NOT-GPC3 | Bladder Urothelial Carcinoma | 0.062222222 |
| BSG AND NOT-GPC3 | Thyroid Carcinoma | 0.061946903 |
| CA9 AND NOT-CEACAM5 | Adrenocortical Carcinoma | 0.061705989 |
| FAP AND NOT-PROM1 | Lymphoid Neoplasm Diffuse Large B-cell | 0.061538462 |
| | Lymphoma | |
| AXL AND NOT-CEACAM5 | Kidney Renal Clear Cell Carcinoma | 0.060869565 |
| NOT-ERBB2 AND AXL | Kidney Renal Clear Cell Carcinoma | 0.059574468 |
| PRR7 AND NOT-ROR2 | Cholangiocarcinoma | 0.058823529 |
| CD274 AND NOT-FOLH1 | Thymoma | 0.058252427 |
| NOT-EGFR AND CD70 | Skin Cutaneous Melanoma | 0.057441253 |
| NOT-FOLR1 AND IL13RA2 | Adrenocortical Carcinoma | 0.056 |
| NOT-PROM1 AND IL13RA2 | Adrenocortical Carcinoma | 0.054474708 |
| NOT-MET AND CD70 | Acute Myeloid Leukemia | 0.052631579 |
| NOT-ERBB2 AND L1CAM | Testicular Germ Cell Tumors | 0.052561247 |
| NOT-FOLR1 AND CD70 | Esophageal Carcinoma | 0.052511416 |
| NOT-EPHA2 AND L1CAM | Testicular Germ Cell Tumors | 0.050123254 |
| NOT-ROR1 AND ERBB2 | Uterine Corpus Endometrial Carcinoma | 0.049382716 |
| ROR2 AND NOT-XK | Mesothelioma | 0.048484848 |
| NOT-KDR AND MC1R | Cholangiocarcinoma | 0.046511628 |
| NOT-TNFRSF9 AND EGFR | Brain Lower Grade Glioma | 0.046511628 |
| NOT-FOLR1 AND CD70 | Uveal Melanoma | 0.045801527 |
| NOT-AQP3 AND BSG | Thyroid Carcinoma | 0.045248869 |
| NOT-PROM1 AND CD274 | Thymoma | 0.045180723 |
| CA9 AND NOT-L1CAM | Cholangiocarcinoma | 0.044052863 |
| NOT-FOLR1 AND B4GALNT1 | Skin Cutaneous Melanoma | 0.043771044 |
| EGFR AND NOT-GPC3 | Brain Lower Grade Glioma | 0.043668122 |
| MET AND NOT-AXL | Kidney Chromophobe | 0.043668122 |
| NOT-EPHA2 AND MET | Kidney Chromophobe | 0.043668122 |
| NOT-KIAA1324 AND MET | Kidney Chromophobe | 0.043290043 |
| MUC1 AND NOT-ROR2 | Kidney Chromophobe | 0.042553191 |
| NOT-KDR AND BSG | Pancreatic Adenocarcinoma | 0.04238921 |
| FAP AND NOT-FOLR1 | Skin Cutaneous Melanoma | 0.04007286 |
| NOT-ROR1 AND BSG | Rectum Adenocarcinoma | 0.037004405 |
| CD80 AND NOT-FOLR1 | Sarcoma | 0.036939314 |
| CD70 AND NOT-GPC3 | Skin Cutaneous Melanoma | 0.036363636 |
| ULBP2 AND ERBB2 | Bladder Urothelial Carcinoma | 0.036036036 |
| B4GALNT1 AND BSG | Pancreatic Adenocarcinoma | 0.033018868 |
| BSG AND CD70 | Kidney Renal Clear Cell Carcinoma | 0.03271028 |
| BSG AND NOT-GPC3 | Kidney Renal Clear Cell Carcinoma | 0.031531532 |
| NOT-LGR6 AND MET | Kidney Chromophobe | 0.030674847 |
| CD80 AND NOT-PROM1 | Sarcoma | 0.030456853 |
| CA9 AND B4GALNT1 | Esophageal Carcinoma | 0.03030303 |
| FAP AND B4GALNT1 | Uterine Carcinosarcoma | 0.029461756 |
| NOT-MUC1 AND BSG | Thymoma | 0.028571429 |
| NOT-PROM1 AND CD274 | Lymphoid Neoplasm Diffuse Large B-cell | 0.028368794 |
| | Lymphoma | |
| BSG AND KISS1R | Kidney Renal Clear Cell Carcinoma | 0.028103044 |
| TNFRSF9 AND FAP | Esophageal Carcinoma | 0.027439024 |
| NOT-MUC1 AND L1CAM | Skin Cutaneous Melanoma | 0.027180068 |
| ROR2 AND NOT-AXL | Uterine Carcinosarcoma | 0.026101142 |
| TNFRSF9 AND EPHA2 | Esophageal Carcinoma | 0.025439128 |
| NOT-FOLH1 AND BSG | Rectum Adenocarcinoma | 0.02518224 |
| NOT-CD274 AND ERBB2 | Uterine Corpus Endometrial Carcinoma | 0.024691358 |
| NOT-GPR133 AND ERBB2 | Kidney Chromophobe | 0.024144869 |
| NOT-VIPR1 AND BSG | Kidney Renal Clear Cell Carcinoma | 0.023474178 |
| CA9 AND NOT-GPC3 | Cholangiocarcinoma | 0.019933555 |
| EPHA2 AND FAP | Esophageal Carcinoma | 0.018997707 |
| NOT-KDR AND B4GALNT1 | Esophageal Carcinoma | 0.018621974 |
| NOT-ABCA8 AND BSG | Cholangiocarcinoma | 0.018072289 |
| CD70 AND NOT-GPC3 | Acute Myeloid Leukemia | 0.016 |
| CD274 AND NOT-GPC3 | Lymphoid Neoplasm Diffuse Large B-cell | 0.014084507 |
| | Lymphoma | |
| BSG AND NOT-AXL | Liver Hepatocellular Carcinoma | 0.014084507 |
| FAP AND NOT-GPC3 | Cholangiocarcinoma | 0.014 |
| EPCAM AND NOT-IL13RA2 | Cholangiocarcinoma | 0.013300083 |
| EPCAM AND NOT-L1CAM | Cholangiocarcinoma | 0.013289037 |
| NOT-KDR AND CD70 | Thymoma | 0.01183432 |
| MUC1 AND NOT-IL13RA2 | Kidney Chromophobe | 0.01124498 |
| NOT-SCNN1B AND BSG | Cholangiocarcinoma | 0.01025641 |
| NOT-CD274 AND KREMEN2 | Cholangiocarcinoma | 0.009532888 |
| FAP AND NOT-CD274 | Cholangiocarcinoma | 0.009380863 |
| PAQR9 AND BSG | Liver Hepatocellular Carcinoma | 0.007874016 |
| NOT-MUC1 AND BSG | Liver Hepatocellular Carcinoma | 0.007751938 |
| B4GALNT1 AND NOT-AXL | Uterine Carcinosarcoma | 0.007616146 |
| CD274 AND NOT-ROR2 | Kidney Chromophobe | 0.007597341 |
| B4GALNT1 AND NOT-IL13RA2 | Kidney Chromophobe | 0.007575758 |
| SLC30A10 AND BSG | Liver Hepatocellular Carcinoma | 0.007490637 |
| NOT-KDR AND B4GALNT1 | Cholangiocarcinoma | 0.006640106 |
| CD274 AND NOT-MSLN | Kidney Chromophobe | 0.006407323 |
| B4GALNT1 AND NOT-MSLN | Kidney Chromophobe | 0.005194805 |
| NOT-KDR AND BSG | Cholangiocarcinoma | 0.005138746 |
| KDR AND B4GALNT1 | Kidney Renal Clear Cell Carcinoma | 0.004739336 |
| FOLH1 AND B4GALNT1 | Kidney Renal Clear Cell Carcinoma | 0.004474273 |
| CELSR3 AND B4GALNT1 | Esophageal Carcinoma | 0.004273504 |
| NOT-ROR2 AND B4GALNT1 | Cholangiocarcinoma | 0.004154549 |
| NOT-ROR2 AND BSG | Cholangiocarcinoma | 0.004140787 |

The foregoing analysis, based on available gene expression datasets, predicts that using Boolean antigen combinations can significantly improve the selectivity of tumor recognition and avoidance of normal tissue cross-reactivity. Thus, using Boolean multi-antigen detecting engineered T cells has the potential to have a major impact on cancer recognition and the development of next generation cellular therapies.

It was found that adding new antigens to current clinically actionable CAR targets via AND or AND-NOT Boolean recognition is predicted to significantly increase cancer versus normal tissue discrimination. Moreover, many novel antigen pairs that show even stronger and near ideal discrimination were identified. All cancers show at least several (>25) antigen pairs above a clustering score cutoff of 0.85, with many having thousands of strong pairs, suggesting a potential therapeutic avenue for all tumor types when using a pair of antigens. Furthermore, with the addition of triples, every cancer type examined here has a promising clustering-based score and an F₁ score above 0.5 (out of ideal 1.0). Thus, there are likely to be many options of multi-antigen signatures that could be used to recognize any one type of tumor.

2-3 antigen combinatorial circuits may be sufficient to achieve strong cancer versus normal tissue discrimination. Notably, when we examine the precision and recall of detection, for top performing gates it was observed that as the number of antigens used for detection is increased from two to three, mean precision approaches perfection while the recall declines (FIG. 5D). This suggests that further improvement in therapeutic cell discriminatory potential will require more narrow sub-classifications of tumor type; either by pathologic or molecular subtypes (e.g. triple negative vs HER2 positive vs hormone receptor positive breast cancer) of cancers defined in TCGA. This number of antigens also matches well with current synthetic biology tools, as integrating 2-3 receptor circuits is possible with current gene transfer methods (e.g. lentiviral transduction), while four or more receptor circuits may potentially need significant improvements in vector payload capacity.

Finally, optimal discrimination also involves setting antigen detection thresholds-exactly where the cutoff lines of high and low expression is important for discrimination (FIG. 1A). In cases where large distances between tumor and normal samples were observed, separation is extremely robust and consequently, shifting threshold cutoffs makes little difference in F₁. In the other cases, however, F₁ scores are highly sensitive to shifts in cutoffs. This is why the potential antigen combinations were first ranked by clustering scores, and then use a classifier to evaluate performance. Focusing primarily on maximizing separation distance ensures that more of our top pairs are robust to thresholding.

### REFERENCES

Binder, J. X., Pletscher-Frankild, S., Tsafou, K., Stolte, C., O'Donoghue, S. I., Schneider, R., and Jensen, L. J. (2014). COMPARTMENTS: unification and visualization of protein subcellular localization evidence. Database J. Biol. Databases Curation 2014, bau012.
de Cristofaro, T., Di Palma, T., Soriano, A. A., Monticelli, A., Affinito, O., Cocozza, S., and Zannini, M. (2016). Candidate genes and pathways downstream of PAX8 involved in ovarian high-grade serous carcinoma. Oncotarget 7, 41929-41947.
Fedorov, V. D., Themeli, M., and Sadelain, M. (2013). PD-1- and CTLA-4-based inhibitory chimeric antigen receptors (iCARs) divert off-target immunotherapy responses. Sci. Transl. Med. 5, 215ra172.
Ferguson, H. J. M., Wragg, J. W., Ward, S., Heath, V. L., Ismail, T., and Bicknell, R. (2016). Glutamate dependent NMDA receptor 2D is a novel angiogenic tumour endothelial marker in colorectal cancer. Oncotarget 7, 20440-20454.
Grada, Z., Hegde, M., Byrd, T., Shaffer, D. R., Ghazi, A., Brawley, V. S., Corder, A., Schönfeld, K., Koch, J., Dotti, G., et al. (2013). TanCAR: A Novel Bispecific Chimeric Antigen Receptor for Cancer Immunotherapy. Mol. Ther. Nucleic Acids 2, e105.
GTEx Consortium (2017). Genetic effects on gene expression across human tissues. Nature 550, 204-213.
Hegde, M., Corder, A., Chow, K. K. H., Mukherjee, M., Ashoori, A., Kew, Y., Zhang, Y. J., Baskin, D. S., Merchant, F. A., Brawley, V. S., et al. (2013). Combinational targeting offsets antigen escape and enhances effector functions of adoptively transferred T cells in glioblastoma. Mol. Ther. J. Am. Soc. Gene Ther. 21, 2087-2101.
Hie, B., Cho, H., DeMeo, B., Bryson, B., and Berger, B. (2019). Geometric Sketching Compactly Summarizes the Single-Cell Transcriptomic Landscape. Cell Syst. 8, 483-493.e7.
Hintzen, R. Q., Lens, S. M., Koopman, G., Pals, S. T., Spits, H., and van Lier, R. A. (1994). CD70 represents the human ligand for CD27. Int. Immunol. 6, 477-480.
Jilaveanu, L. B., Sznol, J., Aziz, S. A., Duchen, D., Kluger, H. M., and Camp, R. L. (2012). CD70 expression patterns in renal cell carcinoma. Hum. Pathol. 43, 1394-1399.
Johnson, W. E., Li, C., and Rabinovic, A. (2007). Adjusting batch effects in microarray expression data using empirical Bayes methods. Biostat. Oxf. Engl. 8, 118-127.
Kloss, C. C., Condomines, M., Cartellieri, M., Bachmann, M., and Sadelain, M. (2013). Combinatorial antigen recognition with balanced signaling promotes selective tumor eradication by engineered T cells. Nat. Biotechnol. 31, 71-75.
Lamers, C. H., Sleijfer, S., van Steenbergen, S., van Elzakker, P., van Krimpen, B., Groot, C., Vulto, A., den Bakker, M., Oosterwijk, E., Debets, R., et al. (2013). Treatment of metastatic renal cell carcinoma with CAIX CAR-engineered T cells: clinical evaluation and management of on-target toxicity. Mol. Ther. J. Am. Soc. Gene Ther. 21, 904-912.
Maude, S. L., Laetsch, T. W., Buechner, J., Rives, S., Boyer, M., Bittencourt, H., Bader, P., Verneris, M. R., Stefanski, H. E., Myers, G. D., et al. (2018). Tisagenlecleucel in Children and Young Adults with B-Cell Lymphoblastic Leukemia. N. Engl. J. Med. 378, 439-448.
Mbalaviele, G., Nishimura, R., Myoi, A., Niewolna, M., Reddy, S. V., Chen, D., Feng, J., Roodman, D., Mundy, G. R., and Yoneda, T. (1998). Cadherin-6 mediates the heterotypic interactions between the hemopoietic osteoclast cell lineage and stromal cells in a murine model of osteoclast differentiation. J. Cell Biol. 141, 1467-1476.
Morgan, R. A., Yang, J. C., Kitano, M., Dudley, M. E., Laurencot, C. M., and Rosenberg, S. A. (2010). Case Report of a Serious Adverse Event Following the Administration of T Cells Transduced With a Chimeric Antigen Receptor Recognizing ERBB2. Mol. Ther. 18, 843-851.
Morsut, L., Roybal, K. T., Xiong, X., Gordley, R. M., Coyle, S. M., Thomson, M., and Lim, W. A. (2016). Engineering Customized Cell Sensing and Response Behaviors Using Synthetic Notch Receptors. Cell 164, 780-791.
Neelapu, S. S., Locke, F. L., Bartlett, N. L., Lekakis, L. J., Miklos, D. B., Jacobson, C. A., Braunschweig, I., Oluwole, O. O., Siddiqi, T., Lin, Y., et al. (2017). Axicabtagene Ciloleucel CAR T-Cell Therapy in Refractory Large B-Cell Lymphoma. N. Engl. J. Med. 377, 2531-2544.
Parkhurst, M. R., Yang, J. C., Langan, R. C., Dudley, M. E., Nathan, D.-A. N., Feldman, S. A., Davis, J. L., Morgan, R. A., Merino, M. J., Sherry, R. M., et al. (2011). T Cells Targeting Carcinoembryonic Antigen Can Mediate Regression of Metastatic Colorectal Cancer but Induce Severe Transient Colitis. Mol. Ther. 19, 620-626.
Paul, R., Ewing, C. M., Robinson, J. C., Marshall, F. F., Johnson, K. R., Wheelock, M. J., and Isaacs, W. B. (1997). Cadherin-6, a cell adhesion molecule specifically expressed in the proximal renal tubule and renal cell carcinoma. Cancer Res. 57, 2741-2748.
Qu, X., Liu, J., Zhong, X., Li, X., and Zhang, Q. (2016). Role of AXL expression in non-small cell lung cancer. Oncol. Lett. 12, 5085-5091.
Rosenberg, S. A., and Restifo, N. P. (2015). Adoptive cell transfer as personalized immunotherapy for human cancer. Science 348, 62-68.
Roybal, K. T., Williams, J. Z., Morsut, L., Rupp, L. J., Kolinko, I., Choe, J. H., Walker, W. J., McNally, K. A., and Lim, W. A. (2016a). Engineering T Cells with Customized Therapeutic Response Programs Using Synthetic Notch Receptors. Cell 167, 419-432.e16.
Roybal, K. T., Rupp, L. J., Morsut, L., Walker, W. J., McNally, K. A., Park, J. S., and Lim, W. A. (2016b). Precision Tumor Recognition by T Cells With Combinatorial Antigen-Sensing Circuits. Cell 164, 770-779.
Srivastava, S., Salter, A. I., Liggitt, D., Yechan-Gunja, S., Sarvothama, M., Cooper, K., Smythe, K. S., Dudakov, J. A., Pierce, R. H., Rader, C., et al. (2019). Logic-Gated ROR1 Chimeric Antigen Receptor Expression Rescues T Cell-Mediated Toxicity to Normal Tissues and Enables Selective Tumor Targeting. Cancer Cell 35, 489-503.e8.
Sumia, I., Pierani, A., and Causeret, F. (2019). Kremenl-induced cell death is regulated by homo- and heterodimerization. Cell Death Discov. 5, 91.
Tesselaar, K., Xiao, Y., Arens, R., van Schijndel, G. M. W., Schuurhuis, D. H., Mebius, R. E., Borst, J., and van Lier, R. A. W. (2003). Expression of the murine CD27 ligand CD70 in vitro and in vivo. J. Immunol. Baltim. Md 1950 170, 33-40.
Thistlethwaite, F. C., Gilham, D. E., Guest, R. D., Rothwell, D. G., Pillai, M., Burt, D. J., Byatte, A. J., Kirillova, N., Valle, J. W., Sharma, S. K., et al. (2017). The clinical efficacy of first-generation carcinoembryonic antigen (CEACAM5)-specific CAR T cells is limited by poor persistence and transient pre-conditioning-dependent respiratory toxicity. Cancer Immunol. Immunother. CII 66, 1425-1436.
Wang, Q. J., Yu, Z., Hanada, K.-I., Patel, K., Kleiner, D., Restifo, N. P., and Yang, J. C. (2017). Preclinical Evaluation of Chimeric Antigen Receptors Targeting CD70-Expressing Cancers. Clin. Cancer Res. Off. J. Am. Assoc. Cancer Res. 23, 2267-2276.
Wilkie, S., van Schalkwyk, M. C. I., Hobbs, S., Davies, D. M., van der Stegen, S. J. C., Pereira, A. C. P., Burbridge, S. E., Box, C., Eccles, S. A., and Maher, J. (2012). Dual targeting of ErbB2 and MUC1 in breast cancer using chimeric antigen receptors engineered to provide complementary signaling. J. Clin. Immunol. 32, 1059-1070.
Xie, Z., Dang, Y., Wu, H., He, R., Ma, J., Peng, Z., Rong, M., Li, Z., Yang, J., Jiang, Y., et al. (2020). Effect of CELSR3 on the Cell Cycle and Apoptosis of Hepatocellular Carcinoma Cells. J. Cancer 11, 2830-2844.
Yu, H., Liu, R., Ma, B., Li, X., Yen, H.-Y., Zhou, Y., Krasnoperov, V., Xia, Z., Zhang, X., Bove, A. M., et al. (2015). Axl receptor tyrosine kinase is a potential therapeutic target in renal cell carcinoma. Br. J. Cancer 113, 616-625.
Zhu, C., Wei, Y., and Wei, X. (2019). AXL receptor tyrosine kinase as a promising anti-cancer approach: functions, molecular mechanisms and clinical applications. Mol. Cancer 18, 153.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An in vitro or ex vivo genetically modified cytotoxic immune cell, wherein the cytotoxic immune cell is genetically modified to produce at least two different polypeptides and wherein the polypeptides bind to a selected antigen doublet or triplet of Table 1.

2. The immune cell of claim 1, wherein the different polypeptides are independently selected from the group consisting of a binding-triggered transcriptional switch (BTTS), a chimeric antigen receptor (CAR), a T cell receptor (TCR), and an inhibitory CAR, depending on the Boolean operators associated with the selected combination of cell surface antigens.

3. The cell of claim 1, wherein the immune cell is genetically modified to produce two different polypeptides comprising a first polypeptide and a second polypeptide, wherein the first polypeptide specifically binds to a first antigen of a selected antigen doublet from Table 1 and the second polypeptide specifically binds to a second antigen of the selected antigen doublet.

4. The cell of claim 3, wherein the immune cell is only activated if the first and second polypeptides are both bound to an antigen.

5. The cell of claim 3, wherein the immune cell is not activated if the first and second peptides are both bound to an antigen.

6. The cell of claim 1, wherein the immune cell is genetically modified to produce three different polypeptides comprising a first polypeptide, a second polypeptide and a third polypeptide, wherein the first polypeptide specifically binds to a first antigen of a selected antigen doublet from Table 1, the second polypeptide specifically binds to a second antigen of the selected antigen doublet and the third polypeptide specifically binds to a third antigen of the selected antigen doublet.

7. The cell of claim 6, wherein the immune cell is only activated if the first, second and third polypeptides are bound to an antigen.

8. The cell of claim 6, wherein the immune cell is not activated if the first, second and third peptides are bound to an antigen

9. The cell of claim 1, wherein the polypeptides each comprise an extracellular binding domain independently selected from an antibody, peptide or ligand for a receptor.

10. The cell of claim 1, wherein the cell is a cytotoxic T cell.

11. A method of killing a target cancer cell in an individual, the method comprising:
administering to the individual an effective number of the genetically modified cytotoxic immune cell of claim 1, wherein said genetically modified cytotoxic immune cell kills the target cancer cell in the individual, and the type of cancer cell is associated with selected antigen doublet or triplet of Table 1.

12. A system for killing a target cancer cell, the system comprising:
a) a first antigen-triggered polypeptide that binds specifically to a first target antigen or a nucleic acid encoding the same; and
b) a second antigen-triggered polypeptide that binds specifically to a second target antigen or a nucleic acid encoding the same; and, optionally,
c) a third antigen-triggered polypeptide that binds specifically to a third target antigen or a nucleic acid encoding the same;
wherein the first, second and optional third antigen-triggered polypeptides bind to a selected antigen doublet or triplet of Table 1.

13. The system of claim 12, wherein the first, second and optional third polypeptides are independently selected from the group consisting of a binding-triggered transcriptional switch (BTTS), a chimeric antigen receptor (CAR), a T cell receptor (TCR), and an inhibitory CAR, depending on the Boolean operators associated with the selected combination of cell surface antigens.

14. A method of killing a target cancer cell in an individual, the method comprising:
a) introducing the system of any one of claim 12 into a cytotoxic T cell in vitro or ex vivo, generating a modified cytotoxic T cell; and
b) administering the modified cytotoxic T cell to the individual, wherein the target cancel cell is associated with selected antigen doublet or triplet of Table 1.

15. A polyspecific-immune-inducing polypeptide (PIIP) comprising:
a first antigen binding domain specific for a first antigen present on the surface of a target cancer cell, a second antigen binding domain specific for a second antigen present on the surface of the target cancer cell, and, optionally, a third antigen binding domain specific for a third antigen present on the surface of the target cancer cell
wherein the polyspecific-immune-inducing polypeptide binds a selected antigen doublet or triplet of Table 1.

16. The polyspecific-immune-inducing polypeptide of claim 15, wherein the PIIP is a polyspecific antibody.

17. The polyspecific-immune-inducing polypeptide of claim 15, wherein the PIIP is a polyspecific chimeric antigen receptor (CAR) or polyspecific T cell receptor (TCR).

18. An in vitro or ex vivo genetically modified cytotoxic immune cell, wherein the cytotoxic immune cell is genetically modified to produce a PIIP according to claim 15.

19. A method of killing a target cancer cell in an individual, the method comprising administering to the individual an effective amount of a PIIP according to claim 15.

20. The method according to claim 19, wherein the administering comprises administering to the individual an effective amount of cytotoxic immune cells genetically modified to produce the PIIP.
